(19) United States
(12) Patent Application Publication
(10) Pub. No.: US 2024/0011058 A1
(43) Pub. Date: Jan. 11, 2024

(54) CANNABIDIOLIC ACID (CBDA) SYNTHASE VARIANTS, AND MANUFACTURE AND USE THEREOF

(71) Applicant: Teewinot Life Sciences Corporation, New York, NY (US)

(72) Inventors: Jasleen BAINES, Dublin (IE); Oleg TYURIN, Dublin (IE); Malcolm J. KAVARANA, Fairfax, VA (US); Mingyang SUN, Dublin (IE)

(21) Appl. No.: 18/036,310

(22) PCT Filed: Nov. 11, 2021
(86) PCT No.: PCT/US2021/058964

§ 371 Date: May 10, 2023

Related U.S. Application Data
(60) Provisional application No. 63/113,274, filed on Nov. 13, 2020.

(51) Int. Cl. C12P 7/42; C12N 15/52; C12N 9/02; C12N 15/81

(52) U.S. Cl. CPC C12P 7/42; C12N 15/52; C12N 9/0004; C12N 15/815; C12Y 121/03008; C12R 2001/84

(57) ABSTRACT

Provided herein is a recombinant vector that comprises a promoter, a secretion tag gene of interest (GOI), and optionally a purification and/or a detection tag. The GOI comprises a polynucleotide sequence of SEQ ID NOs: 2-45 or a polynucleotide sequence with at least 90%, 95%, 98%, 99% or 99.9% identity with any one of SEQ ID NOs: 2-45. Also, disclosed are the sequences of polypeptides that result from expression of a GOI by a host expression system, and a method of producing cannabinoid compounds.

### CROSS-REFERENCE TO RELATED APPLICATIONS

The present application claims priority under 35 U.S.C. § 119(e) to U.S. Provisional Patent Application 63/113,274, filed Nov. 13, 2020, which is herein incorporated by reference in its entirety.

### BACKGROUND

Cannabinoids, the terpenophenolic compounds found in Cannabis sativa, have proven therapeutic potential. For example, cannabidiol (CBD) is a potent antioxidant and anti-inflammatory compound and may provide protection against acute and chronic neurodegeneration. It is found in high concentrations in hemp and acts as a high-affinity α2-adrenergic receptor agonist, a moderate-affinity 5-HT1A receptor antagonist, and a low-affinity CB1 receptor antagonist. CBD may also have anti-depressant activity. Cannabichromene (CBC) possesses anti-inflammatory, anti-fungal, and anti-viral properties. Thus, cannabinoids are regarded as promising therapeutic agents for the treatment of various diseases.

The varins are a class of cannabinoids that are structurally different from the classical cannabinoids (e.g., Tetrahydrocannabinol (THC), CBD, Cannabigerol (CBG), or CBC and their corresponding carboxylic acids). The varins have a 3-carbon propyl side chain instead of a pentyl (5-carbon) side chain attached to the aromatic ring. Many of the varins are found in very low amounts in the Cannabis plant. Tetrahydrocannabivarin (THCV) is one widely studied cannabinoid varin compounds. THCV can function as an antagonist of THC at CB1 receptors and thus attenuate the psychoactive effects of THC. THCV is being evaluated as a potential therapeutic for the treatment for type 2 diabetes, by increasing insulin sensitivity and improving glucose tolerance in patients with type-2 diabetes (Wargent et al., Nutr Diabetes., May; 3(5): e68 (2013)). THCV has also shown promise for the treatment of epilepsy and in reducing tremors associated with Parkinson's disease. Another minor cannabinoid is cannabidivarin (CBDV), an analog of cannabidiol. CBDV is presently being tested as a therapeutic for the treatment of adult epilepsy.

However, traditional chemical synthesis of many cannabinoids, particularly the low abundance varins is either suboptimal, non-existing, and/or does not permit the cost-effective manufacture of cannabinoids in high yields and at commercial scales. Thus, there is a need to develop a more effective system for industrial-scale manufacture of cannabinoid compounds. Bio-catalysis provides one cost-effective method for producing many cannabinoids found in the Cannabis plant. The process requires contacting a cannabinoid synthase enzyme with a substrate of the enzyme to produce cannabinoids and the low abundance varins at commercial scale and in pharmaceutical grade purity. While existing protocols, such as the one described in U.S. Pat. No. 10,336,978 is focused on using cannabinoid synthase enzymes, namely, CBDA synthase and THCA synthase found in the Cannabis plant, for the bio-catalytic manufacture of cannabinoids, the focus of this application is on mutants of CBDA synthase that have improved yields and enhanced kinetic rates compared to the plant (botanical) CBDA synthase enzyme.

### SUMMARY OF THE INVENTION

In one aspect, the present invention provides a recombinant vector that comprises a promoter, secretion tag, gene of interest (GOI), and optionally a purification and/or a detection tag, wherein the gene of interest comprises a polynucleotide sequence encoding a polypeptide with at least 90%, 95%, 98%, or 99.9% sequence identity to the sequence as set forth in any one of SEQ ID NOs: 72-115.

In another aspect, the present invention provides a polynucleotide comprising a polynucleotide sequence as set forth in any one of SEQ ID No. 2-45, or a polynucleotide sequence comprising at least 90%, 95%, 98%, 99%, or 99.9%, sequence identity to a sequence as set forth in any one of SEQ ID NOs: 2-45.

In some aspects, the present invention provides a polypeptide comprising the amino acid sequence of any one of SEQ ID NOs: 72-115 or a polypeptide comprising an amino acid sequence having at least 90%, 95%, 98%, or 99.9% sequence identity to a sequence as set forth in any one of SEQ ID NOs: 72-115.

In other aspects, the present invention provides a gene of interest operably linked to a promoter which is a polynucleotide as set forth in any one of SEQ ID NOs: 59-68. In one aspect, the present invention provides a promoter having a polynucleotide sequence comprising at least 90%, 95%, 98%, or 99% sequence identity to the sequence of SEQ ID NOs: 59-68.

In another aspect, the present invention provides a promoter that is a constitutive promoter or an inducible promoter. In one embodiment, the promoter is selected from a group consisting of an alcohol oxidase 1 promoter (pAOX1), an alcohol oxidase 2 promoter (pAOX2), a dihydroxyacetone kinase promoter (pDAK), an S-hydroxymethyl-glutathione hydrolase promoter (pFGH), an NADY-dependent formylglutathione dehydrogenase promoter (pFDH), a fructose 1,6-bisphosphate aldolase promoter (pFBA2), a peroxisomal membrane signal receptor PTS1 promoter (pPEX5), a peroxisomal protein Pex8p promoter (pPEX8), an alcohol dehydrogenase 2 promoter (pADH2) and a peroxin Pex14p promoter (pPEX14).

In some aspects, the present invention provides a recombinant vector comprising a secretion tag as set forth in any one of SEQ ID NOs: 46-55 and 130 or a secretion tag whose sequence is at least 90%, 95%, 98%, 99%, 99.9%, or 100% identical to SEQ ID NOs: 46-55 and 130.

In other aspects, the present invention provides a recombinant vector comprising a purification and/or a detection tag as set forth in any one of SEQ ID NOs: 56-58 and 129. In one embodiment, the purification tag is selected from the group consisting of a human influenza hemagglutinin (HA) tag, a His 6 tag and an HN tag.

In one aspect, the present invention provides a host expression system comprising a recombinant vector that comprises a promoter, secretion tag, gene of interest (GOI), and optionally a purification and/or a detection tag.

In another aspect, the present invention provides a host expression system comprising a microbial cell, a yeast cell, a plant cell, or an animal cell.

In some aspects, the present invention provides a host expression system comprising a yeast cell is selected from the group consisting of Pichia pastoris, Pichia angusta, Pichia guillermordii, Pichia methanolica, Pichia inositovera, Hansenula polymorpha, Candida boidinii, and Yarrowia lipolytica.

In other aspects, the present invention provides a method of producing a Cannabidiolic acid (CBDA) synthase or its variants. In another embodiment, the invention provides a method for producing a cannabinoid by contacting a cannabinoid precursor with an enzyme that is expressed by the recombinant vector of this disclosure.

### BRIEF DESCRIPTION OF THE DRAWINGS

FIG. 1 shows the expression cassette for a recombinant vector according to one embodiment of the invention.

FIG. 2 shows the high-pressure liquid chromatography (HPLC) plot of enzyme assay sample of CBDA synthase of SEQ ID NO: 71.

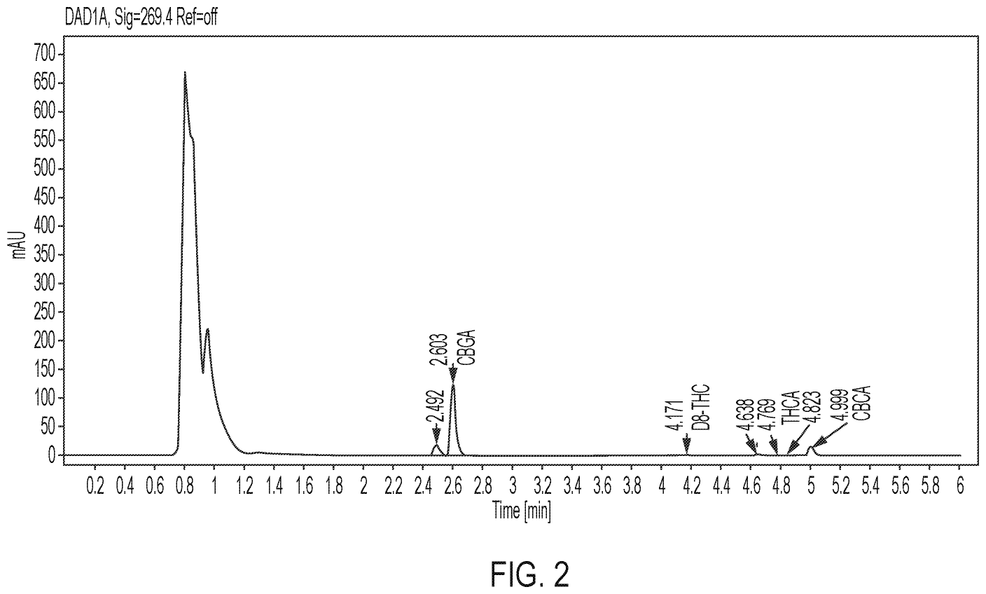

FIG. 3 shows the HPLC plot of enzyme assay sample of CBDA synthase of SEQ ID NO: 75.

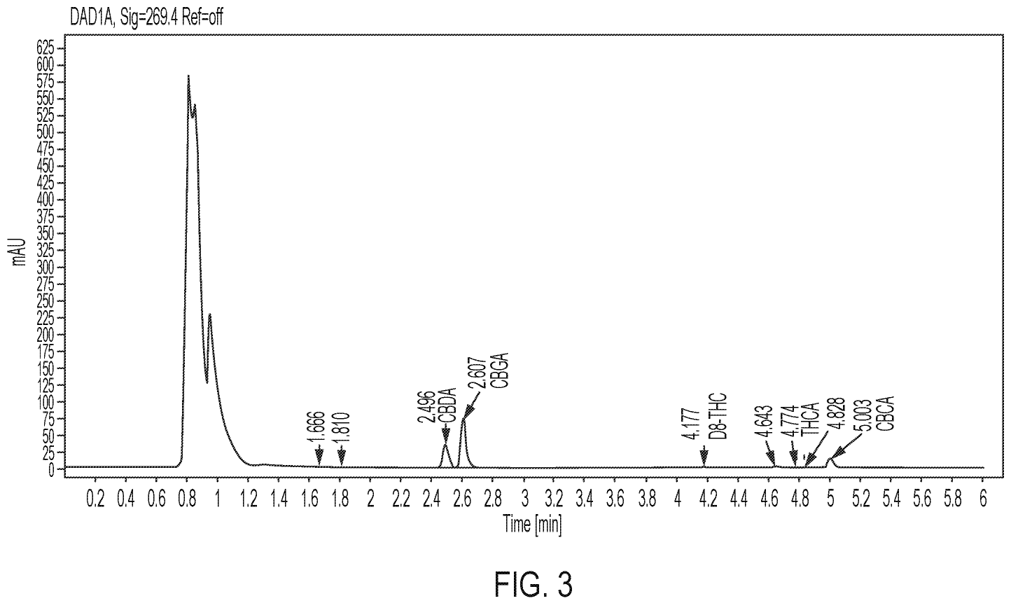

FIG. 4 shows the HPLC plot of enzyme assay sample of CBDA synthase of SEQ ID NO: 77.

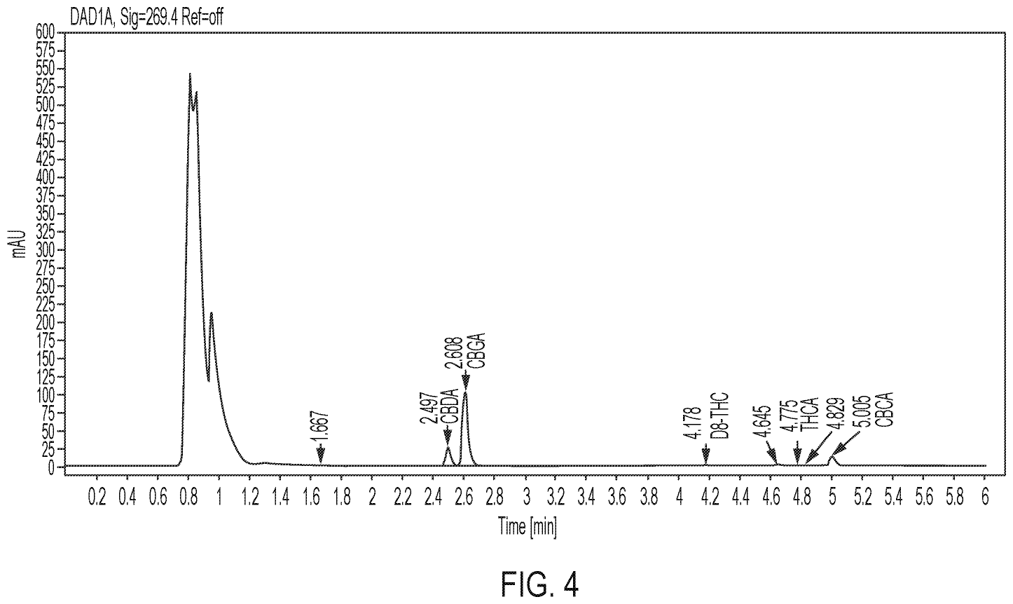

### DETAILED DESCRIPTION

It is to be understood that the invention is not limited to particular methodologies, protocols, cell lines, animal species or genera, compounds, polymers, and reagents described, as such may vary. It is also to be understood that the terminology used herein is for the purpose of describing particular embodiments only and is not intended to limit the scope of the present invention, which will be limited only by the appended claims.

Terms as set forth hereinafter are generally to be understood in their common sense unless indicated otherwise.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably +1%, and still more preferably +0.1% from the specified value, as such variations are appropriate to perform the disclosed methods.

The term “comprising” or “comprises” is intended to mean that the compositions and methods include the recited elements, but do not exclude others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than a trace amount of other ingredients and substantial method steps recited. Embodiments defined by each of these transition terms are within the scope of this invention.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “precursor” refers to a compound that participates in a chemical reaction that produces another compound. In one embodiment, the cannabinoid precursor refers to a compound that participates in a reaction to produce another compound.

For example, CBGA is a precursor to THCA, CBDA, and CBCA. In another example, CBGVA is a precursor to THCVA, CBDVA, and CBCVA.

The terms “nucleic acid” and “polynucleotide” as used herein are interchangeable. Nucleic acids and polynucleotides are polymers of nucleotides.

The nucleic acids of the present disclosure may include naturally occurring bases including adenine, guanine, cytosine, thymidine, and uracil. The sequences of nucleic acids may also contain modified bases. Examples of such modified bases include aza- and deaza-adenine, guanine, cytosine, thymidine and uracil; and xanthine and hypoxanthine. In one embodiment, the polynucleotide or nucleic acid molecule is a complementary DNA (cDNA).

A polypeptide, in one embodiment, may include various structural forms of the primary protein. For example, a polypeptide of the disclosure may be in the form of acidic or basic salts or in neutral form. In addition, individual amino acid residues may be modified by oxidation or reduction. The proteins and polypeptides of the present disclosure may also include truncations, analogs and homologs of the proteins and polypeptides, as described herein, having substantially the same function as the proteins or polypeptides of the present disclosure, such as having CBDA synthase activity.

The term “sequence identity” or “identity” refers to the maximum correspondence relation of the amino acid or nucleotide residues in the two sequences that are aligned for comparison. The length for identity comparison may extend to the full length of a nucleotide or polypeptide sequence, or a fragment of a nucleotide or polypeptide sequence that is at least about nine residues, at least about 20 to 24 residues, at least about 28 to 32 residues, and preferably at least about 36 or greater than 36 residues. Several different algorithms known in the art that can be used to measure sequence identity. In one embodiment, the sequence identity is in a range from about 50% to about 99.9%, about 55% to about 99.9%, about 60% to about 99.9%, about 65% to about 99.9%, about 70% to about 99.9%, about 75% to about 99.9%, about 80% to about 99.9%, about 85% to about 99.9%, about 90% to about 99.9%, and about 95% to about 99.9%.

In one embodiment, the sequence identity between two polynucleotides or two polypeptides is about 99.5%, about 98.5%, about 97.5%, about 96.5%, about 95.5%, about 94.5%, about 93.5%, about 92.5%, about 91.5%, about 90.5%, about 89.5%, about 88.5%, about 87.5%, about 86.5%, about 85.5%, about 84.5%, about 84.5%, about 83.5%, about 82.5%, about 81.5%, or about 80.5%.

In one embodiment, the sequence identity between two polynucleotides or two polypeptides is about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, or about 80%.

In one embodiment, the sequence identity between two polynucleotides or two polypeptides is about 99.9%, about 99.8%, about 99.7%, about 99.6%, about 99.5%, about 99.4%, about 99.3%, about 99.2%, about 99.1%, or about 99%.

The term “cannabinoid product” or “cannabinoid compound” is intended to mean any simple or complex substance or compound of natural, semi-synthetic, or synthetic origin, which can act on or modulate the activity of a cannabinoid receptor in a subject. In some embodiments, the cannabinoid product is an agonist of a cannabinoid receptor. In some embodiments, the cannabinoid product is an antagonist of a cannabinoid receptor. In yet another embodiment, the cannabinoid product is an inverse-agonist or an inverse-antagonist of a cannabinoid receptor. In one embodiment, the cannabinoid product comprises phytocannabinoids, endogenous cannabinoids (endocannabinoids), bio-synthetic cannabinoids, or synthetic cannabinoids. In one embodiment, the cannabinoid product comprises a pentyl side chain on the aromatic ring. Certain cannabinoids have a propyl side chain. In this application, this class of cannabinoids may be referred to as “varin.”

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

The present invention relates to mutants of CBDA synthase as well as recombinant vectors that comprises a polynucleotide sequences that code for CBDA synthase mutants. The CBDA synthase mutants of the invention have advantages over CBDA synthase found in the cannabis plant. For instance, the mutant CBDA synthases described here have better catalytic output compared to CBDA synthase in the cannabis plant. In one embodiment, the catalytic activity of the mutant CBDA synthases are at least 1-20 fold greater, at least 2-fold greater, at least 5-fold greater, at least 10-fold greater, at least 20-fold greater than the catalytic activity of the native CBDA synthase from the cannabis plant.

In some embodiments, for certain mutants, the catalytic activity is 1-20 fold greater than the catalytic activity of CBDA synthase enzyme from the cannabis plant. In one embodiment, the catalytic activity is 2-5 fold greater than the catalytic activity of CBDA synthase enzyme from the cannabis plant. In one embodiment, the catalytic activity of the mutants is 5-10 fold greater, or 16- or 20-fold greater than the catalytic activity of CBDA synthase from the cannabis plant.

In other embodiments, for certain mutants, the catalytic activity is about 1 to about 20 fold greater than the catalytic activity of a CBDA synthase having the sequence of SEQ ID NO: 71 or CBDA synthase of the cannabis plant (e.g., native CBDA synthase from a wild-type cannabis plant). In one embodiment, the catalytic activity of the mutant is about 2 to about 5 fold greater, about 5 to about 10 fold greater, about 16 fold greater, or about 20 fold greater than the catalytic activity of a CBDA synthase having the sequence of SEQ ID NO: 71 or CBDA synthase of the cannabis plant (e.g., native CBDA synthase from a wild-type cannabis plant).

Non-limiting cannabinoid products include tetrahydrocannabinol (THC), cannabidiol (CBD), olivetol, cannabinol (CBN), cannabigerol (CBG), cannabichromene (CBC), cannabicyclol (CBCL), nabilone, tetrahydrocannabinolic acid (THCA), cannabichromenic acid (CBCA), cannabicyclol carboxylic acid (CBCLA), cannabigerolic acid (CBGA), cannabidiolic acid (CBDA), cannabinolic acid (CBNA), tetrahydrocannabivarin (THCV), cannabivarin (CBV), cannabidivarin (CBDV), cannabigerovarin (CBGV), cannabichromevarin (CBCV), cannabicyclovarin (CBCLV), cannabicyclovarinic acid (CBCLVA), cannabigerovarinic acid (CBGVA), tetrahydrocannabivarinic acid (THCVA), cannabichrome varinic acid (CBCVA), cannabidivarinic acid (CBDVA), as well as the prodrugs and pharmaceutically acceptable salts of these cannabinoids. Exemplary prodrugs include alkyl ethers, haloalkyl ethers, alkyl esters, haloalkyl esters, and aromatic esters, for example CBD difluoromethyl ether or CBD methyl ether.

As used herein, the term “cannabinoid varin compound” refers to cannabinoid compounds comprising a propyl side chain attached to an aromatic ring. In one embodiment, the cannabinoid varin compound is psychoactive. In another embodiment, the cannabinoid varin compound is non-psychoactive. Non-limiting examples of cannabinoid varin compounds include tetrahydrocannabivarin (THCV), cannabivarin (CBV), cannabidivarin (CBDV), cannabigerovarin (CBGV), cannabichromevarin (CBCV), cannabicyclovarin (CBCLV), cannabicyclovarinic acid (CBCLVA), cannabigerovarinic acid (CBGVA), tetrahydrocannabivarinic acid (THCVA), cannabichromevarinic acid (CBCVA), and cannabidivarinic acid (CBDVA), as well as natural or synthetic molecules that have a basic cannabinoid varin structure and are modified synthetically to provide a cannabinoid analog.

The polynucleotide of interest can be cloned into a vector of interest to produce a construct. In an embodiment, the vector is an expression vector. Examples of expression vectors include but are not limited to cosmids, plasmids, or modified viruses (e.g. replication defective retroviruses, adenoviruses, and adeno-associated viruses), so long as the vector is compatible with the host cell used. The expression vectors are suitable for transformation of a host cell, which means that the expression vectors contain a polynucleotide of the application and regulatory sequences selected on the basis of the host cells to be used for expression. “Operably linked” is intended to mean that the nucleic acid is linked to regulatory sequences in a manner which allows expression of the nucleic acid. In some embodiments, the isolated and/or purified nucleic acid molecules, polynucleotides or vectors, constructs or in vitro expression systems comprising these isolated and/or purified nucleic acid molecules, may be used to create transgenic or recombinant organisms or recombinant cells (e.g. optionally cells of recombinant organisms) that produce polypeptides with cannabinoid synthase activity and/or modulated levels of polypeptides with cannabinoid synthase activity.

In some embodiments, a polynucleotide comprising a sequence encoding a polypeptide described herein, or a vector comprising the polynucleotide, can be codon-optimized e.g., for use in a particular host cell, for example, a bacterial or plant cell.

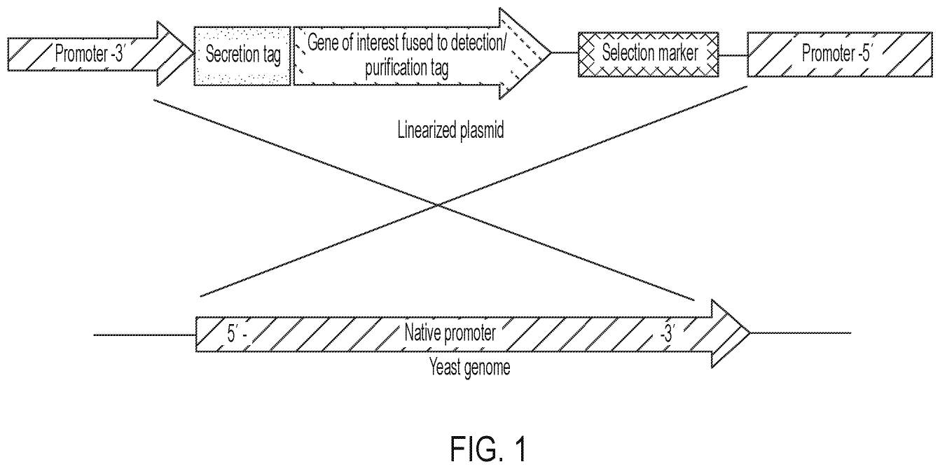

FIG. 1 schematically shows an expression cassette used for the manufacture of a CBDA synthase enzyme. In one embodiment, the expression cassette comprises a gene of interest (GOI) comprising a sequence as set forth in any one of SEQ ID NOs: 2-45 are operably linked to a secretion tag according SEQ ID NOs: 46-55 and 130, which in turn is operably linked to a promoter sequence as set forth in any one of SEQ ID Nos 59-68. The expression cassette is inserted into the yeast genome and is used to express a cannabinoid synthase enzyme. In most cases, a purification/detection tag as set forth in any one of SEQ ID Nos 56-58 and 129 is operably linked downstream of the gene of interest to enable purification and/or detection of the protein expressed by the gene of interest.

In one embodiment, the disclosure provides a recombinant vector comprising a polynucleotide sequence for the gene of interest (GOI) as set forth in any one of SEQ ID NOs: 2-45, or a recombinant vector comprising a polynucleotide sequence with at least 90%, 95%, 98%, or 99.9% identity with any one of SEQ ID NOs: 2-45.

In one embodiment, a polynucleotide comprising the polynucleotide sequence as set forth in any one of SEQ ID NOs: 2-45 encodes a polypeptide of SEQ ID NOs: 72-115. In one embodiment, the polynucleotide sequence encodes a polypeptide with at least 90%, 95%, 98%, or 99.9% identity with any one of SEQ ID NOs: 72-115.

In one embodiment, the polynucleotide sequence is operably linked to a regulatory sequence for the transcription and translation of the gene of interest (GOI). Suitable regulatory sequences may be derived from a variety of sources, including bacterial, fungal, viral, mammalian, or insect genes (for example, see the regulatory sequences described in Goeddel, Gene Expression Technology: Methods in Enzymology 185, Academic Press, San Diego, CA (1990), incorporated herein by reference in its entirety). Selection of appropriate regulatory sequences is dependent on the host cell chosen, as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

In one embodiment, the regulatory sequence is a promoter. The promoter is functional in the host cell or host expression system. In another embodiment, the promoter is a constitutive promoter or an inducible promoter. In one embodiment, the promoter is selected from a group consisting of an alcohol oxidase 1 promoter (pAOX1), an alcohol oxidase 2 promoter (pAOX2), a dihydroxyacetone kinase promoter (pDAK), an S-hydroxymethyl-glutathione hydrolase promoter (pFGH), a NAD⁺-dependent formylglutathione dehydrogenase promoter (pFDH), a peroxin Pex14p promoter (pPEX14p), a fructose 1,6-bisphosphate aldolase promoter (pFBA), a peroxisomal membrane signal receptor PTS1 promoter (pPEX5), an alcohol dehydrogenase 2 promoter (pADH2), and a peroxisomal protein Pex8p promoter (pPEX8).

In one embodiment, the promoter is a polynucleotide of SEQ ID NOs: 59-68 or a polynucleotide that has at least 90%, 95%, 98%, or 99% identity with any one of SEQ ID NOs: 59-68.

For some embodiments, the promoter is a polynucleotide that has at least 99% identity to a polynucleotide as set forth in any one of SEQ ID NOs: 59-68.

According to one embodiment, the promoter is a polynucleotide that has at least 98% identity to a polynucleotide as set forth in any one of SEQ ID NOs: 59-68.

In one embodiment, the promoter is a polynucleotide that has at least 97% identity to a polynucleotide as set forth in any one of SEQ ID NOs: 59-68.

In one embodiment, the promoter is a polynucleotide that has at least 96% identity to a polynucleotide as set forth in any one of SEQ ID NOs: 59-68.

In one embodiment, the promoter is a polynucleotide that has at least 95% identity to a polynucleotide as set forth in any one of SEQ ID NOs: 59-68.

In one embodiment, the promoter is a polynucleotide that has at least 90%, 91%, 92%, 93%, 94%, or 95% identity to a polynucleotide as set forth in any one of SEQ ID NOs: 59-68.

In one embodiment, the promoter is a polynucleotide sequence selected from a group consisting of SEQ ID NOs: 59-68.

In one embodiment, the vector encodes a polynucleotide for a secretion peptide. In one embodiment, the secretion peptide is encoded by a polynucleotide sequence of SEQ ID NOs: 46-55 and 130 or having at least 90%, 95%, 98%, 99%, or 100% identity with a polynucleotide of one of SEQ ID NOs: 46-55 and 130.

In one embodiment, the secretion peptide is encoded by a polynucleotide sequence having at least 99% identity with any one of SEQ ID NOs: 46-55 and 130.

In one embodiment, the secretion peptide is encoded by a polynucleotide sequence having at least 98% identity with any one of SEQ ID NOs: 46-55 and 130.

In one embodiment, the secretion peptide is encoded by a polynucleotide sequence having at least 97% identity with any one of SEQ ID NOs: 46-55 and 130.

In one embodiment, the secretion peptide is encoded by a polynucleotide sequence having at least 96% identity with any one of SEQ ID NOs: 46-55 and 130.

In one embodiment, the secretion peptide is encoded by a polynucleotide sequence having at least 95% identity with any one of SEQ ID NOs: 46-55 and 130.

In one embodiment, the secretion peptide is encoded by a polynucleotide sequence having at least 90%, at least 91%, at least 92%, at least 93%, at least 94%, or at least 95% identity with any one of SEQ ID NOs: 46-55 and 130.

In one embodiment, the secretion tag is encoded by a polynucleotide sequence that represents a codon optimized version of SEQ ID NO: 46. In some embodiments, the sequence for the codon optimized polynucleotide sequence is as follows (SEQ ID NO: 130):

ATGAGATTCCCATCCATCTTCACTGCTGTTTTGTTCGCTGCTTCTTCCG
CTTTGGCTGCTCCAGTTAACACTACTACTGAGGACGAGACTGCTCAGAT
TCCAGCTGAAGCTGTTATTGGTTACTCCGACTTGGAAGGTGACTTCGAC
GTTGCTGTTTTGCCATTCTCCAACTCCACTAACAACGGTTTGTTGTTCA
TCAACACTACAATCGCTTCCATTGCTGCTAAAGAAGAGGGAGTTTCCTT
GGAGAAGAGAGAGGCTGAAGCT

In another embodiment, the secretion peptide comprises a polypeptide sequence as set forth in any one of SEQ ID NOs: 116-125 or a polypeptide sequence having at least 90%, 95%, 98%, 99%, or 100% identity with any one of SEQ ID NOs: 116-125.

In one embodiment, the secretion peptide comprises a polypeptide sequence having at least 99% identity with any one of SEQ ID NOs: 116-125.

In one embodiment, the secretion peptide comprises a polypeptide sequence having at least 98% identity with any one of SEQ ID NOs: 116-125.

In one embodiment, the secretion peptide comprises a polypeptide sequence having at least 97% identity with any one of SEQ ID NOs: 116-125.

In one embodiment, the secretion peptide comprises a polypeptide sequence having at least 96% identity with any one of SEQ ID NOs: 116-125.

In one embodiment, the secretion peptide comprises a polypeptide sequence having at least 95% identity with any one of SEQ ID NOs: 116-125.

In one embodiment, the secretion peptide comprises a polypeptide sequence having at least 90% identity, at least 91% identity, at least 92% identity, at least 93% identity, or at least 94% identity with any one of SEQ ID NOs: 116-125.

In one embodiment, the vector encodes a polynucleotide for a purification and/or a detection tag. In another embodiment, the purification and/or detection tag is selected from the group consisting of a human influenza hemagglutinin (HA) tag, a His 6 tag, and an HN tag. In one embodiment, the purification tag is encoded by a polynucleotide sequence as set forth in any one of SEQ ID NOs: 56-58 and 129. In another embodiment, the purification tag encodes a polypeptide as set forth in any one of SEQ ID NOs: 126-128.

In one embodiment, the recombinant vector is a plasmid. Exemplary plasmids include the pPIC 3.5 and pL, represented by SEQ ID NOs: 69 and 70, respectively.

In one embodiment, the disclosure relates to a polynucleotide for the GOI as set forth in any one of SEQ ID NOs: 2-45. In another aspect, the disclosure relates to a polynucleotide of the GOI comprising a sequence having at least 90%, 95%, 98%, 99%, or 99.9% identity with any one of SEQ ID NOs: 2-45.

In one embodiment, the polynucleotide comprises a sequence having at least 99.9% identity with any one of SEQ ID NOs: 2-45.

In one embodiment, the polynucleotide comprises a sequence having at least 98% identity with any one of SEQ ID NOs: 2-45.

In one embodiment, the polynucleotide comprises a sequence having at least 97% identity with any one of SEQ ID NOs: 2-45.

In one embodiment, the polynucleotide comprises a sequence having at least 96% identity with any one of SEQ ID NOs: 2-45.

In one embodiment, the polynucleotide comprises a sequence having at least 95% identity with any one of SEQ ID NOs: 2-45.

In one embodiment, the polynucleotide comprises a sequence having at least 90%, at least 91%, at least 92%, at least 93%, or at least 94% identity with any one of SEQ ID NOs: 2-45.

In another aspect, the polynucleotide for the gene of interest (GOI) has a sequence as set forth in any one of SEQ ID NOs: 2-45.

In one embodiment, a polynucleotide of SEQ ID NOs: 2-45 encodes a polypeptide of SEQ ID NOs: 72-115. In one embodiment, a polynucleotide sequence having 90%, 95%, 98%, or 99.9% identity to SEQ ID NOs: 2-45 encodes a polypeptide with a sequence having at least at least 90%, 95%, 98%, or 99.9% identity with any one of SEQ ID NOs: 72-115.

In one embodiment, the polypeptide has 99.9% identity to a polypeptide of SEQ ID NOs: 72-115.

In another embodiment, the polypeptide has 98% identity to a polypeptide of SEQ ID NOs: 72-115.

In another embodiment, the polypeptide has 97% identity to a polypeptide of SEQ ID NOs: 72-115.

In another embodiment, the polypeptide has 96% identity to a polypeptide of SEQ ID NOs: 72-115.

In another embodiment, the polypeptide has 95% identity to a polypeptide of SEQ ID NOs: 72-115.

In one embodiment, the polypeptide has 90% identity, 91% identity, 92% identity, 93% identity, or 94% identity to a polypeptide of SEQ ID NOs: 72-115.

In another aspect, the present invention provides a polypeptide comprising the amino acid sequence of any one of SEQ ID NOs: 72-115 or an amino acid sequence comprising at least 90% identity, 91% identity, 92% identity, 93% identity, or 94% identity to a polypeptide of SEQ ID NOs: 72-115.

In some embodiments, a polypeptide of the present invention comprises an alteration at one or more (e.g., several) amino acids in the polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123,124, 125, 126, 127,128, 129, 130, 131, 132, 133, 134,135, 136, 137, 138, 139, 140, 141, 142,143, 144, 145, 146,147, 148, 149, 150, 151, 152, 153,154, 155, 156, 157, 158, 159, 160, 161,162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more amino acids are altered. In one embodiment, the alteration is relative to the reference polypeptide of SEQ ID NO: 71, wherein the alteration comprises one or more substitutions, insertions, deletions, and/or additions in the polypeptide relative to the reference polypeptide of SEQ ID NO: 71.

In other embodiments, the invention includes an isolated, recombinant, substantially pure, or non-naturally occurring polypeptide having CBDA synthase activity, wherein the polypeptide comprises an amino acid sequence comprising at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any one of SEQ ID NOs: 72-115. In one embodiment, the polypeptide does not comprise the sequence of SEQ ID NO: 71. In another embodiment, the CBDA synthase activity is at least about 2 to about 30-fold greater than the activity of a naturally occurring cannabis synthase or a synthase having the sequence of SEQ ID NO: 71.

The recombinant vector of the disclosure may also contain a selectable “marker gene”, which facilitates the selection of host cells transformed or transfected with a recombinant cassette of the application. Examples of selectable marker genes are genes encoding for proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin, optionally IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance, such as neomycin, resistance transformant cells can be selected with G418. Cells that have incorporated the selectable marker gene will survive, while the other cells die. In another embodiment, the selectable marker is introduced to the host expression system on a separate vector from the recombinant vector of interest.

The recombinant vectors of the disclosure can be introduced into a host cell or a host expression system to produce a transformed host cell. Prokaryotic and/or eukaryotic cells can be transformed with nucleic acid by, for example, electroporation or calcium chloride-mediated transformation. For example, the vector can be introduced into mammalian cells via conventional techniques such as calcium phosphate or calcium chloride co-precipitation, DEAE-dextran mediated transfection, lipofectin, viral mediated methods, electroporation or microinjection. Suitable methods for transforming and transfecting cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic cells and prokaryotic cells. For example, the nucleic acids and proteins of the disclosure may be expressed in plant cells, yeast cells or mammalian cells. In one embodiment, plant cells are of the genus Cannabis, for example Cannabis saliva L, Cannabis indica Lam, and Cannabis ruderalis Janisch, especially Cannabis sativa. Microorganisms are preferably bacteria (e.g. Escherichia coli) or yeast (e.g. Saccharomyces cerevisiae, Pichia pastoris). Insect cells are preferably Spodoptera frugiperda cells. The eukaryotic cell, in one embodiment, is a yeast cell.

Therefore, in another aspect, the disclosure provides a host expression system comprising the recombinant vector as disclosed herein. In one embodiment, the host expression system comprises a microbial cell, a yeast cell, a plant cell, or an animal cell. In another embodiment, the host expression system comprises a yeast cell. In one embodiment, the yeast comprises one or more of Pichia pastoris, Pichia angusta, Pichia guillermordii, Pichia methanolica, Pichia inositovera, Hansenula polymorpha, Candida boidinii, and Yarrowia lipolytica.

Accordingly, also provided herein is a recombinant cell comprising the recombinant vector or the polynucleotide of this disclosure. In one embodiment, the recombinant cell results in production of the polypeptide of this disclosure.

In another embodiment, the present disclosure provides a method of producing a Cannabidiolic acid (CBDA) synthase or its variants, which comprises expressing the recombinant vector as disclosed above in the host expression system.

In one embodiment, a method of producing a Cannabidiolic acid (CBDA) synthase or its variants comprises expressing the recombinant vector with a polynucleotide sequence as set forth in any one of SEQ ID NOs: 2-45.

In an embodiment, the method of producing a Cannabidiolic acid (CBDA) synthase or its variants comprises expressing the recombinant vector with a polynucleotide sequence having at least 90%, 95%, 98%, or 99.9% identity with any one of SEQ ID NOs: 2-45.

In an embodiment, the method of producing a Cannabidiolic acid (CBDA) synthase or its variants comprises expressing the recombinant vector with a polynucleotide sequence with at least 99.9% identity with any one of SEQ ID NOs: 2-45.

In an embodiment, the method of producing a Cannabidiolic acid (CBDA) synthase or its variants comprises expressing the recombinant vector with a polynucleotide sequence with at least 98% identity with any one of SEQ ID NOs: 2-45.

In an embodiment, the method of producing a Cannabidiolic acid (CBDA) synthase or its variants comprises expressing the recombinant vector with a polynucleotide sequence with at least 95% identity with any one of SEQ ID NOs: 2-45.

In an embodiment, the method of producing a Cannabidiolic acid (CBDA) synthase or its variants comprises expressing the recombinant vector with a polynucleotide sequence with at least 90% identity with any one of SEQ ID NOs: 2-45.

In an embodiment, the method of producing a Cannabidiolic acid (CBDA) synthase or its variants comprises expressing the recombinant vector with a polynucleotide sequence with at least 85%, 86%, 87%, 88% or 89% identity with any one of SEQ ID NOs: 2-45.

According to an embodiment, the recombinant vector with a polynucleotide sequence as set forth in any one of SEQ ID NOs: 2-45 encodes a polypeptide of SEQ ID NOs: 72-115 or a polypeptide with at least 90%, 95%, 98%, or 99% identity with any one of SEQ ID NOs: 72-115.

In one embodiment, the recombinant vector with a polynucleotide sequence as set forth in any one of SEQ ID NOs: 2-45 encodes a polypeptide with at least 99.9% identity with any one of SEQ ID NOs: 72-115.

In one embodiment, the recombinant vector with a polynucleotide sequence as set forth in any one of SEQ ID NOs: 2-45 encodes a polypeptide with at least 98% identity with any one of SEQ ID NOs: 72-115.

In one embodiment, the recombinant vector with a polynucleotide sequence as set forth in any one of SEQ ID NOs: 2-45 encodes a polypeptide with at least 95% identity with any one of SEQ ID NOs: 72-115.

In one embodiment, the recombinant vector with a polynucleotide sequence as set forth in any one of SEQ ID NOs: 2-45 encodes a polypeptide with at least 90% identity with any one of SEQ ID NOs: 72-115.

In one embodiment, the recombinant vector with a polynucleotide sequence as set forth in any one of SEQ ID NOs: 2-45 encodes a polypeptide with at least 85%, 86%, 87%, 88% or 89% identity with any one of SEQ ID NOs: 72-115.

In one embodiment the recombinant vector comprises a promoter of SEQ ID No. 59 operably linked to a secretion tag of SEQ ID No. 46 which is operably linked to the gene of interest of SEQ ID No. 5 operably linked to Seq ID No. 56.

In one embodiment the recombinant vector comprises a promoter of SEQ ID No. 60 operably linked to a secretion tag of SEQ ID No. 46 which is operably linked to the gene of interest of SEQ ID No. 5 operably linked to Seq ID No. 56.

In one embodiment the recombinant vector comprises a promoter of SEQ ID No. 63 operably linked to a secretion tag of SEQ ID No. 46 which is operably linked to the gene of interest of SEQ ID No. 5 operably linked to Seq ID No. 56.

In one embodiment the recombinant vector comprises a promoter of SEQ ID No. 64 operably linked to a secretion tag of SEQ ID No. 46 which is operably linked to the gene of interest of SEQ ID No. 5 operably linked to Seq ID No. 56.

In one embodiment the recombinant vector comprises a promoter of SEQ ID No. 59 operably linked to a secretion tag of SEQ ID No. 46 which is operably linked to the gene of interest of SEQ ID No. 13 operably linked to Seq ID No. 56.

In one embodiment the recombinant vector comprises a promoter of SEQ ID No. 60 operably linked to a secretion tag of SEQ ID No. 46 which is operably linked to the gene of interest of SEQ ID No. 13 operably linked to Seq ID No. 56.

In one embodiment the recombinant vector comprises a promoter of SEQ ID No. 63 operably linked to a secretion tag of SEQ ID No. 46 which is operably linked to the gene of interest of SEQ ID No. 13 operably linked to Seq ID No. 56.

In one embodiment the recombinant vector comprises a promoter of SEQ ID No. 64 operably linked to a secretion tag of SEQ ID No. 46 which is operably linked to the gene of interest of SEQ ID No. 13 operably linked to Seq ID No. 56.

In one embodiment, a method for producing a cannabinoid compound comprises reacting a cannabinoid precursor with an enzyme that is expressed by a host expression system comprising a recombinant vector that has a promoter operably linked to a secretion tag that is operably linked to a GOI, wherein the cannabinoid precursor is a compound of Formula I:

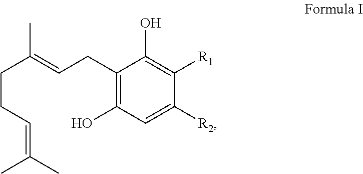

wherein R₁ is H or —COOH and R₂ is a linear or branched CH₃, C₂H₅, C₃H₇, C₄H₉, C₅H₁₀, C₆H₁₃, C₇H₁₅ or C₈H₁₇ group.

In one embodiment, the recombinant vector comprises a GOI having a polynucleotide sequence as set forth in any one of SEQ ID NOs: 2-45.

In an embodiment, the recombinant vector comprises a polynucleotide sequence with at least 90%, 95%, 98%, or 99.9% identity with any one of SEQ ID NOs: 2-45.

In one embodiment, the recombinant vector with a polynucleotide sequence as set forth in any one of SEQ ID NOs: 2-45 encodes a polypeptide as set forth in any one of SEQ ID NOs: 72-115.

In one embodiment, the recombinant vector with a polynucleotide sequence as set forth in any one of SEQ ID NOs: 2-45 encodes a polypeptide with at least 90%, 95%, 98%, or 99.9% identity with any one of SEQ ID NOs: 72-115.

In one embodiment, the cannabinoid compound produced by a method described in this application is isolated and/or purified using chemical purification protocols well known in the art. In one embodiment, R₂ is C₃H₇ or C₅H₁₀. In one embodiment, R₂ is C₃H₇ and R₁ is —COOH or and ester. In one embodiment, R₂ is C₅H₁₁ and R₁ is —COOH or and ester, for example a C₁-C₁₀ alkyl ester, an optionally substituted aryl or benzyl ester. In another embodiment, the cannabinoid precursor is CBGVA, CBGA, and ester of CBGA or CBGVA, or derivatives and analogs of CBGA and CBGVA.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Molecular Cloning

An array of promoters was employed to drive the expression of the native and mutant CBDA synthase enzymes. The suitable promoters include, but are not limited to: alcohol oxidase 1 promoter (pAOX1), alcohol oxidase 2 promoter (pAOX2), NAD⁺-dependent formylglutathione dehydrogenase promoter (pFDH), peroxin Pex14p promoter (pPEX14), S-hydroxymethyl-glutathione hydrolase promoter (pFGH), dihydroxyacetone kinase promoter (pDAK), fructose 1,6-bisphosphate aldolase promoter (pFBA2), peroxisomal protein Pex8p promoter (pPEX8), peroxisomal membrane signal receptor PTS1 promoter (pPEX5) and alcohol dehydrogenase 2 promoter (pADH2). In one embodiment, the pAOX1 promoter was cloned to the standard plasmid pPIC3.5 (Invitrogen) with the BglII and EcoRI restriction sites, yielding a derivative called plasmid pL. DH5alpha E. coli strain was used for cloning and plasmid amplification. Transformation was performed according to a standard heat-shock protocol as known in the art.

In order to get the enzyme to secrete into the culture medium, a secretion peptide was fused to the 5′ of the GOI (gene of interest) in frame. Suitable secretion tags include but are not limited to α-MF (alpha mating factor from S. cerevisiae yeast), HSP150 (Heat shock protein 150 from P. pastoris yeast), combination of pre-α-MF (19 amino acids from α-MF) and the pro-region of HSP150 from P. pastoris, combination of pre-α-MF and a double repeat of the pro-region of HSP150 from P. pastoris, SUC2 (invertase from S. cerevisiae), PHO1 (acid phosphatase from S. cerevisiae), PIR1 (55 amino acids from Protein with internal repeats 1, from P. pastoris), OST1 (dolichyl-diphosphooligosaccharide protein glycotransferase subunit OST1), OST1 fused to the pro-region of the α-MF secretion peptide (20-85 amino acids of the α-MF secretion peptide) and DDDK (first 18 amino acids from protein AOA68487.1:GenBank accession No. from P. pastoris).

In one embodiment, the secretion tag, and the gene for CBDA synthase or its mutants were synthesized as one construct. In another embodiment, a PCR based method was used to fuse the desired secretion tag to the codon optimized gene of CBDA synthase and mutants of this enzyme. The genes for the CBDA synthase and its mutants were chemically synthesized (Thermo Fisher Scientific, USA), and sequences of these constructs were verified with Sanger sequencing (Eurofins Genomics).

To process a secretion tagged construct (i.e., cleavage of the signal peptide from the nascent peptide chain), the cleavage recognition amino acid sequence (lysine-arginine) of Kex2 protease was introduced downstream of each secretion peptide. Thus, all secretion peptides were flanked with a lysine-arginine (KR) or lysine-lysine (KK) pair at the C-terminus, upstream of the respective GOI. To increase efficiency of cleavage by Kex2 protease, the amino acid pairs LE (leucine-glutamic acid) or ME (Methionine-glutamic acid) were cloned upstream of KR or KK flanks. Downstream of KR or KK cleavage site an EAEA repeat (glutamic acid-alanine-glutamic acid-alanine) was cloned, which is processed (sequentially cleaved) by the aminopeptidyl peptidase.

Transformation into Yeast Strain

Plasmids were linearized (cut) within their promoter sequence using SacI/BamHI/another restriction enzyme, depending on the promoter used. The linearized plasmids were transformed individually into the GS 115 strain of the yeast, Pichia pastoris. Following transformation, the yeast cells was plated onto YPD (Yeast Extract-Peptone-Dextrose) plates containing geneticin (G418≥0.25 mg/ml) to enable ‘antibiotic resistance’ based selection of the transformed yeast colonies. The successful integration of the respective GOI was subsequently verified using colony PCR. FIG. 1 schematically illustrates the construct containing the GOI and its integration within the yeast genome.

Colony screening and growth experiment with the shaking flasks Selected clones (colonies) were streaked from the transformation plates into fresh YPD plates containing geneticin (G418≥0.25 mg/ml) to obtain monoclonal colonies. Each monoclonal colony was then subjected to serial plating onto YPD plates with increasing geneticin concentration (up to 3 mg/ml) to obtain a set of clones with variable gene copy number with respect to the gene of interest. Subsequently, at least 10 such monoclonal colonies were chosen to be individually tested in shake flask studies. Note that glycerol stocks were also made for these selected monoclonal colonies using standard procedure to enable long term storage at −80° C.

Shake flask studies were initiated with at least 10 monoclonal colonies for each new construct in parallel with appropriate reference control(s). Each individual colony (grown as a biomass on YPD plates) was inoculated into a shake flask containing 10 mls of BMGY (Buffered Glycerol-complex) medium and incubated for 24 hours at 30° C., with shaking at 270 rpm. After 24-hours the cultures were induced for 3 consecutive days with a daily dose of either a final concentration of 0.2% potassium formate+0.5-1% sorbitol or 0.25-1% methanol. In some cases, a daily double dose of 0.2% potassium formate+0.5-1% sorbitol was fed to the cultures.

### Enzyme Assay

At the end of 70-72 hours, shake flask cultures of cells were subjected to centrifugation at 10,000 g for five minutes. The supernatant was collected and the activity of the enzyme in the supernatant was measured using an HPLC based enzyme assay. In one embodiment, the activity of the enzyme in the supernatant was measured by setting up the reaction mixture that included 125 μl of 200 mM sodium citrate buffer (pH 4.8), 50-100 μl of the supernatant and 25 μl of a 554 μM solution of Cannabigerolic acid (CBGA) in DMSO. Note that the substrate (e.g., CBGA) concentration used in the assay can vary from 554 μM-2.7 mM.

Following incubation at 30° C. for 30 mins, the reaction was stopped by the addition of 250 μl of 100% methanol (1:1 MeOH: vol. of reaction mixture). The stopped reaction was then centrifuged at 14000 rpm for 15 mins and the supernatant analyzed by HPLC. In one embodiment the supernatant comprising the enzyme is incubated with a substrate, for example, CBGA or CBGVA for 1-16 hours, preferably for 1-15 hours, preferably for 1-14 hours, preferably for 1-13 hours, preferably for 1-12 hours, preferably for 1-11 hours, preferably for 1-10 hours, preferably for 1-9 hours, preferably for 1-8 hours, preferably for 1-7 hours, preferably for 1-6 hours, preferably for 1-5 hours, preferably for 1-4 hours, preferably for 1-3 hours, or preferably for 1-2 hours.

### HPLC

HPLC was performed on the Agilent 1260 Infinity II system. Mobile Phase A refers to water with 0.1% formic acid while the mobile Phase B refers to acetonitrile with 0.1% formic acid. Each new run on the HPLC starts with a one-hour equilibration at 25% A and 75% B. To ensure that the system was thoroughly cleaned and there was no carryover, prior to the analysis of a new set of samples the column was cleaned by injecting pure methanol followed by an injection of DMSO. Such a cleaning routine was carried out once every 20 samples. The injection volume was set at 20 μl and the flow rate of the mobile phase was set at 400 μl/min. Samples were analyzed using the following gradient:
0.0 min 25% A, 75% B,
5.00 min: 4% A, 96% B,
5.01 min: 0% A, 100% B, and
6.00 min: 0% A, 100% B.

10 μl of 0.01 mg/mL of Δ8-THC (Cerilliant T-032 at 1 mg/mL diluted with 100% methanol) was added as an internal standard to each sample and mixed five times within the injector compartment prior to loading onto the column. Calibration curves for CBGA (Cerilliant C-142 at 1 mg/mL diluted with 100% methanol), CBDA (Cerilliant C-144 at 1 mg/mL diluted with 100% methanol), and Δ9-THCA-A (Cerilliant T-093 at 1 mg/mL diluted with 100% methanol) were generated separately and were used to calculate the amount of CBGA, CBDA and/or Δ9-THCA-A produced in a test sample by an enzyme of the present disclosure. Prior to, and following each injection, the injection needle was washed for 3 seconds with 100% MeOH. The column chamber was set to a temperature of 30.0±0.8° C. while the Diode Array Detector (DAD) was set to scan from 190 nm to 400 nm with data primarily collected at 269 nm with a peak width of 5 Hz (>0.05 min). Data was analyzed using Agilent OpenLab CDS running on Windows 10.

The sequences of this disclosure are provided with the Reference sequence numbered as SEQ ID NO: 1 or 71.

Table 1 shows enzyme assay data.

TABLE 1

| Enzyme activity | |
|---|---|
| relative to SEQ | |
| ID 71 (Reference) | SEQ ID NOs: |
| ≥5-fold | 75, 76, 78, 79, 80, 83, 84, 86, 87, 92, 93, 97, 98, 99 |
| <5-fold | 72, 73, 74, 77, 81, 82, 85, 88-91, 94, 95, 96, 100, 101 |
| Not determined | 102-115 |

Table 2 correlates the SEQ ID numbers of the polynucleotide sequences for various accessory elements used to produce the CBDA synthase mutants according to this disclosure to the SEQ ID numbers of their corresponding polypeptide (protein) sequences.

TABLE 2

| | Corresponding | |
|---|---|---|
| Nucleotide | Amino Acid | |
| SEQ ID No. | SEQ ID No. | Accessory Element |
| SEQ ID No. 46 | SEQ ID No. 116 | Secretion peptide alpha-MF (alpha mating factor from S. cerevisiae |
| | | yeast) |
| SEQ ID No. 47 | SEQ ID No. 117 | Secretion peptide HSP150 (Heat shock protein 150 from P. pastoris |
| | | yeast) |
| SEQ ID No. 48 | SEQ ID No. 118 | Secretion peptide: combination of pre region of alpha -MF (19 |
| | | amino acids from alpha -MF) and pro-region of HSP150 from P. |
| | | pastoris |
| SEQ ID No. 49 | SEQ ID No. 119 | Secretion peptide: combination of pre region of alpha -MF and |
| | | double repeat of pro-region of HSP150 |
| SEQ ID No. 50 | SEQ ID No. 120 | Secretion peptide SUC2 (invertase from S. cerevisiae) |
| SEQ ID No. 51 | SEQ ID No. 121 | Secretion peptide PHO1 (acid phosphatase from S. cerevisiae) |
| SEQ ID No. 52 | SEQ ID No. 122 | Secretion peptide PIR1 (55 amino acids from Protein with internal |
| | | repeats 1, from P. pastoris |
| SEQ ID No. 53 | SEQ ID No. 123 | Secretion peptide OST1 (dolichyl-diphosphooligosaccharide protein |
| | | glycotransferase subunit OST1) |
| SEQ ID No. 54 | SEQ ID No. 124 | Secretion peptide OST1 fused to pro-region of alpha -MF secretion |
| | | peptide (20-85 amino acids of alpha -MF secretion peptide) |
| SEQ ID No. 55 | SEQ ID No. 125 | Secretion peptide DDDK (first 18 amino acids from protein with |
| | | access number AOA68487.1 from P. pastoris) |
| SEQ ID No. 56 | SEQ ID No. 126 | Human influenza hemagglutinin (HA) tag |
| or 129 | | |
| SEQ ID No. 57 | SEQ ID No. 127 | 6X Histidine tag |
| SEQ ID No. 58 | SEQ ID No. 128 | HN tag |
| SEQ ID No. 59 | Not applicable | Promoter pAOX1 (alcohol oxidase 1) |
| SEQ ID No. 60 | Not applicable | Promoter pAOX2 (alcohol oxidase 2) |
| SEQ ID No. 61 | Not applicable | Promoter pFDH (NAD⁺-dependent formylglutathione |
| | | dehydrogenase) |
| SEQ ID No. 62 | Not applicable | Promoter pPEX14 (peroxin Pex14p) |
| SEQ ID No. 63 | Not applicable | Promoter pFGH (S-hydroxymethyl-glutathione hydrolase) |
| SEQ ID No. 64 | Not applicable | Promoter pDAK (dihydroxyacetone kinase) |
| SEQ ID No. 65 | Not applicable | Promoter pFBA (fructose 1,6-bisphosphate aldolase) |
| SEQ ID No. 66 | Not applicable | Promoter pPEX5 (peroxisomal membrane signal receptor PTS1) |
| SEQ ID No. 67 | Not applicable | Promoter pADH2 (alcohol dehydrogenase 2) |
| SEQ ID No. 68 | Not applicable | Promoter pPEX8 (peroxisomal protein Pex8p) |
| SEQ ID No. 69 | Not applicable | Plasmid pPIC 3.5 |
| SEQ ID No. 70 | Not applicable | Plasmid pL |

(ORIGINAL/REFERENCE CBDAS)
SEQ ID NO: 1
AACCCAAGAGAAAACTTCTTGAAGTGTTTTTCCCAGTACATCCCAAACAACGCTACAAACTTGAAGTTGGTTTAC
ACTCAGAACAACCCATTGTACATGTCCGTTTTGAACTCCACAATCCACAACTTGAGATTCACTTCCGACACTACT
CCAAAGCCATTGGTTATCGTTACTCCATCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCTTTCGTT
ATCGTTGACTTGAGAAACATGAGATCCATCAAGATCGACGTTCACTCCCAGACTGCTTGGGTTGAAGCTGGTGCT
ACTTTGGGTGAAGTTTACTACTGGGTTAACGAGAAGAACGAGAACTTGTCCTTGGCTGCTGGTTACTGTCCAACT
GTTTGTGCTGGTGGTCATTTCGGTGGTGGTGGTTATGGTCCATTGATGAGAAACTACGGTTTGGCTGCTGACAAC
ATCATCGACGCTCACTTGGTTAACGTTCACGGTAAGGTTTTGGACAGAAAGTCCATGGGTGAGGACTTGTTCTGG
GCTTTGAGAGGTGGTGGTGCTGAATCCTTCGGTATTATCGTTGCTTGGAAGATCAGATTGGTTGCTGTTCCAAAG
TCCACTATGTTCTCCGTTAAGAAAATCATGGAAATCCACGAATTGGTTAAGTTGGTTAACAAGTGGCAGAACATT
GCTTACAAGTACGACAAGGATTTGTTGTTGATGACTCACTTCATCACTAGAAACATCACTGACAACCAGGGTAAG
AACAAGACTGCTATCCACACTTACTTCTCTTCCGTTTTCTTGGGTGGTGTTGACTCCTTGGTTGATTTGATGAAC
AAGTCCTTCCCAGAGTTGGGTATCAAGAAAACTGACTGTAGACAGTTGTCCTGGATCGACACTATCATCTTCTAC
TCCGGTGTTGTTAACTACGACACAGACAACTTCAACAAAGAGATCTTGTTGGACAGATCCGCTGGACAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGTATGTACGCTTTGTACCCATACGGTGGTATCATGGACGAAATTTCCGAGTCC
GCTATTCCATTCCCACACAGAGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAGAACCCAAGATTG
GCTTACTTGAACTACAGAGACTTGGACATCGGAATCAACGACCCAAAGAACCCTAACAACTACACTCAGGCTAGA
ATCTGGGGTGAAAAGTACTTCGGTAAGAACTTCGACAGATTGGTTAAGGTTAAGACTTTGGTTGACCCAAACAAT
TTCTTCAGAAACGAGCAGTCCATCCCTCCATTGCCAAGACATAGACATTAA
SEQ ID NO: 2
AACCCAAGAGAAAACTTCTTGAAGTGTTTTTCCCAGTACATCCCAAACAACGCTACAAACTTGAAGTTGGTTTAC
ACTCAGAACAACCCATTGTACATGTCCGTTTTGAACTCCACAATCCACAACTTGAGATTCACTTCCGACACTACT
CCAAAGCCATTGGTTATCGTTACTCCATCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCTTTCGTT
ATCGTTGACTTGAGAAACATGAGATCCATCAAGATCGACGTTCACTCCCAGACTGCTTGGGTTGAAGCTGGTGCT
ACTTTGGGTGAAGTTTACTACTGGGTTAACGAGAAGAACGAGAACTTGTCCTTGGCTGCTGGTTACTGTCCAACT
GTTGGTGCTGGTGGTCATTTCGGTGGTGGTGGTTATGGTCCATTGATGAGAAACTACGGTTTGGCTGCTGACAAC
ATCATCGACGCTCACTTGGTTAACGTTCACGGTAAGGTTTTGGACAGAAAGTCCATGGGTGAGGACTTGTTCTGG
GCTTTGAGAGGTGGTGGTGCTGAATCCTTCGGTATTATCGTTGCTTGGAAGATCAGATTGGTTGCTGTTCCAAAG
TCCACTATGTTCTCCGTTAAGAAAATCATGGAAATCCACGAATTGGTTAAGTTGGTTAACAAGTGGCAGAACATT
GCTTACAAGTACGACAAGGATTTGTTGTTGATGACTCACTTCATCACTAGAAACATCACTGACAACCAGGGTAAG
AACAAGACTGCTATCCACACTTACTTCTCTTCCGTTTTCTTGGGTGGTGTTGACTCCTTGGTTGATTTGATGAAC
AAGTCCTTCCCAGAGTTGGGTATCAAGAAAACTGACTGTAGACAGTTGTCCTGGATCGACACTATCATCTTCTAC
TCCGGTGTTGTTAACTACGACACAGACAACTTCAACAAAGAGATCTTGTTGGACAGATCCGCTGGACAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGTATGTACGCTTTGTACCCATACGGTGGTATCATGGACGAAATTTCCGAGTCC
GCTATTCCATTCCCACACAGAGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAGAACCCAAGATTG
GCTTACTTGAACTACAGAGACTTGGACATCGGAATCAACGACCCAAAGAACCCTAACAACTACACTCAGGCTAGA
ATCTGGGGTGAAAAGTACTTCGGTAAGAACTTCGACAGATTGGTTAAGGTTAAGACTTTGGTTGACCCAAACAAT
TTCTTCAGAAACGAGCAGTCCATCCCTCCATTGCCAAGACATAGACATTAA
SEQ ID NO: 3
AACCCAAGAGAAAACTTCTTGAAGTGTTTTTCCCAGTACATCCCAAACAACGCTACAAACTTGAAGTTGGTTTAC
ACTCAGAACAACCCATTGTACATGTCCGTTTTGAACTCCACAATCCACAACTTGAGATTCACTTCCGACACTACT
CCAAAGCCATTGGTTATCGTTACTCCATCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCTTTCGTT
ATCGTTGACTTGAGAAACATGAGATCCATCAAGATCGACGTTCACTCCCAGACTGCTTGGGTTGAAGCTGGTGCT
ACTTTGGGTGAAGTTTACTACTGGGTTAACGAGAAGAACGAGAACTTGTCCTTGGCTGCTGGTTACTGTCCAACT
GTTTGTGCTGGTGGTCATTTCGGTGGTGGTGGTTATGGTCCATTGATGAGAAACTACGGTTTGGCTGCTGACAAC
ATCATCGACGCTCACTTGGTTAACGTTCACGGTAAGGTTTTGGACAGAAAGTCCATGGGTGAGGACTTGTTCTGG
GCTTTGAGAGGTGGTGGTGCTGAATCCTTCGGTATTATCGTTGCTTGGAAGATCAGATTGGTTGCTGTTCCAAAG
TCCACTATGTTCTCCGTTAAGAAAATCATGGAAATCCACGAATTGGTTAAGTTGGTTAACAAGTGGCAGAACATT
GCTTACAAGTACGACAAGGATTTGTTGTTGATGACTCACTTCATCACTAGAAACATCACTGACAACCAGGGTAAG
AACAAGACTGCTATCCACACTTACTTCTCTTCCGTTTTCTTGGGTGGTGTTGACTCCTTGGTTGATTTGATGAAC
AAGTCCTTCCCAGAGTTGGGTATCAAGAAAACTGACTGTAGACAGTTGTCCTGGATCGACACTATCATCTTCTAC
TCCGGTGTTGTTAACTACGACACAGACAACTTCAACAAAGAGATCTTGTTGGACAGATCCGCTGGACAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGTATGTACGCTTTGTACCCATACGGTGGTATCATGGACGAAATTTCCGAGTCC
GCTATTCCATTCCCACACAGAGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCCAGAACCCAAGATTG
GCTTACTTGAACTACAGAGACTTGGACATCGGAATCAACGACCCAAAGAACCCTAACAACTACACTCAGGCTAGA
ATCTGGGGTGAAAAGTACTTCGGTAAGAACTTCGACAGATTGGTTAAGGTTAAGACTTTGGTTGACCCAAACAAT
TTCTTCAGAAACGAGCAGTCCATCCCTCCATTGCCAAGACATAGACATTAA
SEQ ID NO: 4
AACCCAAGAGAAAACTTCTTGAAGTGTTTTTCCCAGTACATCCCAAACAACGCTACAAACTTGAAGTTGGTTTAC
ACTCAGAACAACCCATTGTACATGTCCGTTTTGAACTCCACAATCCACAACTTGAGATTCATTTCCGACACTACT
CCAAAGCCATTGGTTATCGTTACTCCATCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCTTTCGTT
ATCGTTGACTTGAGAAACATGAGATCCATCAAGATCGACGTTCACTCCCAGACTGCTTGGGTTGAAGCTGGTGCT
ACTTTGGGTGAAGTTTACTACTGGGTTAACGAGAAGAACGAGAACTTGTCCTTGGCTGCTGGTTACTGTCCAACT
GTTTGTGCTGGTGGTCATTTCGGTGGTGGTGGTTATGGTCCATTGATGAGAAACTACGGTTTGGCTGCTGACAAC
ATCATCGACGCTCACTTGGTTAACGTTCACGGTAAGGTTTTGGACAGAAAGTCCATGGGTGAGGACTTGTTCTGG
GCTTTGAGAGGTGGTGGTGCTGAATCCTTCGGTATTATCGTTGCTTGGAAGATCAGATTGGTTGCTGTTCCAAAG
TCCACTATGTTCTCCGTTAAGAAAATCATGGAAATCCACGAATTGGTTAAGTTGGTTAACAAGTGGCAGAACATT
GCTTACAAGTACGACAAGGATTTGTTGTTGATGACTCACTTCATCACTAGAAACATCACTGACAACCAGGGTAAG
AACAAGACTGCTATCCACACTTACTTCTCTTCCGTTTTCTTGGGTGGTGTTGACTCCTTGGTTGATTTGATGAAC
AAGTCCTTCCCAGAGTTGGGTATCAAGAAAACTGACTGTAGACAGTTGTCCTGGATCGACACTATCATCTTCTAC
TCCGGTGTTGTTAACTACGACACAGACAACTTCAACAAAGAGATCTTGTTGGACAGATCCGCTGGACAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGTATGTACGCTTTGTACCCATACGGTGGTATCATGGACGAAATTTCCGAGTCC
GCTATTCCATTCCCACACAGAGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCCAGAACCCAAGATTG
GCTTACTTGAACTACAGAGACTTGGACATCGGAATCAACGACCCAAAGAACCCTAACAACTACACTCAGGCTAGA
ATCTGGGGTGAAAAGTACTTCGGTAAGAACTTCGACAGATTGGTTAAGGTTAAGACTTTGGTTGACCCAAACAAT
TTCTTCAGAAACGAGCAGTCCATCCCTCCATTGCCAAGACATAGACATTAA
SEQ ID NO: 5
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 6
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAAGCGGTGCT
ACATTGGGTGAAGTTTATTACTGGGTTAACGAGAAGAACGAGAACCTCTCCTTGGCCGCTGGTTACTGTCCAACC
GTTGGTGCTGGTGGTCATTTCTCTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGGTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 7
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAAGCGGTGCT
ACATTGGGTGAAGTTTATTACTGGGTTAACGAGAAGAACGAGAACCTCTCCTTGGCCGCTGGTTACTGTCCAACC
GTTGGTGCTGGTGGTCATTTCTCTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGGTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTAAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 8
AACGCAAGAGAAAACTTCTTGAAGTGTTTTTCCAAGCACATCCCCAACAACGTTGCTAACCCTAAGTTGGTATAC
ACTCAGCACGACCAGTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 9
AACGCAAGAGAAAACTTCTTGAAGTGTTTTTCCCAGCACATCGGCAACAGGGGTAATGTATACACTCAGCACGAC
CAGTTGTACATGTCCGTTTTGAACTCCACAATCCACAACTTGAGATTCACATCCGACACTACTCCAAAGCCCTTG
GTTATCGTTACTCCCTCCAACGTTGCCCACATCCAGGCTACTATTTTGTGTTCCAAGAAGGTTGGATTGCAGATC
AGAACAAGATCCGGTGGTCACGACTCTGAAGGTATGTCCTACATTTCCCAGGTTCCCTTCGTTATCGTTGACCTT
AGAAACATGCGTTCCATCAAGATCGACGTTCACTCCCAGACTGCTTGGGTTGAAGCAGGTGCTACGTTGGGTGAA
GTTTATTACTGGGTTAACGAGAAGAACGAGAACCTCTCCTTGGCCGCTGGTTACTGTCCAACCGTTGGTGCTGGT
GGTCATTTCGGTGGAGGTGGTTATGGTCCATTGATGAGAAACTACGGACTTGCTGCTGACAACATCATCGACGCT
CACTTGGTTAACGTTCACGGTAAGGTTTTGGACAGAAAGTCCATGGGAGAGGACTTGTTCTGGGCTTTGCGTGGT
GGTGGTGCTGAATCCTTCGGTATTATCGTTGCTTGGAAGATCAGACTTGTTGCTGTTCCCAAGTCCACTATGTTC
TCCGTTAAGAAAATCATGGAAATCCACGAATTGGTTAAGTTGGTTAACAAGTGGCAGAACATTGCTTACAAGTAC
GACAAGGACTTGTTGTTGATGACTCACTTCATCACTAGAAACATCACTGACAACCAGGGTAAGAACAAAACTGCT
ATCCACACTTACTTCTCTTCCGTTTTCTTGGGTGGTGTTGACTCCTTGGTTGATTTGATGAACAAGTCCTTCCCA
GAGTTGGGCATCAAGAAAACTGACTGTAGACAGTTGTCCTGGATCGACACTATCATCTTCTACTCCGGTGTTGTA
AACTACGACACAGACAACTTCAACAAAGAGATCTTGTTGGACAGATCCGCTGGCCAGAACGGTGCTTTCAAGATC
AAGTTGGACTACGTTAAGAAGCCAATCCCAGAGTCCGTTTTCGTTCAGATTTTGGAGAAGTTGTACGAAGAGGAC
ATCGGTGCTGGAATGTACGCTTTGTACCCTTACGGTGGCATCATGGACGAAATCTCCGAGTCCGCTATTCCCTTC
CCACACCGTGCTGGTATCTTGTACGAGTTGTGGTACATCTGTTCTTGGGAGAAGCAAGAGGACAACGAGAAGCAC
TTGAACTGGATCAGAAACATCTACAACTTCATGACTCCATACGTTTCCAAAAACCCTAGATTGGCATACCTAAAC
TACAGAGACTTGGACATCGGCATCAACGACCCCAAGAACCCTAACAACTACACTCAGGCTAGAATCTGGGGTGAA
AAGTACTTCGGTAAGAACTTCGACAGATTGGTTAAGGTTAAGACTTTGGTTGACCCTAACAATTTCTTCAGGAAC
GAGCAGTCCATCCCTCCATTGCCAAGACATAGACATTAA
SEQ ID NO: 10
AACGCAAGAGAAAACTTCTTGAAGTGTTTTTCCCAGCACATCCCCAACGCTAACCCTAAGTTGGTATACACTCAG
CACGACCAGTTGTACATGTCCGTTTTGAACTCCACAATCCACAACTTGAGATTCACATCCGACACTACTCCAAAG
CCCTTGGTTATCGTTACTCCCTCCAACGTTGCCCACATCCAGGCTACTATTTTGTGTTCCAAGAAGGTTGGATTG
CAGATCAGAACAAGATCCGGTGGTCACGACTCTGAAGGTATGTCCTACATTTCCCAGGTTCCCTTCGTTATCGTT
GACCTTAGAAACATGCGTTCCATCAAGATCGACGTTCACTCCCAGACTGCTTGGGTTGAAGCAGGTGCTACGTTG
GGTGAAGTTTATTACTGGGTTAACGAGAAGAACGAGAACCTCTCCTTGGCCGCTGGTTACTGTCCAACCGTTGGT
GCTGGTGGTCATTTCGGTGGAGGTGGTTATGGTCCATTGATGAGAAACTACGGACTTGCTGCTGACAACATCATC
GACGCTCACTTGGTTAACGTTCACGGTAAGGTTTTGGACAGAAAGTCCATGGGAGAGGACTTGTTCTGGGCTTTG
CGTGGTGGTGGTGCTGAATCCTTCGGTATTATCGTTGCTTGGAAGATCAGACTTGTTGCTGTTCCCAAGTCCACT
ATGTTCTCCGTTAAGAAAATCATGGAAATCCACGAATTGGTTAAGTTGGTTAACAAGTGGCAGAACATTGCTTAC
AAGTACGACAAGGACTTGTTGTTGATGACTCACTTCATCACTAGAAACATCACTGACAACCAGGGTAAGAACAAA
ACTGCTATCCACACTTACTTCTCTTCCGTTTTCTTGGGTGGTGTTGACTCCTTGGTTGATTTGATGAACAAGTCC
TTCCCAGAGTTGGGCATCAAGAAAACTGACTGTAGACAGTTGTCCTGGATCGACACTATCATCTTCTACTCCGGT
GTTGTAAACTACGACACAGACAACTTCAACAAAGAGATCTTGTTGGACAGATCCGCTGGCCAGAACGGTGCTTTC
AAGATCAAGTTGGACTACGTTAAGAAGCCAATCCCAGAGTCCGTTTTCGTTCAGATTTTGGAGAAGTTGTACGAA
GAGGACATCGGTGCTGGAATGTACGCTTTGTACCCTTACGGTGGCATCATGGACGAAATCTCCGAGTCCGCTATT
CCCTTCCCACACCGTGCTGGTATCTTGTACGAGTTGTGGTACATCTGTTCTTGGGAGAAGCAAGAGGACAACGAG
AAGCACTTGAACTGGATCAGAAACATCTACAACTTCATGACTCCATACGTTTCCAAAAACCCTAGATTGGCATAC
CTAAACTACAGAGACTTGGACATCGGCATCAACGACCCCAAGAACCCTAACAACTACACTCAGGCTAGAATCTGG
GGTGAAAAGTACTTCGGTAAGAACTTCGACAGATTGGTTAAGGTTAAGACTTTGGTTGACCCTAACAATTTCTTC
AGGAACGAGCAGTCCATCCCTCCATTGCCAAGACATAGACATTAA
SEQ ID NO: 11
AACGCAAGAGAAAACTTGTTGAAGTGTTTGTCCCAGCACGGTGACGGCACAGGAAAGACAGTATACACTCAGAAT
AATCCCTTGTACATGTCCGTTTTGAACTCCACAATCCACAACTTGAGATTCACATCCGACACTACTCCAAAGCCC
TTGGTTATCGTTACTCCCTCCAACGTTGCCAAGATCCAGGCTACTATTTTGTGTTCCAAGAAGGTTGGATTGCAG
ATCAGAACAAGATCCGGTGGTCACGACTCTGAAGGTATGTCCTACATTTCCCAGGTTCCCTTCGTTATCGTTGAC
CTTAGAAACATGCGTTCCATCAAGATCGACGTTCACTCCCAGACTGCTTGGGTTGAAGCAGGTGCTACGTTGGGT
GAAGTTTATTACTGGGTTAACGAGAAGAACGAGAACCTCTCCTTGGCCGCTGGTTACTGTCCAACCGTTGGTGCT
GGTGGTCATTTCGGTGGAGGTGGTTATGGTCCATTGATGAGAAACTACGGACTTGCTGCTGACAACATCATCGAC
GCTCACTTGGTTAACGTTCACGGTAAGGTTTTGGACAGAAAGTCCATGGGAGAGGACTTGTTCTGGGCTTTGCGT
GGTGGTGGTGCTGAATCCTTCGGTATTATCGTTGCTTGGAAGATCAGACTTGTTGCTGTTCCCAAGTCCACTATG
TTCTCCGTTAAGAAAATCATGGAAATCCACGAATTGGTTAAGTTGGTTAACAAGTGGCAGAACATTGCTTACAAG
TACGACAAGGACTTGTTGTTGATGACTCACTTCATCACTAGAAACATCACTGACAACCAGGGTAAGAACAAAACT
GCTATCCACACTTACTTCTCTTCCGTTTTCTTGGGTGGTGTTGACTCCTTGGTTGATTTGATGAACAAGTCCTTC
CCAGAGTTGGGCATCAAGAAAACTGACTGTAGACAGTTGTCCTGGATCGACACTATCATCTTCTACTCCGGTGTT
GTAAACTACGACACAGACAACTTCAACAAAGAGATCTTGTTGGACAGATCCGCTGGCCAGAACGGTGCTTTCAAG
ATCAAGTTGGACTACGTTAAGAAGCCAATCCCAGAGTCCGTTTTCGTTCAGATTTTGGAGAAGTTGTACGAAGAG
GACATCGGTGCTGGAATGTACGCTTTGTACCCTTACGGTGGCATCATGGACGAAATCTCCGAGTCCGCTATTCCC
TTCCCACACCGTGCTGGTATCTTGTACGAGTTGTGGTACATCTGTTCTTGGGAGAAGCAAGAGGACAACGAGAAG
CACTTGAACTGGATCAGAAACATCTACAACTTCATGACTCCATACGTTTCCAAAAACCCTAGATTGGCATACCTA
AACTACAGAGACTTGGACATCGGCATCAACGACCCCAAGAACCCTAACAACTACACTCAGGCTAGAATCTGGGGT
GAAAAGTACTTCGGTAAGAACTTCGACAGATTGGTTAAGGTTAAGACTTTGGTTGACCCTAACAATTTCTTCAGG
AACGAGCAGTCCATCCCTCCATTGCCAAGACATAGACATTAA
SEQ ID NO: 12
AACGCAAGAGAAAACTTGTTGAAGTGTTTGTCCCAGCACGACGGCACAGGAAAGACAGTATACACTCAGAATAAT
CCCTTGTACATGTCCGTTTTGAACTCCACAATCCACAACTTGAGATTCACATCCGACACTACTCCAAAGCCCTTG
GTTATCGTTACTCCCTCCAACGTTGCCAAGATCCAGGCTACTATTTTGTGTTCCAAGAAGGTTGGATTGCAGATC
AGAACAAGATCCGGTGGTCACGACTCTGAAGGTATGTCCTACATTTCCCAGGTTCCCTTCGTTATCGTTGACCTT
AGAAACATGCGTTCCATCAAGATCGACGTTCACTCCCAGACTGCTTGGGTTGAAGCAGGTGCTACGTTGGGTGAA
GTTTATTACTGGGTTAACGAGAAGAACGAGAACCTCTCCTTGGCCGCTGGTTACTGTCCAACCGTTGGTGCTGGT
GGTCATTTCGGTGGAGGTGGTTATGGTCCATTGATGAGAAACTACGGACTTGCTGCTGACAACATCATCGACGCT
CACTTGGTTAACGTTCACGGTAAGGTTTTGGACAGAAAGTCCATGGGAGAGGACTTGTTCTGGGCTTTGCGTGGT
GGTGGTGCTGAATCCTTCGGTATTATCGTTGCTTGGAAGATCAGACTTGTTGCTGTTCCCAAGTCCACTATGTTC
TCCGTTAAGAAAATCATGGAAATCCACGAATTGGTTAAGTTGGTTAACAAGTGGCAGAACATTGCTTACAAGTAC
GACAAGGACTTGTTGTTGATGACTCACTTCATCACTAGAAACATCACTGACAACCAGGGTAAGAACAAAACTGCT
ATCCACACTTACTTCTCTTCCGTTTTCTTGGGTGGTGTTGACTCCTTGGTTGATTTGATGAACAAGTCCTTCCCA
GAGTTGGGCATCAAGAAAACTGACTGTAGACAGTTGTCCTGGATCGACACTATCATCTTCTACTCCGGTGTTGTA
AACTACGACACAGACAACTTCAACAAAGAGATCTTGTTGGACAGATCCGCTGGCCAGAACGGTGCTTTCAAGATC
AAGTTGGACTACGTTAAGAAGCCAATCCCAGAGTCCGTTTTCGTTCAGATTTTGGAGAAGTTGTACGAAGAGGAC
ATCGGTGCTGGAATGTACGCTTTGTACCCTTACGGTGGCATCATGGACGAAATCTCCGAGTCCGCTATTCCCTTC
CCACACCGTGCTGGTATCTTGTACGAGTTGTGGTACATCTGTTCTTGGGAGAAGCAAGAGGACAACGAGAAGCAC
TTGAACTGGATCAGAAACATCTACAACTTCATGACTCCATACGTTTCCAAAAACCCTAGATTGGCATACCTAAAC
TACAGAGACTTGGACATCGGCATCAACGACCCCAAGAACCCTAACAACTACACTCAGGCTAGAATCTGGGGTGAA
AAGTACTTCGGTAAGAACTTCGACAGATTGGTTAAGGTTAAGACTTTGGTTGACCCTAACAATTTCTTCAGGAAC
GAGCAGTCCATCCCTCCATTGCCAAGACATAGACATTAA
SEQ ID NO: 13
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 14
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 15
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTCTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 16
AACGCAAGAGAAAACTTCTTGAAGTGTTTTTCCCAGCACATCCCCAACGATGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTTCCTGGTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 17
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGGACCTCTCCTTTCCTGGTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCTCAACTATGTTCTCCGTTAAGAAAATCATGGAAATCCACGAATTGGTTAAGTTGGTTAACAAGTGGCAGAAC
ATTGCTTACAAGTACGACAAGGACTTGTTGTTGATGACTCACTTCATCACTAGAAACATCACTGACAACCAGGGT
AAGAACAAAACTGCTATCCACACTTACTTCTCTTCCGTTTTCTTGGGTGGTGTTGACTCCTTGGTTGATTTGATG
AACAAGTCCTTCCCAGAGTTGGGCATCAAGAAAACTGACTGTAGACAGTTGTCCTGGATCGACACTATCATCTTC
TACTCCGGTGTTGTAAACTACGACACAGACAACTTCAACAAAGAGATCTTGTTGGACAGATCCGCTGGCCAGAAC
GGTGCTTTCAAGATCAAGTTGGACTACGTTAAGAAGCCAATCCCAGAGTCCGTTTTCGTTCAGATTTTGGAGAAG
TTGTACGAAGAGGACATCGGTGCTGGAATGTACGCTTTGTACCCTTACGGTGGCATCATGGACGAAATCTCCGAG
TCCGCTATTCCCTTCCCACACCGTGCTGGTATCTTGTACGAGTTGTGGTACATCTGTTCTTGGGAGAAGCAAGAG
GACAACGAGAAGCACTTGAACTGGATCAGAAACATCTACAACTTCATGACTCCATACGTTTCCAAAAACCCTAGA
TTGGCATACCTAAACTACAGAGACTTGGACATCGGCATCAACGACCCCAAGAACCCTAACAACTACACTCAGGCT
AGAATCTGGGGTGAAAAGTACTTCGGTAAGAACTTCGACAGATTGGTTAAGGTTAAGACTTTGGTTGACCCTAAC
AATTTCTTCAGGAACGAGCAGTCCATCCCTCCATTGCCAAGACATAGACATTAA
SEQ ID NO: 18
AACGCAAGAGAAAACTTCTTGAAGTGTTTTTCCCAGCACATCCCCAACGATGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAAAATTTCGGTATTATCGTTGCTTGGAAGATCAGACTTGTTGCTGTTCCCAAG
TCCACTATGTTCTCCGTTAAGAAAAATATGGAAATCCACGCTTTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACGTTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 19
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGAGGCACAGGAAGTGGAACCTTCAAGAAAGAGATCTTGTTGGACAGATCCGCTGGCCAGAACGGTGCTTTC
AAGATCAAGTTGGACTACGTTAAGAAGCCAATCCCAGAGTCCGTTTTCGTTCAGATTTTGGAGAAGTTGTACGAA
GAGGACATCGGTGCTGGAATGTACGCTTTGTACCCTTACGGTGGCATCATGGACGAAATCTCCGAGTCCGCTATT
CCCTTCCCACACCGTGCTGGTATCTTGTACGAGTTGTGGTACATCTGTTCTTGGGAGAAGCAAGAGGACAACGAG
AAGCACTTGAACTGGATCAGAAACATCTACAACTTCATGACTCCATACGTTTCCAAAAACCCTAGATTGGCATAC
CTAAACTACAGAGACTTGGACATCGGCATCAACGACCCCAAGAACCCTAACAACTACACTCAGGCTAGAATCTGG
GGTGAAAAGTACTTCGGTAAGAACTTCGACAGATTGGTTAAGGTTAAGACTTTGGTTGACCCTAACAATTTCTTC
AGGAACGAGCAGTCCATCCCTCCATTGCCAAGACATAGACATTAA
SEQ ID NO: 20
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGAGGCACAGGAAGTGGAACCAAGAAAGAGATCTTGTTGGACAGATCCGCTGGCCAGAACGGTGCTTTCAAG
ATCAAGTTGGACTACGTTAAGAAGCCAATCCCAGAGTCCGTTTTCGTTCAGATTTTGGAGAAGTTGTACGAAGAG
GACATCGGTGCTGGAATGTACGCTTTGTACCCTTACGGTGGCATCATGGACGAAATCTCCGAGTCCGCTATTCCC
TTCCCACACCGTGCTGGTATCTTGTACGAGTTGTGGTACATCTGTTCTTGGGAGAAGCAAGAGGACAACGAGAAG
CACTTGAACTGGATCAGAAACATCTACAACTTCATGACTCCATACGTTTCCAAAAACCCTAGATTGGCATACCTA
AACTACAGAGACTTGGACATCGGCATCAACGACCCCAAGAACCCTAACAACTACACTCAGGCTAGAATCTGGGGT
GAAAAGTACTTCGGTAAGAACTTCGACAGATTGGTTAAGGTTAAGACTTTGGTTGACCCTAACAATTTCTTCAGG
AACGAGCAGTCCATCCCTCCATTGCCAAGACATAGACATTAA
SEQ ID NO: 21
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGGCGATGGAACAGGTAAGAAAGAGATCTTGTTGGACAGATCCGCTAAGGGCAACGGTGCTTTCAAGATC
AAGTTGGACTACGTTAAGAAGCCAATCCCAGAGTCCGTTTTCGTTCAGATTTTGGAGAAGTTGTACGAAGAGGAC
ATCGGTGCTGGAATGTACGCTTTGTACCCTTACGGTGGCATCATGGACGAAATCTCCGAGTCCGCTATTCCCTTC
CCACACCGTGCTGGTATCTTGTACGAGTTGTGGTACATCTGTTCTTGGGAGAAGCAAGAGGACAACGAGAAGCAC
TTGAACTGGATCAGAAACATCTACAACTTCATGACTCCATACGTTTCCAAAAACCCTAGATTGGCATACCTAAAC
TACAGAGACTTGGACATCGGCATCAACGACCCCAAGAACCCTAACAACTACACTCAGGCTAGAATCTGGGGTGAA
AAGTACTTCGGTAAGAACTTCGACAGATTGGTTAAGGTTAAGACTTTGGTTGACCCTAACAATTTCTTCAGGAAC
GAGCAGTCCATCCCTCCATTGCCAAGACATAGACATTAA
SEQ ID NO: 22
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATCTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAAGAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTCTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTAAGGTTGACCCTAACAAT
TTCTTCAGGAACGAGCAGTCCATCCCTCCATTGCCAAGACATAGACATTAA
SEQ ID NO: 23
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTTCTGTTTTGGACAGACAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGCAACCAATCCCAGAGTCCGTTTTCGTTAAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 24
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACCCTACACCCAATACTGCTATC
CACACTTACTTCTCTTCCGTTTTCTTGGGTGGTGTTGACTCCTTGGTTGATTTGATGAACAAGTCCTTCCCAGAG
TTGGGCATCAAGAAAACTGACTGTAGACAGTTGTCCTGGATCGACACTATCATCTTCTACTCCGGTGTTGTAAAC
TACGACACAGACAACTTCAACAAAGAGATCTTGTTGGACAGATCCGCTGGCCAGAACGGTGCTTTCAAGATCAAG
TTGGACTACGTTAAGAAGCCAATCCCAGAGTCCGTTTTCGTTCAGATTTTGGAGAAGTTGTACGAAGAGGACATC
GGTGCTGGAATGTACGCTTTGTACCCTTACGGTGGCATCATGGACGAAATCTCCGAGTCCGCTATTCCCTTCCCA
CACCGTGCTGGTATCTTGTACGAGTTGTGGTACATCTGTTCTTGGGAGAAGCAAGAGGACAACGAGAAGCACTTG
AACTGGATCAGAAACATCTACAACTTCATGACTCCATACGTTTCCAAAAACCCTAGATTGGCATACCTAAACTAC
AGAGACTTGGACATCGGCATCAACGACCCCAAGAACCCTAACAACTACACTCAGGCTAGAATCTGGGGTGAAAAG
TACTTCGGTAAGAACTTCGACAGATTGGTTAAGGTTAAGACTTTGGTTGACCCTAACAATTTCTTCAGGAACGAG
CAGTCCATCCCTCCATTGCCAAGACATAGACATTAA
SEQ ID NO: 25
AACGCAAGAGAAGACTTCTTGAAGTGTTTTTCCCAGCACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAAGTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAAACGGAGCTATCCACGAATTGGTTAAGTTGGTTAACAAGTGGCAGAACATT
GCTTACAAGTACGACGAAGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTAAGATTTTGGAGAAGTTG
TACGAAGAGGACATCGGTGCTGGAATGTACGCTTTGTACCCTTACGGTGGCATCATGGACGAAAAGTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 26
AACGCAAGAGAAAACTTCTTGAAGTGTTTTTCCCAGCACATCCCCAACAACGCTACAAACTTGAAGTTGGTATAC
ACTCAGAATAATCCCTTGTACCAATCCGTTTTGAACTCCACAATCCACAACTTGAGATTCACATCCGACACTACT
CCAAAGCCCTTGGTTATCGTTACTCCCTCCCACGTTTCCCACATCCAGGCTACTATTTTGTGTTCCAAGAAGGTT
GGATTGCAGATCAGAACAAGATCCGGTGGTCACGACTCTGAAGGTTTGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAAATGTCGAAATCCACGCTTTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTACAGTTTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 27
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGGACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 28
AACGCAAGAGAAAACTTCTTGAAGTGTTTTTCCCAGTACATCCCCAACGACGCTACAAACTTGAAGTTGGTATAC
ACTCAGAATAATCCCTTGTACATGTCCGTTTTGGACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 29
AACGCAAGAGAAAACTTCTTGAAGTGTTTTTCCCAGTACATCCCCAACGACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGCAA
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 30
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGGACTCCACAATCCACAACTTGAGATTCACATCCGACACTACT
CCAAAGCCCTTGGTTATCGTTACTCCCTCCCACGTTTCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAAGCGGTGCT
ACATTGGGTGAAGTTTATTACTGGGTTAACGAGAAGAACGAGAACCTCTCCTTGGCCGCTGGTTACTGTCCAACC
GTTGGTGCTGGTGGTCATTTCTCTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGGTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTAAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 31
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCTGTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACGACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 32
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAAAACTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 33
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGATTCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 34
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGATAACTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 35
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGGACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 36
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATCTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAAGAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTCTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTAAGGTTGACCCTAACAAT
TTCTTCAGGAACGAGCAGTCCATCCCTCCATTGCCAAGACATAGACATTAA
SEQ ID NO: 37
AACGCAAGAGAAAACTTCTTGAAGTGTTTTTCCCAGCACATCCCCAACGATGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTTCCTGGTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 38
AACGCAAGAGAAAACTTCTTGAAGTGTTTTTCCCAGCACATCCCCAACAACGCTACAAACTTGAAGTTGGTATAC
ACTCAGAATAATCCCTTGTACATGTCCGTTTTGGACTCCACAATCCACAACTTGAGATTCACATCCGACACTACT
CCAAAGCCCTTGGTTATCGTTACTCCCTCCAACGTTGCCCACATCCAGGCTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATCTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAAGAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTCTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTAAGGTTGACCCTAACAAT
TTCTTCAGGAACGAGCAGTCCATCCCTCCATTGCCAAGACATAGACATTAA
SEQ ID NO: 39
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACGCTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 40
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACACTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 41
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACCCTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 42
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTCTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAACGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 43
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCAGAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAAAGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 44
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCAGAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAGAGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 45
AACGCAAGAGAAAACTTCTTGAAGTGTTTTTCCCAGTACATCCCCAACAACGCTACAAACTTGAAGTTGGTATAC
ACTCAGAATAATCCCTTGTACATGTCCGTTTTGAACTCCACAATCCACAACTTGAGATTCACATCCGACACTACT
CCAAAGCCCTTGGTTATCGTTACTCCCTCCCACGTTTCCCACATCCAGGGTACTATTTTGTGTTCCAAGAAGGTT
GGATTGCAGATCAGAACAAGATCCGGTGGTCACGACTCTGAAGGTATGTCCTACATTTCCCAGGTTCCCTTCGTT
ATCGTTGACCTTAGAAACATGCGTTCCATCAAGATCGACGTTCACTCCCAGACTGCTTGGGTTGAAGCAGGTGCT
ACGTTGGGTGAAGTTTATTACTGGGTTAACGAGAAGAACGAGAACCTCTCCTTGGCCGCTGGTTACTGTCCAACC
GTTGGTGCTGGTGGTCATTTCGGTGGAGGTGGTTATGGTCCATTGATGAGAAACTACGGACTTGCTGCTGACAAC
ATCATCGACGCTCACTTGGTTAACGTTCACGGTAAGGTTTTGGACAGAAAGTCCATGGGAGAGGACTTGTTCTGG
GCTTTGCGTGGTGGTGGTGCTGAATCCTTCGGTATTATCGTTGCTTGGAAGATCAGACTTGTTGCTGTTCCCAAG
TCCACTATGTTCTCCGTTAAGAAAATCATGGAAATCCACGAATTGGTTAAGTTGGTTAACAAGTGGCAGAACATT
GCTTACAAGTACGACAAGGACTTGTTGTTGATGACTCACTTCATCACTAGAAACATCACTGACAACCAGGGTAAG
AACAAAACTGCTATCCACACTTACTTCTCTTCCGTTTTCTTGGGTGGTGTTGACTCCTTGGTTGATTTGATGAAC
AAGTCCTTCCCAGAGTTGGGCATCAAGAAAACTGACTGTAGACAGTTGTCCTGGATCGACACTATCATCTTCTAC
TCCGGTGTTGTAAACTACGACACAGACAACTTCAACAAAGAGATCTTGTTGGACAGATCCGCTGGCCAGAGAGGT
GCTTTCAAGATCAAGTTGGACTACGTTAAGAAGCCAATCCCAGAGTCCGTTTTCGTTCAGATTTTGGAGAAGTTG
TACGAAGAGGACATCGGTGCTGGAATGTACGCTTTGTACCCTTACGGTGGCATCATGGACGAAATCTCCGAGTCC
GCTATTCCCTTCCCACACCGTGCTGGTATCTTGTACGAGTTGTGGTACATCGCTTCTTGGGAGAAGCAAGAGGAC
AACGAGAAGCACTTGAACTGGATCAGAAACATCTACAACTTCATGACTCCATACGTTTCCAAAAACCCTAGATTG
GCATACCTAAACTACAGAGACTTGGACATCGGCATCAACGACCCCAAGAACCCTAACAACTACACTCAGGCTAGA
ATCTGGGGTGAAAAGTACTTCGGTAAGAACTTCGACAGATTGGTTAAGGTTAAGACTTTGGTTGACCCTAACAAT
TTCTTCAGGAACGAGCAGTCCATCCCTCCATTGCCAAGACATAGACATTAA
SEQ ID NO: 46
ATGAGATTTCCTTCAATTTTTACTGCAGTTTTATTCGCAGCATCCTCCGCATTAGCTGCTCCAGTCAACACTACA
ACAGAAGATGAAACGGCACAAATTCCGGCTGAAGCTGTCATCGGTTACTCAGATTTAGAAGGGGATTTCGATGTT
GCTGTTTTGCCATTTTCCAACAGCACAAATAACGGGTTATTGTTTATAAATACTACTATTGCCAGCATTGCTGCT
AAAGAAGAAGGGGTATCTCTCGAGAAAAGAGAGGCTGAAGCT
SEQ ID NO: 47
ATGCAATACAAAAAGACTTTGGTTGCCTCTGCTTTGGCCGCTACTACATTGGCCGCCTATGCTCCATCTGAGCCT
TGGTCCACTTTGACTCCAACAGCCACTTACAGCGGTGGTGTTACCGACTACGCTTCCACCTTCGGTATTGCCGTT
CAACCAATCTCCACTACATCCAGCGCATCATCTGCAGCCACCACAGCTAGCTTGGAGAAGAGGGAGGCTGATGCT
SEQ ID NO: 48
ATGAGATTCCCATCCATCTTCACTGCTGTTTTGTTCGCTGCTTCTTCCGCTTTGGCTGCTAGAGCCTATGCTCCA
TCTGAGCCTTGGTCCACTTTGACTCCAACAGCCACTTACAGCGGTGGTGTTACCGACTACGCTTCCACCTTCGGT
ATTGCCGTTCAACCAATCTCCACTACATCCAGCGCATCATCTGCAGCCACCACAGCCTCATCTgCTAGCTTGGAG
AAGAGGGAGGCTGATGCT
SEQ ID NO: 49
ATGAGATTCCCATCCATCTTCACTGCTGTTTTGTTCGCTGCTTCTTCCGCTTTGGCTGCTCCAGTTAACACTACT
ACTGAGGACGAGACTGCTCAGATTCCAGCTGAAGCTGTTATTGGTTACTCCGACTTGGAAGGTGACTTCGACGTT
GCTGTTTTGCCATTCTCCAACTCCACTAACAACGGTTTGTTGTTCATCAACACTACAATCGCTTCCATTGCTGCT
AAAGAAGAGGGAGTTTCCTTGGAGAAGAGAGAGGCTGAAGCTCGCTGCTTCTTCCGCTTTGGCTGCTCCAGTTAA
CACTACTACTGAGGACGAGACTGCTCAGATTCCAGCTGAAGCTGTTATTGGTTACTCCGACTTGGAAGGTGACTT
CGACGTTGCTGTTTTGCCATTCTCCAACTCCACTAACAACGGTTTGTTGTTCATCAACACTACAATCGCTTCCAT
TGCTGCTAAAGAAGAGGGAGTTTCCTTGGAGAAGAGAGAGGCTGAAGC
SEQ ID NO: 50
ATGCTTTTGCAAGCTTTCCTTTTCCTTTTGGCTGGTTTTGCAGCCAAAATATCTGCAGGATCCGCTAGCTTGGAG
AAGAGGGAGGCTGAAGCT
SEQ ID NO: 51
ATGTTTTCTCCTATTCTAAGTCTGGAAATTATTCTCGCTTTGGCTACTCTCCAATCAGTCTTTGCGGCTAGCTTG
GAGAAGAGGGAGGCTGAAGCT
SEQ ID NO: 52
TGTACAGGAACTTAATAATTGCTACTGCCCTTACTTGCGGTGCATACAGTGCCTACGTGCCTTCCGAACCATGGA
GCACACTGACACCTGATGCTAGCCTTGAAAGTGCCCTCAAAGATTACTCACAAACTTTTGGAATAGCTATTAAGT
CCTTAGATGCCGACGGATCCGCTAGCTTGGAGAAGAGGGAGGCTGAAGCT
SEQ ID NO: 53
ATGAGGCAGGTTTGGTTCTCTTGGATTGTGGGATTGTTCCTATGTTTTTTCAACGTGTCTTCTGCTGCTAGCTTG
GAGAAGAGGGAGGCTGATGCT
SEQ ID NO: 54
ATGAGGCAGGTTTGGTTCTCTTGGATTGTGGGATTGTTCCTATGTTTTTTCAACGTGTCTTCTGCTGCTCCAGTT
AACACTACTACTGAGGACGAGACTGCTCAGATTCCAGCTGAAGCTGTTATTGGTTACTCCGACTTGGAAGGTGAC
TTCGACGTTGCTGTTTTGCCATTCTCCAACTCCACTAACAACGGTTTGTTGTTCATCAACACTACAATCGCTTCC
ATTGCTGCTAAAGAAGAGGGAGCTAGCTTGGAGAAGAGGGAGGCTGATGCT
SEQ ID NO: 55
ATGTTCAACCTGAAAACTATTCTCATCTCAACACTTGCATCGATCGCTGTTGCCGCTAGCTTGGAGAAGAGGGAG
GCTGATGCT
SEQ ID NO: 56
TACCCATACGATGTTCCAGATTACGCT
SEQ ID NO: 57
CATCACCATCACCATCAC
SEQ ID NO: 58
CATAACCATAATCACAATCACAAC
SEQ ID NO: 59
GATCTAACATCCAAAGACGAAAGGTTGAATGAAACCTTTTTGCCATCCGACATCCACAGGTCCATTCTCACACAT
AAGTGCCAAACGCAACAGGAGGGGATACACTAGCAGCAGACCGTTGCAAACGCAGGACCTCCACTCCTCTTCTCC
TCAACACCCACTTTTGCCATCGAAAAACCAGCCCAGTTATTGGGCTTGATTGGAGCTCGCTCATTCCAATTCCTT
CTATTAGGCTACTAACACCATGACTTTATTAGCCTGTCTATCCTGGCCCCCCTGGCGAGGTTCATGTTTGTTTAT
TTCCGAATGCAACAAGCTCCGCATTACACCCGAACATCACTCCAGATGAGGGCTTTCTGAGTGTGGGGTCAAATA
GTTTCATGTTCCCCAAATGGCCCAAAACTGACAGTTTAAACGCTGTCTTGGAACCTAATATGACAAAAGCGTGAT
CTCATCCAAGATGAACTAAGTTTGGTTCGTTGAAATGCTAACGGCCAGTTGGTCAAAAAGAAACTTCCAAAAGTC
GGCATACCGTTTGTCTTGTTTGGTATTGATTGACGAATGCTCAAAAATAATCTCATTAATGCTTAGCGCAGTCTC
TCTATCGCTTCTGAACCCCGGTGCACCTGTGCCGAAACGCAAATGGGGAAACACCCGCTTTTTGGATGATTATGC
ATTGTCTCCACATTGTATGCTTCCAAGATTCTGGTGGGAATACTGCTGATAGCCTAACGTTCATGATCAAAATTT
AACTGTTCTAACCCCTACTTGACAGCAATATATAAACAGAAGGAAGCTGCCCTGTCTTAAACCTTTTTTTTTATC
ATCATTATTAGCTTACTTTCATAATTGCGACTGGTTCCAATTGACAAGCTTTTGATTTTAACGACTTTTAACGAC
AACTTGAGAAGATCAAAAAACAACTAATTATTCGAAACG
SEQ ID NO: 60
TTATTTTTTGACCGAATTCTTTTTTTCAGACCATATGACCGGTCCATCTTCTACGGGGGGATTATCTATGCTTTG
ACCTCTATCTTGATTCTTTTATGATTCAAATCACTTTTACGTTATTTATTACTTACTGGTTATTTACTTAGCGCC
TTTTCTGAAAAACATTTACTAAAAATCATACATCGGCACTCTCAAACACGACAGATTGTGATCAAGAAGCAGAGA
CAATCACCACTAAGGTTGCACATTTGAGCCAGTAGGCTCCTAATAGAGGTTCGATACTTATTTTGATAATACGAC
ATATTGTCTTACCTCTGAATGTGTCAATACTCTCTCGTTCTTCGTCTCGTCAGCTAAAAATATAACACTTCGAGT
AAGATACGCCCAATTGAAGGCTACGAGATACCAGACTATCACTAGTAGAACTTTGACATCTGCTAAAGCAGATCA
AATATCCATTTATCCAGAATCAATTACCTTCCTTTAGCTTGTCGAAGGCATGAAAAAGCTACATGAAAATCCCCA
TCCTTGAAGTTTTGTCAGCTTAAAGGACTCCATTTCCTAAAATTTCAAGCAGTCCTCTCAACTAAATTTTTTTCC
ATTCCTCTGCACCCAGCCCTCTTCATCAACCGTCCAGCCTTCTCAAAAGTCCAATGTAAGTAGCCTGCAAATTCA
GGTTACAACCCCTCAATTTTCCATCCAAGGGCGATCCTTACAAAGTTAATATCGAACAGCAGAGACTAAGCGAGT
CATCATCACCACCCAACGATGGTGAAAAACTTTAAGCATAGATTGATGGAGGGTGTATGGCACTTGGCGGCTGCA
TTAGAGTTTGAAACTATGGGGTAATACATCACATCCGGAACTGATCCGACTCCGAGATCATATGCAAAGCACGTG
ATGTACCCCGTAAACTGCTCGGATTATCGTTGCAATTCATCGTCTTAAACAGTACAAGAAACTTTATTCATGGGT
CATTGGACTCTGATGAGGGGCACATTTCCCCAATGATTTTTTGGGAAAGAAAGCCGTAAGAGGACAGTTAAGCGA
AAGAGACAAGACAACGAACAGCAAAAGTGACAGCTGTCAGCTACCTAGTGGACAGTTGGGAGTTTCCAATTGGTT
GGTTTTGAATTTTTACCCATGTTGAGTTGTCCTTGCTTCTCCTTGCAAACAATGCAAGTTGATAAGACATCACCT
TCCAAGATAGGCTATTTTTGTCGCATAAATTTTTGTCTCGGAGTGAAAACCCCTTTTATGTGAACAGATTACAGA
AGCGTCCTACCCTTCACCGGTTGAGATGGGGAGAAAATTAAGCGATGAGGAGACGATTATTGGTATAAAAGAAGC
AACCAAAATCCCTTATTGTCCTTTTCTGATCAGCATCAAAGAATATTGTCTTAAAACGGGCTTTTAACTACATTG
TTCTTACACATTGCAAACCTCTTCCTTCTATTTCGGATCAACTGTATTGACTACATTGATCTTTTTTAACGAAGT
TTACGACTTACTAAATCCCCACAAACAAATCAACTGAGAAAA
SEQ ID NO: 61
TGAGATAACAGAGTTGGGTAACTAGAGAGAATAATAGACGTATGCATGATTACTACACAACGGATGTCGCACTCT
TTCCTTAGTTAAAACTATCATCCAATCACAAGATGCGGGCTGGAAAGACTTGCTCCCGAAGGATAATCTTCTGCT
TCTATCTCCCTTCCTCATATGGTTTCGCAGGGCTCATGCCCCTTCTTCCTTCGAACTGCCCGATGAGGAAGTCCT
TAGCCTATCAAAGAATTCGGGACCATCATCGATTTTTAGAGCCTTACCTGATCGCAATCAGGATTTCACTACTCA
TATAAATACATCGCTCAAAGCTCCAACTTTGCTTGTTCATACAATTCTTGATATTCAC
SEQ ID NO: 62
ATTCTGGCCCTTGCACCTGATCGCGAAGGTGGAAATGGCAGAAGGATCAGCCTGGACGAAGCAACCAGTTCCAAC
TGCTAAGTAAAGAAGATGCTAGACGAAGGAGACTTCAGAGGTGAAAAGTTTGCAAGAAGAGAGCTGCGGGAAATA
AATTTTCAATTTAAGGACTTGAGTGCGTCCATATTCGTGTACGTGTCCAACTGTTTTCCATTACCTAAGAAAAAC
ATAAAGATTAAAAAGATAAACCCAATCGGGAAACTTTAGCGTGCCGTTTCGGATTCCGAAAAACTTTTGGAGCGC
CAGATGACTATGGAAAGAGGAGTGTACCAAAATGGCAAGTCGGGGGCTACTCACCGGATAGCCAATACATTCTCT
AGGAACCAGGGATGAATCCAGGTTTTTGTTGTCACGGTAGGTCAAGCATTCACTTCTTAGGAATATCTCGTTGAA
AGCTACTTGAAATCCCATTGGGTGCGGAACCAGCTTCTAATTAAATAGTTCGATGATGTTCTCTAAGTGGGACTC
TACGGCTCAAACTTCTACACAGCATCATCTTAGTAGTCCCTTCCCAAAACACCATTCTAGGTTTCGGAACGTAAC
GAAACAATGTTCCTCTCTTCACATTGGGCCGTTACTCTAGCCTTCCGAAGAACCAATAAAAGGGACCGGCTGAAA
CGGGTGTGGAAACTCCTGTCCAGTTTATGGCAAAGGCTACAGAAATCCCAATCTTGTCGGGATGTTGCTCCTCCC
AAACGCCATATTGTACTGCAGTTGGTGCGCATTTTAGGGAAAATTTACCCCAGATGTCCTGATTTTCGAGGGCTA
CCCCCAACTCCCTGTGCTTATACTTAGTCTAATTCTATTCAGTGTGCTGACCTACACGTAATGATGTCGTAACCC
AGTTAAATGGCCGAAAAACTATTTAAGTAAGTTTATTTCTCCTCCAGATGAGACTCTCCTTCTTTTCTCCGCTAG
TTATCAAACTATAAACCTATTTTACCTCAAATACCTCCAACATCACCCACTTAAACA
SEQ ID NO: 63
ATCTTCATTGATGAAACGTTGTGATCGGTGTGACTTTTATAGTAAAAGCTACAACTGTTTGAAATACCAAGATAT
CATTGTGAATGGCTCAAAAGGGTAATACATCTGAAAAACCTGAAGTGTGGAAAATTCCGATGGAGCCAACTCATG
ATAACGCAGAAGTCCCATTTTGCCATCTTCTCTTGGTATGAAACGGTAGAAAATGATCCGAGTATGCCAATTGAT
ACTCTTGATTCATGCCCTATAGTTTGCGTAGGGTTTAATTGATCTCCTGGTCTATCGATCTGGGACGCAATGTAG
ACCCCATTAGTGGAAACACTGAAAGGGATCCAACACTCTAGGCGGACCCGCTCACAGTCATTTCAGGACAATCAC
CACAGGAATCAACTACTTCTCCCAGTCTTCCTTGCGTGAAGCTTCAAGCCTACAACATAACACTTCTTACTTAAT
CTTTGATTCTCGAATTGTTTACCCAATCTTGACAACTTAGCCTAAGCAATACTCTGGGGTTATATATAGCAATTG
CTCTTCCTCGCTGTAGCGTTCATTCCATCTTTCTA
SEQ ID NO: 64
TGTCATCTGCTGATGCTGTGAGGGAGAAAGAAGTAGGGGTGATACATGGTTTATAGGCAAAGCATGTTTGTTTCA
GATCAAAGATTAGCGTTTCAAAGTTGTGGAAAAGTGACCATGCAACAATATGCAACACATTCGGATTATCTGATA
AGTTTCAAAGCTACTAAGTAAGCCCGTTTCAAGTCTCCAGACCGACATCTGCCATCCAGTGATTTTCTTAGTCCT
GAAAAATACGATGTGTAAACATAAACCACAAAGATCGGCCTCCGAGGTTGAACCCTTACGAAAGAGACATCTGGT
AGCGCCAATGCCAAAAAAAAATCACACCAGAAGGACAATTCCCTTCCCCCCCAGCCCATTAAAGCTTACCATTTC
CTATTCCAATACGTTCCATAGAGGGCATCGCTCGGCTCATTTTCGCGTGGGTCATACTAGAGCGGCTAGCTAGTC
GGCTGTTTGAGCTCTCTAATCGAGGGGTAAGGATGTCTAATATGTCATAATGGCTCACTATATAAAGAACCCGCT
TGCTCAACCTTCGACTCCTTTCCCGATCCTTTGCTTGTTGCTTCTTCTTTTATAACAGGAAACAAAGGAATTTAT
ACACTTTAA
SEQ ID NO: 65
AAATTAATCCATAAGATAAGGCAAATGTGCTTAAGTAATTGAAAACAGTGTTGTGATTATATAAGCATGGTATTT
GAATAGAACTACTGGGGTTAACTTATCTAGTAGGATGGAAGTTGAGGGAGATCAAGATGCTTAAAGAAAAGGATT
GGCCAATATGAAAGCCATAATTAGCAATACTTATTTAATCAGATAATTGTGGGGCATTGTGACTTGACTTTTACC
AGGACTTCAAACCTCAACCATTTAAACAGTTATAGAAGACGTACCGTCACTTTTGCTTTTAATGTGATCTAAATG
TGATCACATGAACTCAAACTAAAATGATATCTTTTACTGGACAAAAATGTTATCCTGCAAACAGAAAGCTTTCTT
CTATTCTAAGAAGAACATTTACATTGGTGGGAAACCTGAAAACAGAAAATAAATACTCCCCAGTGACCCTATGAG
CAGGATTTTTGCATCCCTATTGTAGGCCTTTCAAACTCACACCTAATATTTCCCGCCACTCACACTATCAATGAT
CACTTCCCAGTTCTCTTCTTCCCCTATTCGTACCATGCAACCCTTACACGCCTTTTCCATTTCGGTTCGGATGCG
ACTTCCAGTCTGTGGGGTACGTAGCCTATTCTCTTAGCCGGTATTTAAACATACAAATTCACCCAAATTCTACCT
TGATAAGGTAATTGATTAATTTCATAAAT
SEQ ID NO: 66
TCCAAACCAAACGGTCTAGCAAAAACGATAACTTTAAAGAACTTTTCAATTGGTTTTGTACACTACCACCGGTTT
ACTACCTCTGCCTTCGGTTCTTCTCCTCACATTTTTCGCAACTGGGATAGCGTAGCCTAAAGTGTCACATGCTCG
CTGCTCACATTCCCTACACAACAGAGATTGTCAGCAGAGGAAATTGAGCTCCACCATTCAACACTTGTGGATTTA
TGATAGTCTGTGCTATCAGCTCTCTTTTTTTTGTTGCTGTAGAATTTACCGTGCTAGCAACCTTTTAAACTTTGT
TTAGCTCTCCTTCCCTCTTCCATTCATCTGTTTCGGTCCGATCCGTCTCTGGTCATCTCCTCCGCATTTTTTTTT
TACCGTTAGCGATAGGGGTCAGATCAATTCAATCAGTTTTGGCAAGGGTATTTAAAGGTGGCGAAATCCCCCTCC
GTTTGTTGAACACATCCAACTATTCTCAACCCAACCATCTAACTAATCGTA
SEQ ID NO: 67
CGCAGCGTTTTCTGACGGTACTAGAGGACTCTTAGGGGAAGGTAGAATCAATAAAGATCATATTAGGTAAGCAAA
TTTTGGATGGAATAGGAGACTAGGTGTGGATGCGCGATCTCGCCAAATTGCACGACCAGAGTGGATGCCGGATGG
TGGTAAACCGTTTCTTCCTTTTTACCACCCAAGTGCGAGTGAAACACCCCATGGCTGCTCTCCGATTGCCCCTCT
ACAGGCATAAGGGTGTGACTTTGTGGGCTTGAATTTTACACCCCCTCCAACTTTTCTCGCATCAATTGATCCTGT
TACCAATATTGCATGCCCGGAGGAGACTTGCCCCCTAATTTCGCGGCGTCGTCCCGGATCGCAGGGTGAGACTGT
AGAGACCCCACATAGTGACAATGATTATGTAAGAAGAGGGGGGTGATTCGGCCGGCTATCGAACTCTAACAACTA
GGGGGGTGAACAATGCCCAGCAGTCCTCCCCACTCTTTGACAAATCAGTATCACCGATTAACACCCCAAATCTTA
TTCTCAACGGTCCCTCATCCTTGCACCCCTCTTTGGACAAATGGCAGTTAGCATTGGTGCACTGACTGACTGCCC
AACCTTAAACCCAAATTTCTTAGAAGGGGCCCATCTAGTTAGCGAGGGGTGAAAAATTCCTCCATCGGAGATGTA
TTGACCGTAAGTTGCTGCTTAAAAAAAATCAGTTCAGATAGCGAGACTTTTTTGATTTCGCAACGGGAGTGCCTG
TTCCATTCGATTGCAATTCTCACCCCTTCTGCCCAGTCCTGCCAATTGCCCATGAATCTGCTAATTTCGTTGATT
CCCACCCCCCTTTCCAACTCCACAAATTGTCCAATCTCGTTTTCCATTTGGGAGAATCTGCATGTCGACTACATA
AAGCGACCGGTGTCCGAAAAGATCTGTGTAGTTTTCAACATTTTGTGCTCCCCCCGCTGTTTGAAAACGGGGGTG
AGCGCTCTCCGGGGTGCGAATTCGTGCCCAATTCCTTTCACCCTGCCTATTGTAGACGTCAACCCGCATCTGGTG
CGAATATAGCGCACCCCCAATGATCACACCAACAATTGGTCCACCCCTCCCCAATCTCTAATATTCACAATTCAC
CTCACTATAAATACCCCTGTCCTGCTCCCAAATTCTTTTTTCCTTCTTCCATCAGCTACTAGCTTTTATCTTATT
TACTTTACGAAA
SEQ ID NO: 68
TGTGTCTGTAAATTTAAATCCAAAGAAAAATAAAGGGCACTACATAGAACAACACAATCAACCTACATAGCTCTC
TTTTTTTTTTTTTTTTTTTTTTTCTTTTTTGTTTTTTCTCAATTTTCTTCATTTTTTGCATTGTTACTACTCTCC
CATTAAGGGAAATTCTACCAGGAAAACCGAAGAAGACAAATGCAAAAGGGAAATATGAATACGCATGTATGCGCG
CAAAACCGCACTTACAGAGGGCATTAGGACATT
SEQ ID NO: 69
AGATCTAACATCCAAAGACGAAAGGTTGAATGAAACCTTTTTGCCATCCGACATCCACAGGTCCATTCTCACACA
TAAGTGCCAAACGCAACAGGAGGGGATACACTAGCAGCAGACCGTTGCAAACGCAGGACCTCCACTCCTCTTCTC
CTCAACACCCACTTTTGCCATCGAAAAACCAGCCCAGTTATTGGGCTTGATTGGAGCTCGCTCATTCCAATTCCT
TCTATTAGGCTACTAACACCATGACTTTATTAGCCTGTCTATCCTGGCCCCCCTGGCGAGGTTCATGTTTGTTTA
TTTCCGAATGCAACAAGCTCCGCATTACACCCGAACATCACTCCAGATGAGGGCTTTCTGAGTGTGGGGTCAAAT
AGTTTCATGTTCCCCAAATGGCCCAAAACTGACAGTTTAAACGCTGTCTTGGAACCTAATATGACAAAAGCGTGA
TCTCATCCAAGATGAACTAAGTTTGGTTCGTTGAAATGCTAACGGCCAGTTGGTCAAAAAGAAACTTCCAAAAGT
CGGCATACCGTTTGTCTTGTTTGGTATTGATTGACGAATGCTCAAAAATAATCTCATTAATGCTTAGCGCAGTCT
CTCTATCGCTTCTGAACCCCGGTGCACCTGTGCCGAAACGCAAATGGGGAAACACCCGCTTTTTGGATGATTATG
CATTGTCTCCACATTGTATGCTTCCAAGATTCTGGTGGGAATACTGCTGATAGCCTAACGTTCATGATCAAAATT
TAACTGTTCTAACCCCTACTTGACAGCAATATATAAACAGAAGGAAGCTGCCCTGTCTTAAACCTTTTTTTTTAT
CATCATTATTAGCTTACTTTCATAATTGCGACTGGTTCCAATTGACAAGCTTTTGATTTTAACGACTTTTAACGA
CAACTTGAGAAGATCAAAAAACAACTAATTATTCGAAACGGAATTCGCTGGATCCTACGTAGAATTCCCTAGGGC
GGCCGCGAATTAATTCGCCTTAGACATGACTGTTCCTCAGTTCAAGTTGGGCACTTACGAGAAGACCGGTCTTGC
TAGATTCTAATCAAGAGGATGTCAGAATGCCATTTGCCTGAGAGATGCAGGCTTCATTTTTGATACTTTTTTATT
TGTAACCTATATAGTATAGGATTTTTTTTGTCATTTTGTTTCTTCTCGTACGAGCTTGCTCCTGATCAGCCTATC
TCGCAGCTGATGAATATCTTGTGGTAGGGGTTTGGGAAAATCATTCGAGTTTGATGTTTTTCTTGGTATTTCCCA
CTCCTCTTCAGAGTACAGAAGATTAAGTGAGAAGTTCGTTTGTGCAAGCTTATCGATAAGCTTTAATGCGGTAGT
TTATCACAGTTAAATTGCTAACGCAGTCAGGCACCGTGTATGAAATCTAACAATGCGCTCATCGTCATCCTCGGC
ACCGTCACCCTGGATGCTGTAGGCATAGGCTTGGTTATGCCGGTACTGCCGGGCCTCTTGCGGGATATCGTCCAT
TCCGACAGCATCGCCAGTCACTATGGCGTGCTGCTAGCGCTATATGCGTTGATGCAATTTCTATGCGCACCCGTT
CTCGGAGCACTGTCCGACCGCTTTGGCCGCCGCCCAGTCCTGCTCGCTTCGCTACTTGGAGCCACTATCGACTAC
GCGATCATGGCGACCACACCCGTCCTGTGGATCTATCGAATCTAAATGTAAGTTAAAATCTCTAAATAATTAAAT
AAGTCCCAGTTTCTCCATACGAACCTTAACAGCATTGCGGTGAGCATCTAGACCTTCAACAGCAGCCAGATCCAT
CACTGCTTGGCCAATATGTTTCAGTCCCTCAGGAGTTACGTCTTGTGAAGTGATGAACTTCTGGAAGGTTGCAGT
GTTAACTCCGCTGTATTGACGGGCATATCCGTACGTTGGCAAAGTGTGGTTGGTACCGGAGGAGTAATCTCCACA
ACTCTCTGGAGAGTAGGCACCAACAAACACAGATCCAGCGTGTTGTACTTGATCAACATAAGAAGAAGCATTCTC
GATTTGCAGGATCAAGTGTTCAGGAGCGTACTGATTGGACATTTCCAAAGCCTGCTCGTAGGTTGCAACCGATAG
GGTTGTAGAGTGTGCAATACACTTGCGTACAATTTCAACCCTTGGCAACTGCACAGCTTGGTTGTGAACAGCATC
TTCAATTCTGGCAAGCTCCTTGTCTGTCATATCGACAGCCAACAGAATCACCTGGGAATCAATACCATGTTCAGC
TTGAGACAGAAGGTCTGAGGCAACGAAATCTGGATCAGCGTATTTATCAGCAATAACTAGAACTTCAGAAGGCCC
AGCAGGCATGTCAATACTACACAGGGCTGATGTGTCATTTTGAACCATCATCTTGGCAGCAGTAACGAACTGGTT
TCCTGGACCAAATATTTTGTCACACTTAGGAACAGTTTCTGTTCCGTAAGCCATAGCAGCTACTGCCTGGGCGCC
TCCTGCTAGCACGATACACTTAGCACCAACCTTGTGGGCAACGTAGATGACTTCTGGGGTAAGGGTACCATCCTT
CTTAGGTGGAGATGCAAAAACAATTTCTTTGCAACCAGCAACTTTGGCAGGAACACCCAGCATCAGGGAAGTGGA
AGGCAGAATTGCGGTTCCACCAGGAATATAGAGGCCAACTTTCTCAATAGGTCTTGCAAAACGAGAGCAGACTAC
ACCAGGGCAAGTCTCAACTTGCAACGTCTCCGTTAGTTGAGCTTCATGGAATTTCCTGACGTTATCTATAGAGAG
ATCAATGGCTCTCTTAACGTTATCTGGCAATTGCATAAGTTCCTCTGGGAAAGGAGCTTCTAACACAGGTGTCTT
CAAAGCGACTCCATCAAACTTGGCAGTTAGTTCTAAAAGGGCTTTGTCACCATTTTGACGAACATTGTCGACAAT
TGGTTTGACTAATTCCATAATCTGTTCCGTTTTCTGGATAGGACGACGAAGGGCATCTTCAATTTCTTGTGAGGA
GGCCTTAGAAACGTCAATTTTGCACAATTCAATACGACCTTCAGAAGGGACTTCTTTAGGTTTGGATTCTTCTTT
AGGTTGTTCCTTGGTGTATCCTGGCTTGGCATCTCCTTTCCTTCTAGTGACCTTTAGGGACTTCATATCCAGGTT
TCTCTCCACCTCGTCCAACGTCACACCGTACTTGGCACATCTAACTAATGCAAAATAAAATAAGTCAGCACATTC
CCAGGCTATATCTTCCTTGGATTTAGCTTCTGCAAGTTCATCAGCTTCCTCCCTAATTTTAGCGTTCAACAAAAC
TTCGTCGTCAAATAACCGTTTGGTATAAGAACCTTCTGGAGCATTGCTCTTACGATCCCACAAGGTGGCTTCCAT
GGCTCTAAGACCCTTTGATTGGCCAAAACAGGAAGTGCGTTCCAAGTGACAGAAACCAACACCTGTTTGTTCAAC
CACAAATTTCAAGCAGTCTCCATCACAATCCAATTCGATACCCAGCAACTTTTGAGTTGCTCCAGATGTAGCACC
TTTATACCACAAACCGTGACGACGAGATTGGTAGACTCCAGTTTGTGTCCTTATAGCCTCCGGAATAGACTTTTT
GGACGAGTACACCAGGCCCAACGAGTAATTAGAAGAGTCAGCCACCAAAGTAGTGAATAGACCATCGGGGCGGTC
AGTAGTCAAAGACGCCAACAAAATTTCACTGACAGGGAACTTTTTGACATCTTCAGAAAGTTCGTATTCAGTAGT
CAATTGCCGAGCATCAATAATGGGGATTATACCAGAAGCAACAGTGGAAGTCACATCTACCAACTTTGCGGTCTC
AGAAAAAGCATAAACAGTTCTACTACCGCCATTAGTGAAACTTTTCAAATCGCCCAGTGGAGAAGAAAAAGGCAC
AGCGATACTAGCATTAGCGGGCAAGGATGCAACTTTATCAACCAGGGTCCTATAGATAACCCTAGCGCCTGGGAT
CATCCTTTGGACAACTCTTTCTGCCAAATCTAGGTCCAAAATCACTTCATTGATACCATTATTGTACAACTTGAG
CAAGTTGTCGATCAGCTCCTCAAATTGGTCCTCTGTAACGGATGACTCAACTTGCACATTAACTTGAAGCTCAGT
CGATTGAGTGAACTTGATCAGGTTGTGCAGCTGGTCAGCAGCATAGGGAAACACGGCTTTTCCTACCAAACTCAA
GGAATTATCAAACTCTGCAACACTTGCGTATGCAGGTAGCAAGGGAAATGTCATACTTGAAGTCGGACAGTGAGT
GTAGTCTTGAGAAATTCTGAAGCCGTATTTTTATTATCAGTGAGTCAGTCATCAGGAGATCCTCTACGCCGGACG
CATCGTGGCCGACCTGCAGGGGGGGGGGGGGCGCTGAGGTCTGCCTCGTGAAGAAGGTGTTGCTGACTCATACCA
GGCCTGAATCGCCCCATCATCCAGCCAGAAAGTGAGGGAGCCACGGTTGATGAGAGCTTTGTTGTAGGTGGACCA
GTTGGTGATTTTGAACTTTTGCTTTGCCACGGAACGGTCTGCGTTGTCGGGAAGATGCGTGATCTGATCCTTCAA
CTCAGCAAAAGTTCGATTTATTCAACAAAGCCGCCGTCCCGTCAAGTCAGCGTAATGCTCTGCCAGTGTTACAAC
CAATTAACCAATTCTGATTAGAAAAACTCATCGAGCATCAAATGAAACTGCAATTTATTCATATCAGGATTATCA
ATACCATATTTTTGAAAAAGCCGTTTCTGTAATGAAGGAGAAAACTCACCGAGGCAGTTCCATAGGATGGCAAGA
TCCTGGTATCGGTCTGCGATTCCGACTCGTCCAACATCAATACAACCTATTAATTTCCCCTCGTCAAAAATAAGG
TTATCAAGTGAGAAATCACCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAACCGTTATTCATTCGTGAT
TGCGCCTGAGCGAGACGAAATACGCGATCGCTGTTAAAAGGACAATTACAAACAGGAATCGAATGCAACCGGCGC
AGGAACACTGCCAGCGCATCAACAATATTTTCACCTGAATCAGGATATTCTTCTAATACCTGGAATGCTGTTTTC
CCGGGGATCGCAGTGGTGAGTAACCATGCATCATCAGGAGTACGGATAAAATGCTTGATGGTCGGAAGAGGCATA
AATTCCGTCAGCCAGTTTAGTCTGACCATCTCATCTGTAACATCATTGGCAACGCTACCTTTGCCATGTTTCAGA
AACAACTCTGGCGCATCGGGCTTCCCATACAATCGATAGATTGTCGCACCTGATTGCCCGACATTATCGCGAGCC
CATTTATACCCATATAAATCAGCATCCATGTTGGAATTTAATCGCGGCCTCGAGCAAGACGTTTCCCGTTGAATA
TGGCTCATAACACCCCTTGTATTACTGTTTATGTAAGCAGACAGTTTTATTGTTCATGATGATATATTTTTATCT
TGTGCAATGTAACATCAGAGATTTTGAGACACAACGTGGCTTTCCCCCCCCCCCCTGCAGGTCGGCATCACCGGC
GCCACAGGTGCGGTTGCTGGCGCCTATATCGCCGACATCACCGATGGGGAAGATCGGGCTCGCCACTTCGGGCTC
ATGAGCGCTTGTTTCGGCGTGGGTATGGTGGCAGGCCCCGTGGCCGGGGGACTGTTGGGCGCCATCTCCTTGCAT
GCACCATTCCTTGCGGCGGCGGTGCTCAACGGCCTCAACCTACTACTGGGCTGCTTCCTAATGCAGGAGTCGCAT
AAGGGAGAGCGTCGAGTATCTATGATTGGAAGTATGGGAATGGTGATACCCGCATTCTTCAGTGTCTTGAGGTCT
CCTATCAGATTATGCCCAACTAAAGCAACCGGAGGAGGAGATTTCATGGTAAATTTCTCTGACTTTTGGTCATCA
GTAGACTCGAACTGTGAGACTATCTCGGTTATGACAGCAGAAATGTCCTTCTTGGAGACAGTAAATGAAGTCCCA
CCAATAAAGAAATCCTTGTTATCAGGAACAAACTTCTTGTTTCGAACTTTTTCGGTGCCTTGAACTATAAAATGT
AGAGTGGATATGTCGGGTAGGAATGGAGCGGGCAAATGCTTACCTTCTGGACCTTCAAGAGGTATGTAGGGTTTG
TAGATACTGATGCCAACTTCAGTGACAACGTTGCTATTTCGTTCAAACCATTCCGAATCCAGAGAAATCAAAGTT
GTTTGTCTACTATTGATCCAAGCCAGTGCGGTCTTGAAACTGACAATAGTGTGCTCGTGTTTTGAGGTCATCTTT
GTATGAATAAATCTAGTCTTTGATCTAAATAATCTTGACGAGCCAAGGCGATAAATACCCAAATCTAAAACTCTT
TTAAAACGTTAAAAGGACAAGTATGTCTGCCTGTATTAAACCCCAAATCAGCTCGTAGTCTGATCCTCATCAACT
TGAGGGGCACTATCTTGTTTTAGAGAAATTTGCGGAGATGCGATATCGAGAAAAAGGTACGCTGATTTTAAACGT
GAAATTTATCTCAAGATCTCTGCCTCGCGCGTTTCGGTGATGACGGTGAAAACCTCTGACACATGCAGCTCCCGG
AGACGGTCACAGCTTGTCTGTAAGCGGATGCCGGGAGCAGACAAGCCCGTCAGGGCGCGTCAGCGGGTGTTGGCG
GGTGTCGGGGCGCAGCCATGACCCAGTCACGTAGCGATAGCGGAGTGTATACTGGCTTAACTATGCGGCATCAGA
GCAGATTGTACTGAGAGTGCACCATATGCGGTGTGAAATACCGCACAGATGCGTAAGGAGAAAATACCGCATCAG
GCGCTCTTCCGCTTCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGGGAAGCGTGGCGCT
TTCTCAATGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTGCTACAGAGTTCTTGAAGTG
GTGGCCTAACTACGGCTACACTAGAAGGACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTAATTGTTG
CCGGGAAGCTAGAGTAAGTAGTTCGCCAGTTAATAGTTTGCGCAACGTTGTTGCCATTGCTGCAGGCATCGTGGT
GTCACGCTCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGTTATCACT
CATGGTTATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAACACGGGATAATAC
CGCGCCACATAGCAGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGGCGACACGGAAATGTTGAAT
ACTCATACTCTTCCTTTTTCAATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGTGCCACCTGACGTCTAAGAAAC
CATTATTATCATGACATTAACCTATAAAAATAGGCGTATCACGAGGCCCTTTCGTCTTCAAGAATTAATTCTCAT
GTTTGACAGCTTATCATCGATAAGCTGACTCATGTTGGTATTGTGAAATAGACGCAGATCGGGAACACTGAAAAA
TAACAGTTATTATTCG
SEQ ID NO: 70
AGATCTAACATCCAAAGACGAAAGGTTGAATGAAACCTTTTTGCCATCCGACATCCACAGGTCCATTCTCACACA
TAAGTGCCAAACGCAACAGGAGGGGATACACTAGCAGCAGACCGTTGCAAACGCAGGACCTCCACTCCTCTTCTC
CTCAACACCCACTTTTGCCATCGAAAAACCAGCCCAGTTATTGGGCTTGATTGGAGCTCGCTCATTCCAATTCCT
TCTATTAGGCTACTAACACCATGACTTTATTAGCCTGTCTATCCTGGCCCCCCTGGCGAGGTTCATGTTTGTTTA
TTTCCGAATGCAACAAGCTCCGCATTACACCCGAACATCACTCCAGATGAGGGCTTTCTGAGTGTGGGGTCAAAT
AGTTTCATGTTCCCCAAATGGCCCAAAACTGACAGTTTAAACGCTGTCTTGGAACCTAATATGACAAAAGCGTGA
TCTCATCCAAGATGAACTAAGTTTGGTTCGTTGAAATGCTAACGGCCAGTTGGTCAAAAAGAAACTTCCAAAAGT
CGGCATACCGTTTGTCTTGTTTGGTATTGATTGACGAATGCTCAAAAATAATCTCATTAATGCTTAGCGCAGTCT
CTCTATCGCTTCTGAACCCCGGTGCACCTGTGCCGAAACGCAAATGGGGAAACACCCGCTTTTTGGATGATTATG
CATTGTCTCCACATTGTATGCTTCCAAGATTCTGGTGGGAATACTGCTGATAGCCTAACGTTCATGATCAAAATT
TAACTGTTCTAACCCCTACTTGACAGCAATATATAAACAGAAGGAAGCTGCCCTGTCTTAAACCTTTTTTTTTAT
CATCATTATTAGCTTACTTTCATAATTGCGACTGGTTCCAATTGACAAGCTTTTGATTTTAACGACTTTTAACGA
CAACTTGAGAAGATCAAAAAACAACTAATTATTCGAAGgatctgaattcataGCTAGCTTGGAGAAGAGGcCtta
attaactgGGCCGCGAATTAATTCGCCTTAGACATGACTGTTCCTCAGTTCAAGTTGGGCACTTACGAGAAGACC
GGTCTTGCTAGATTCTAATCAAGAGGATGTCAGAATGCCATTTGCCTGAGAGATGCAGGCTTCATTTTTGATACT
TTTTTATTTGTAACCTATATAGTATAGGATTTTTTTTGTCATTTTGTTTCTTCTCGTACGAGCTTGCTCCTGATC
AGCCTATCTCGCAGCTGATGAATATCTTGTGGTAGGGGTTTGGGAAAATCATTCGAGTTTGATGTTTTTCTTGGT
ATTTCCCACTCCTCTTCAGAGTACAGAAGATTAAGTGAGACGTTCGTTTGTGCAAGCTTATCGATAAGCTTTAAT
GCGGTAGTTTATCACAGTTAAATTGCTAACGCAGTCAGGCACCGTGTATGAAATCTAACAATGCGCTCATCGTCA
TCCTCGGCACCGTCACCCTGGATGCTGTAGGCATAGGCTTGGTTATGCCGGTACTGCCGGGCCTCTTGCGGGATA
TCGTCCATTCCGACAGCATCGCCAGTCACTATGGCGTGCTGCTAGGTACAACTTGAGCAAGTTGTCGATCAGCTC
CTCAAATTGGTCCTCTGTAACGGATGACTCAACTTGCACATTAACTTGAAGCTCAGTCGATTGAGTGAACTTGAT
CAGGTTGTGCAGCTGGTCAGCAGCATAGGGAAACACGGCTTTTCCTACCAAACTCAAGGAATTATCAAACTCTGC
AACACTTGCGTATGCAGGTAGCAAGGGAAATGTCATACTTGAAGTCGGACAGTGAGTGTAGTCTTGAGAAATTCT
GAAGCCGTATTTTTATTATCAGTGAGTCAGTCATCAGGAGATCCTCTACGCCGGACGCATCGTGGCCGACCTGCA
GGGGGGGGGGGGGCGCTGAGGTCTGCCTCGTGAAGAAGGTGTTGCTGACTCATACCAGCTGAATCGCCCCATCAT
CCAGCCAGAAAGTGAGGGAGCCACGGTTGATGAGAGCTTTGTTGTAGGTGGACCAGTTGGTGATTTTGAACTTTT
GCTTTGCCACGGAACGGTCTGCGTTGTCGGGAAGATGCGTGATCTGATCCTTCAACTCAGCAAAAGTTCGATTTA
TTCAACAAAGCCGCCGTCCCGTCAAGTCAGCGTAATGCTCTGCCAGTGTTACAACCAATTAACCAATTCTGATTA
GAAAAACTCATCGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTATCGGTCTGCGAT
TCCGACTCGTCCAACATCAATACAACCTATTAATTTCCCCTCGTCAAAAATAAGGTTATCAAGTGAGAAATCACC
ATGAGTGACGACTGAATCCGGTGAGAATGGCAAAAGCTTATGCATTTCTTTCCAGACTTGTTCAACAGGCCAGCC
ATTACGCTCGTCATCAAAATCACTCGCATCAACCAAACCGTTATTCATTCGTGATTGCGCCTGAGCGAGACGAAA
TACGCGATCGCTGTTAAAAGGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCGGGGATCGCAGTGGTGAG
TAACCATGCATCATCAGGAGTACGGATAAAATGCTTGATGGTCGGAAGAGGCATAAATTCCGTCAGCCAGTTTAG
TCTGACCATCTCATCTGTAACATCATTGGCAACGCTACCTTTGCCATGTTTCAGAAACAACTCTGGCGCATCGGG
CTTCCCATACAATCGATAGATTGTCGCACCTGATTGCCCGACATTATCGCGAGCCCATTTATACCCATATAAATC
AGCATCCATGTTGGAATTTAATCGCGGCCTCGAGCAAGACGTTTCCCGTTGAATATGGCTCATAACACCCCTTGT
ATTACTGTTTATGTAAGCAGACAGTTTTATTGTTCATGATGATATATTTTTATCTTGTGCAATGTAACATCAGAG
ATTTTGAGACACAACGTGGCTTTCCCCCCCCCCCCTGCAGGTCGGCATCACCGGCGCCACAGGTGCGGTTGCTGG
CGCCTATATCGCCGACATCACCGATGGGGAAGATCGGGCTCGCCACTTCGGGCTCATGAGCGCTTGTTTCGGCGT
GGGTATGGTGGCAGGCCCCGTGGCCGGGGGACTGTTGGGCGCCATCTCCTTGCATGCACCATTCCTTGCGGCGGC
GGTGCTCAACGGCCTCAACCTACTACTGGGCTGCTTCCTAATGCAGGAGTCGCATAAGGGAGAGCGTCGAGTATC
TATGATTGGAAGTATGGGAATGGTGATACCCGCATTCTTCAGTGTCTTGAGGTCTCCTATCAGATTATGCCCAAC
TAAAGCAACCGGAGGAGGAGATTTCATGGTAAATTTCTCTGACTTTTGGTCATCAGTAGACTCGAACTGTGAGAC
TATCTCGGTTATGACAGCAGAAATGTCCTTCTTGGAGACAGTAAATGAAGTCCCACCAATAAAGAAATCCTTGTT
ATCAGGAACAAACTTCTTGTTTCGAACTTTTTCGGTGCCTTGAACTATAAAATGTAGAGTGGATATGTCGGGTAG
GAATGGAGCGGGCAAATGCTTACCTTCTGGACCTTCAAGAGGTATGTAGGGTTTGTAGATACTGATGCCAACTTC
AGTGACAACGTTGCTATTTCGTTCAAACCATTCCGAATCCAGAGAAATCAAAGTTGTTTGTCTACTATTGATCCA
AGCCAGTGCGGTCTTGAAACTGACAATAGTGTGCTCGTGTTTTGAGGTCATCTTTGTATGAATAAATCTAGTCTT
TGATCTAAATAATCTTGACGAGCCAAGGCGATAAATACCCAAATCTAAAACTCTTTTAAAACGTTAAAAGGACAA
GTATGTCTGCCTGTATTAAACCCCAAATCAGCTCGTAGTCTGATCCTCATCAACTTGAGGGGCACTATCTTGTTT
TAGAGAAATTTGCGGAGATGCGATATCGAGAAAAAGGTACGCTGATTTTAAACGTGAAATTTATCTCAAGATCTC
TGCCTCGCGCGTTTCGGTGATGACGGTGAAAACCTCTGACACATGCAGCTCCCGGAGACGGTCACAGCTTGTCTG
TAAGCGGATGCCGGGAGCAGACAAGCCCGTCAGGGCGCGTCAGCGGGTGTTGGCGGGTGTCGGGGCGCAGCCATG
ACCCAGTCACGTAGCGATAGCGGAGTGTATACTGGCTTAACTATGCGGCATCAGAGCAGATTGTACTGAGAGTGC
ACCATATGCGGTGTGAAATACCGCACAGATGCGTAAGGAGAAAATACCGCATCAGGCGCTCTTCCGCTTCCTCGC
TCACTGACTCGCTGCGCTCGGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCAATGCTCACGCTGTA
GGTATCTCAGTTCGGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGGTGGCCTAACTACGGCTACA
CTAGAAGGACAGTATTTGGTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGGATTTTGG
TCATGAGATTATCAAAAAGGATCTTCACCTAGATCCTTTTAAATTAAAAATGAAGTTTTAAATCAATCTAAAGTA
TATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGCCGGAAGGGCCG
AGCGCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTGCAGGCATCGTGGTGTCACGCTCGTCGTTTGGTA
TGGCTTCATTCAGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAACCAAGTCATTCTGAG
AATAGTGTATGCGGCGACCGAGTTGCTCTTGCCCGGCGTCAACACGGGATAATACCGCGCCACATAGCAGAACTT
TAAAAGTGCTCATCATTGGAAAACGTTCTTCGGGGCGAAAACTCTCAAGGATCTTACCGCTGTTGAGATCCAGTT
CGATGTAACCCACTCGTGCACCCAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGACATTAA
CCTATAAAAATAGGCGTATCACGAGGCCCTTTCGTCTTCAAGAATTAATTCTCATGTTTGACAGCTTATCATCGA
TAAGCTGACTCATGTTGGTATTGTGAAATAGACGCAGATCGGGAACACTGAAAAATAACAGTTATTATTCG
SEQ ID NO: 71
NPRENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VCAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 72
NPRENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 73
NPRENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VCAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIPPLPRHRH
SEQ ID NO: 74
NPRENFLKCFSQYIPNNATNLKLVYTQNNPLYMSVLNSTIHNLRFISDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VCAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIPPLPRHRH
SEQ ID NO: 75
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 76
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVESGATLGEVYYWVNEKNENLSLAAGYCPT
VGAGGHFSGGGYGPLMRNYGLAADNIIDAHLVNVHGKVLDRKSMGEDLFWALRGGGGESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 77
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVESGATLGEVYYWVNEKNENLSLAAGYCPT
VGAGGHFSGGGYGPLMRNYGLAADNIIDAHLVNVHGKVLDRKSMGEDLFWALRGGGGESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVKILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 78
NARENFLKCFSKHIPNNVANPKLVYTQHDQLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 79
NARENFLKCFSQHIGNRGNVYTQHDQLYMSVLNSTIHNLRFTSDTTPKPLVIVTPSNVAHIQATILCSKKVGLQI
RTRSGGHDSEGMSYISQVPFVIVDLRNMRSIKIDVHSQTAWVEAGATLGEVYYWVNEKNENLSLAAGYCPTVGAG
GHFGGGGYGPLMRNYGLAADNIIDAHLVNVHGKVLDRKSMGEDLFWALRGGGAESFGIIVAWKIRLVAVPKSTMF
SVKKIMEIHELVKLVNKWQNIAYKYDKDLLLMTHFITRNITDNQGKNKTAIHTYFSSVFLGGVDSLVDLMNKSFP
ELGIKKTDCRQLSWIDTIIFYSGVVNYDTDNENKEILLDRSAGQNGAFKIKLDYVKKPIPESVFVQILEKLYEED
IGAGMYALYPYGGIMDEISESAIPFPHRAGILYELWYICSWEKQEDNEKHLNWIRNIYNEMTPYVSKNPRLAYLN
YRDLDIGINDPKNPNNYTQARIWGEKYFGKNFDRLVKVKTLVDPNNFFRNEQSIPPLPRHRH
SEQ ID NO: 80
NARENFLKCFSQHIPNANPKLVYTQHDQLYMSVLNSTIHNLRFTSDTTPKPLVIVTPSNVAHIQATILCSKKVGL
QIRTRSGGHDSEGMSYISQVPFVIVDLRNMRSIKIDVHSQTAWVEAGATLGEVYYWVNEKNENLSLAAGYCPTVG
AGGHFGGGGYGPLMRNYGLAADNIIDAHLVNVHGKVLDRKSMGEDLFWALRGGGAESFGIIVAWKIRLVAVPKST
MFSVKKIMEIHELVKLVNKWQNIAYKYDKDLLLMTHFITRNITDNQGKNKTAIHTYFSSVFLGGVDSLVDLMNKS
FPELGIKKTDCRQLSWIDTIIFYSGVVNYDTDNFNKEILLDRSAGQNGAFKIKLDYVKKPIPESVFVQILEKLYE
EDIGAGMYALYPYGGIMDEISESAIPFPHRAGILYELWYICSWEKQEDNEKHLNWIRNIYNFMTPYVSKNPRLAY
LNYRDLDIGINDPKNPNNYTQARIWGEKYFGKNFDRLVKVKTLVDPNNFFRNEQSIPPLPRHRH
SEQ ID NO: 81
NARENLLKCLSQHGDGTGKTVYTQNNPLYMSVLNSTIHNLRFTSDTTPKPLVIVTPSNVAKIQATILCSKKVGLQ
IRTRSGGHDSEGMSYISQVPFVIVDLRNMRSIKIDVHSQTAWVEAGATLGEVYYWVNEKNENLSLAAGYCPTVGA
GGHFGGGGYGPLMRNYGLAADNIIDAHLVNVHGKVLDRKSMGEDLFWALRGGGAESFGIIVAWKIRLVAVPKSTM
FSVKKIMEIHELVKLVNKWQNIAYKYDKDLLLMTHFITRNITDNQGKNKTAIHTYFSSVFLGGVDSLVDLMNKSF
PELGIKKTDCRQLSWIDTIIFYSGVVNYDTDNENKEILLDRSAGQNGAFKIKLDYVKKPIPESVFVQILEKLYEE
DIGAGMYALYPYGGIMDEISESAIPFPHRAGILYELWYICSWEKQEDNEKHLNWIRNIYNEMTPYVSKNPRLAYL
NYRDLDIGINDPKNPNNYTQARIWGEKYFGKNFDRLVKVKTLVDPNNFFRNEQSIPPLPRHRH
SEQ ID NO: 82
NARENLLKCLSQHDGTGKTVYTQNNPLYMSVLNSTIHNLRFTSDTTPKPLVIVTPSNVAKIQATILCSKKVGLQI
RTRSGGHDSEGMSYISQVPFVIVDLRNMRSIKIDVHSQTAWVEAGATLGEVYYWVNEKNENLSLAAGYCPTVGAG
GHFGGGGYGPLMRNYGLAADNIIDAHLVNVHGKVLDRKSMGEDLFWALRGGGAESFGIIVAWKIRLVAVPKSTMF
SVKKIMEIHELVKLVNKWQNIAYKYDKDLLLMTHFITRNITDNQGKNKTAIHTYFSSVFLGGVDSLVDLMNKSFP
ELGIKKTDCRQLSWIDTIIFYSGVVNYDTDNENKEILLDRSAGQNGAFKIKLDYVKKPIPESVFVQILEKLYEED
IGAGMYALYPYGGIMDEISESAIPFPHRAGILYELWYICSWEKQEDNEKHLNWIRNIYNEMTPYVSKNPRLAYLN
YRDLDIGINDPKNPNNYTQARIWGEKYFGKNFDRLVKVKTLVDPNNFFRNEQSIPPLPRHRH
SEQ ID NO: 83
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 84
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYISSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 85
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDSRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 86
NARENFLKCFSQHIPNDATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSFPGGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 87
NARENFLKCFSQHIPNNATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNEDLSFPGGYCPT
VGAGGHFGGGGYGPLMRNYGLAADNIIDAHLVNVHGKVLDRKSMGEDLFWALRGGGAESFGIIVAWKIRLVAVPK
SSTMFSVKKIMEIHELVKLVNKWQNIAYKYDKDLLLMTHFITRNITDNQGKNKTAIHTYFSSVFLGGVDSLVDLM
NKSFPELGIKKTDCRQLSWIDTIIFYSGVVNYDTDNENKEILLDRSAGQNGAFKIKLDYVKKPIPESVFVQILEK
LYEEDIGAGMYALYPYGGIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNEQSIPPLPRHRH
SEQ ID NO: 88
NARENFLKCFSQHIPNDATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAENFGIIVAWKIRLVAVPK
STMFSVKKNMEIHALVKLVNKWQNIAYKYDKDLLLMTHFITRNITDNQGKNKTAIHVYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 89
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGGTGSGTFKKEILLDRSAGQNGAFKIKLDYVKKPIPESVFVQILEKLYE
EDIGAGMYALYPYGGIMDEISESAIPFPHRAGILYELWYICSWEKQEDNEKHLNWIRNIYNEMTPYVSKNPRLAY
LNYRDLDIGINDPKNPNNYTQARIWGEKYFGKNFDRLVKVKTLVDPNNFFRNEQSIPPLPRHRH
SEQ ID NO: 90
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGGTGSGTKKEILLDRSAGQNGAFKIKLDYVKKPIPESVFVQILEKLYEE
DIGAGMYALYPYGGIMDEISESAIPFPHRAGILYELWYICSWEKQEDNEKHLNWIRNIYNEMTPYVSKNPRLAYL
NYRDLDIGINDPKNPNNYTQARIWGEKYFGKNFDRLVKVKTLVDPNNFFRNEQSIPPLPRHRH
SEQ ID NO: 91
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGGDGTGKKEILLDRSAKGNGAFKIKLDYVKKPIPESVFVQILEKLYEED
IGAGMYALYPYGGIMDEISESAIPFPHRAGILYELWYICSWEKQEDNEKHLNWIRNIYNEMTPYVSKNPRLAYLN
YRDLDIGINDPKNPNNYTQARIWGEKYFGKNFDRLVKVKTLVDPNNFFRNEQSIPPLPRHRH
SEQ ID NO: 92
NARENFLKCFSQHIPNNATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STIFSVKKIMEIHELVKLVNKWQNIAYKYDKDLLLMTHFITKNITDNQGKNKTAIHTYFSSVFLGGVDSLVDLMN
KSFPELGIKKTDCRQFSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTKVDPNNFFRNEQSIPPLPRHRH
SEQ ID NO: 93
NARENFLKCFSQHIPNNATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGSVLDRQSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKQPIPESVFVKILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 94
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNPTPNTAIHTYFSSVFLGGVDSLVDLMNKSFPE
LGIKKTDCRQLSWIDTIIFYSGVVNYDTDNENKEILLDRSAGQNGAFKIKLDYVKKPIPESVFVQILEKLYEEDI
GAGMYALYPYGGIMDEISESAIPFPHRAGILYELWYICSWEKQEDNEKHLNWIRNIYNEMTPYVSKNPRLAYLNY
RDLDIGINDPKNPNNYTQARIWGEKYFGKNFDRLVKVKTLVDPNNFFRNEQSIPPLPRHRH
SEQ ID NO: 95
NAREDFLKCFSQHIPNNATNLKLVYTQNNPLYMSVLNSTIHNLRFTSDTTPKPLVIVTPSHVSHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRKYGLAADNIIDAHLVNVHGKVLDRKSMGEDLFWALRGGGAESFGIIVAWKIRLVAVPK
STMFSVKKNGAIHELVKLVNKWQNIAYKYDEDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKKPIPESVFVKILEKL
YEEDIGAGMYALYPYGGIMDEKSESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 96
NARENFLKCFSQHIPNNATNLKLVYTQNNPLYQSVLNSTIHNLRFTSDTTPKPLVIVTPSHVSHIQATILCSKKV
GLQIRTRSGGHDSEGLSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKNVEIHALVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGTVYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 97
NARENFLKCFSQYIPNNATNLKLVYTQNNPLYMSVLDSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 98
NARENFLKCFSQYIPNDATNLKLVYTQNNPLYMSVLDSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 99
NARENFLKCFSQYIPNDATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMQ
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 100
NARENFLKCFSQHIPNNATNLKLVYTQNNPLYMSVLDSTIHNLRFTSDTTPKPLVIVTPSHVSHIQATILCSKKV
GLQIRTRSGGHDSEGMSYISQVPFVIVDLRNMRSIKIDVHSQTAWVESGATLGEVYYWVNEKNENLSLAAGYCPT
VGAGGHFSGGGYGPLMRNYGLAADNIIDAHLVNVHGKVLDRKSMGEDLFWALRGGGGESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVKILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 101
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYICSWEKQEDNEKHLNWIRNIYNFMTPYVSKNPRL
AYLNYRDLDIGINDPKNPNDYTQARIWGEKYFGKNFDRLVKVKTLVDPNNFFRNEQSIPPLPRHRH
SEQ ID NO: 102
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAENFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 103
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGADSFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 104
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGADNFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 105
NARENFLKCFSQYIPNNATNLKLVYTQNNPLYMSVLDSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 106
NARENFLKCFSQHIPNNATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STIFSVKKIMEIHELVKLVNKWQNIAYKYDKDLLLMTHFITKNITDNQGKNKTAIHTYFSSVFLGGVDSLVDLMN
KSFPELGIKKTDCRQFSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTKVDPNNFFRNEQSIPPLPRHRH
SEQ ID NO: 107
NARENFLKCFSQHIPNDATNLKLVYTQNNPLYMSVLNSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSFPGGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 108
NARENFLKCFSQHIPNNATNLKLVYTQNNPLYMSVLDSTIHNLRFTSDTTPKPLVIVTPSNVAHIQATILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STIFSVKKIMEIHELVKLVNKWQNIAYKYDKDLLLMTHFITKNITDNQGKNKTAIHTYFSSVFLGGVDSLVDLMN
KSFPELGIKKTDCRQFSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTKVDPNNFFRNEQSIPPLPRHRH
SEQ ID NO: 109
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDARQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 110
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDTRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 111
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDPRQLSWIDTIIFYSGVVNYDTDNENKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 112
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDSRQLSWIDTIIFYSGVVNYDTDNFNKEILLDRSAGQNGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 113
NARENFLKCFSQYIPNNATNLKLVYTQNNPLYMSVLNSTIQNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNENKEILLDRSAGQKGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 114
NARENFLKCFSQYIPNNATNLKLVYTQNNPLYMSVLNSTIQNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQRGAFKIKLDYVKKPIPESVFVQILEKL
YEEDIGAGMYALYPYGGIMDEISESAIPFPHRAGILYELWYIASWEKQEDNEKHLNWIRNIYNEMTPYVSKNPRL
AYLNYRDLDIGINDPKNPNNYTQARIWGEKYFGKNFDRLVKVKTLVDPNNFFRNEQSIPPLPRHRH
SEQ ID NO: 115
NARENFLKCFSQYIPNNATNLKLVYTQNNPLYMSVLNSTIHNLRFTSDTTPKPLVIVTPSHVSHIQGTILCSKKV
GLQIRTRSGGHDSEGMSYISQVPFVIVDLRNMRSIKIDVHSQTAWVEAGATLGEVYYWVNEKNENLSLAAGYCPT
VGAGGHFGGGGYGPLMRNYGLAADNIIDAHLVNVHGKVLDRKSMGEDLFWALRGGGAESFGIIVAWKIRLVAVPK
STMFSVKKIMEIHELVKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQRGAFKIKLDYVKKPIPESVFVQILEKL
YEEDIGAGMYALYPYGGIMDEISESAIPFPHRAGILYELWYIASWEKQEDNEKHLNWIRNIYNFMTPYVSKNPRL
AYLNYRDLDIGINDPKNPNNYTQARIWGEKYFGKNFDRLVKVKTLVDPNNFFRNEQSIPPLPRHRH
SEQ ID NO: 116
MRFPSIFTAVLFAASSALAAPVNTTTEDETAQIPAEAVIGYSDLEGDFDVAVLPFSNSTNNGLLFINTTIASIAA
KEEGVSLEKREAEA
SEQ ID NO: 117
MQYKKTLVASALAATTLAAYAPSEPWSTLTPTATYSGGVTDYASTFGIAVQPISTTSSASSAATTASLEKREADA
SEQ ID NO: 118
MRFPSIFTAVLFAASSALAARAYAPSEPWSTLTPTATYSGGVTDYASTFGIAVQPISTTSSASSAATTASSASLE
KREADA
SEQ ID NO: 119
MRFPSIFTAVLFAASSALAARAYAPSEPWSTLTPTATYSGGVTDYASTFGIAVQPISTTSSASSAATTASSARAY
APSEPWSTLTPTATYSGGVTDYASTFGIAVQPISTTSSASSAATTASSASLEKREADA
SEQ ID NO: 120
MLLQAFLFLLAGFAAKISAGSASLEKREADA
SEQ ID NO: 121
MFSPILSLEIILALATLQSVFAASLEKREADA
SEQ ID NO: 122
MYRNLIIATALTCGAYSAYVPSEPWSTLTPDASLESALKDYSQTFGIAIKSLDADGSASLEKREADA
SEQ ID NO: 123
MRQVWFSWIVGLFLCFFNVSSAASLEKREADA
SEQ ID NO: 124
MRQVWFSWIVGLFLCFFNVSSAAPVNTTTEDETAQIPAEAVIGYSDLEGDEDVAVLPFSNSTNNGLLFINTTIAS
IAAKEEGASLEKREADA
SEQ ID NO: 125
MFNLKTILISTLASIAVAASLEKREADA
SEQ ID NO: 126
YPYDVPDYA
SEQ ID NO: 127
HHHHHH
SEQ ID NO: 128
HNHNHNHN
SEQ ID NO: 129
TATCCATATGATGTTCCAGATTATGCT

### Enumerated Embodiments

The following enumerated embodiments are provided, the numbering of which is not to be construed as designating levels of importance.

Embodiment 1 provides a recombinant vector comprising a promoter, secretion tag, gene of interest (GOI), and optionally a purification and/or a detection tag, wherein the gene of interest comprises a polynucleotide sequence encoding a polypeptide with at least 90%, 95%, 98%, or 99.9% sequence identity to the sequence as set forth in any one of SEQ ID NOs: 72-115.

Embodiment 2 provides the recombinant vector of embodiment 1, wherein the polynucleotide sequence encodes a polypeptide as set forth in any one of SEQ ID NOs: 72-115.

Embodiment 3 provides the recombinant vector of embodiment 1, wherein the polynucleotide sequence comprises the sequence as set forth in any one of SEQ ID NOs: 2-45, or a sequence comprising at least 90%, 95%, 98%, 99%, or 99.9% sequence identity to the sequence as set forth in any one of SEQ ID NOs: 2-45.

Embodiment 4 provides the recombinant vector of embodiment 1, wherein the gene of interest is operably linked to a promoter.

Embodiment 5 provides the recombinant vector of embodiment 4, wherein the promoter is a polynucleotide as set forth in any one of SEQ ID NOs: 59-68.

Embodiment 6 provides the recombinant vector of embodiment 5, wherein the promoter has a polynucleotide sequence comprising at least 90%, 95%, 98%, or 99% sequence identity to the sequence of SEQ ID NOs: 59-68.

Embodiment 7 provides the recombinant vector of embodiment 4, wherein the promoter is a constitutive promoter or an inducible promoter.

Embodiment 8 provides the recombinant vector of embodiment 4, wherein the promoter is selected from a group consisting of an alcohol oxidase 1 promoter (pAOX1), an alcohol oxidase 2 promoter (pAOX2), a dihydroxyacetone kinase promoter (pDAK), an S-hydroxymethyl-glutathione hydrolase promoter (pFGH), an NAD⁺-dependent formylglutathione dehydrogenase promoter (pFDH), a fructose 1,6-bisphosphate aldolase promoter (pFBA2), a peroxisomal membrane signal receptor PTS1 promoter (pPEX5), a peroxisomal protein Pex8p promoter (pPEX8), an alcohol dehydrogenase 2 promoter (pADH2) and a peroxin Pex14p promoter (pPEX14).

Embodiment 9 provides the recombinant vector of embodiment 1, comprising a secretion tag as set forth in any one of SEQ ID NOs: 46-55 and 130.

Embodiment 10 provides the recombinant vector of embodiment 9, wherein the sequence of the secretion tag is at least 90%, 95%, 98%, 99%, or 100% identical to SEQ ID NOs: 46-55 and 130.

Embodiment 11 provides the recombinant vector of embodiment 1, comprising a purification/detection tag as set forth in any one of SEQ ID NOs: 56-58 and 129.

Embodiment 12 provides the recombinant vector of embodiment 11, wherein the purification/detection tag is selected from the group consisting of a human influenza hemagglutinin (HA) tag, a His 6 tag and an HN tag.

Embodiment 13 provides a host expression system comprising the recombinant vector of embodiment 1.

Embodiment 14 provides the host expression system of embodiment 13, comprising a microbial cell, a yeast cell, a plant cell, or an animal cell.

Embodiment 15 provides the host expression system of embodiment 14, wherein the yeast cell is selected from the group consisting of Pichia pastoris, Pichia angusta, Pichia guillermordii, Pichia methanolica, Pichia inositovera, Hansenula polymorpha, Candida boidinii, and Yarrowia lipolytica.

Embodiment 16 provides a method of producing a cannabinoid compound, comprising reacting a cannabinoid precursor with an enzyme that is expressed by the host expression system of embodiment 13, wherein the cannabinoid precursor is compound of Formula I (see below): wherein R₁ is H or —COOH and R₂ is a linear or branched CH₃, C₂H₅, C₃H₇, C₄H₉, C₅H₁₀, C₆H₁₃, C₇H₁₅ or C₈H₁₇ group.

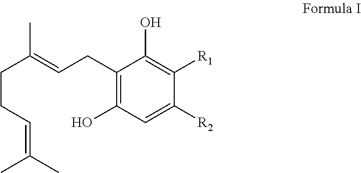

Embodiment 17 provides a polypeptide comprising the amino acid sequence of any one of SEQ ID NOs: 72-115 or an amino acid sequence comprising at least 90%, 95%, 98%, or 99.9% sequence identity to any one of SEQ ID NOs: 72-115.

Embodiment 18 provides the polypeptide of embodiment 17, wherein the polypeptide comprises the amino acid sequence of SEQ ID NO: 83, 86, 92, or 97 or an amino acid sequence comprising at least 90%, 95%, 98%, or 99.9% sequence identity to SEQ ID NO: 83, 86, 92, or 97.

Embodiment 19 provides a nucleic acid comprising a polynucleotide sequence encoding a polypeptide comprising the amino acid sequence of any one of SEQ ID NOs: 72-115 or an amino acid sequence comprising at least 90%, 95%, 98%, or 99.9% sequence identity to any one of SEQ ID NOs: 72-115.

Embodiment 20 provides the nucleic acid of embodiment 19, wherein the nucleic acid comprises the polynucleotide sequence of any one of SEQ ID NOs: 2-45 or a polynucleotide sequence comprising at least 90%, 95%, 98%, or 99.9% sequence identity to any one of SEQ ID NOs: 2-45.

Embodiment 21 provides the nucleic acid of embodiment 20, wherein the nucleic acid comprises the polynucleotide sequence of SEQ ID NO: 13, 16, 22, or 27 or a polynucleotide sequence comprising at least 90%, 95%, 98%, or 99.9% sequence identity to SEQ ID NO: 13, 16, 22, or 27.

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A recombinant vector comprising a promoter, secretion tag, gene of interest (GOI), and optionally a purification and/or a detection tag, wherein the gene of interest comprises a polynucleotide sequence encoding a polypeptide with at least 90%, 95%, 98%, or 99.9% sequence identity to the sequence as set forth in any one of SEQ ID NOs: 72-115.

2. The recombinant vector of claim 1, wherein the polynucleotide sequence encodes a polypeptide as set forth in any one of SEQ ID NOs: 72-115.

3. The recombinant vector of claim 1, wherein the polynucleotide sequence comprises the sequence as set forth in any one of SEQ ID NOs: 2-45, or a sequence comprising at least 90%, 95%, 98%, 99%, or 99.9% sequence identity to the sequence as set forth in any one of SEQ ID NOs: 2-45.

4. The recombinant vector of claim 1, wherein the gene of interest is operably linked to a promoter.

5. The recombinant vector of claim 4, wherein the promoter is a polynucleotide as set forth in any one of SEQ ID NOs: 59-68.

6. The recombinant vector of claim 5, wherein the promoter has a polynucleotide sequence comprising at least 90%, 95%, 98%, or 99% sequence identity to the sequence of SEQ ID NOs: 59-68.

7. The recombinant vector of claim 4, wherein the promoter is a constitutive promoter or an inducible promoter.

8. The recombinant vector of claim 4, wherein the promoter is selected from a group consisting of an alcohol oxidase 1 promoter (pAOX1), an alcohol oxidase 2 promoter (pAOX2), a dihydroxyacetone kinase promoter (pDAK), an S-hydroxymethyl-glutathione hydrolase promoter (pFGH), an NAD-dependent formylglutathione dehydrogenase promoter (pFDH), a fructose 1,6-bisphosphate aldolase promoter (pFBA2), a peroxisomal membrane signal receptor PTS1 promoter (pPEX5), a peroxisomal protein Pex8p promoter (pPEX8), an alcohol dehydrogenase 2 promoter (pADH2) and a peroxin Pex14p promoter (pPEX14).

9. The recombinant vector of claim 1, comprising a secretion tag as set forth in any one of SEQ ID NOs: 46-55 and 130.

10. The recombinant vector of claim 9, wherein the sequence of the secretion tag is at least 90%, 95%, 98%, 99%, or 100% identical to SEQ ID NOs: 46-55 and 130.

11. The recombinant vector of claim 1, comprising a purification/detection tag as set forth in any one of SEQ ID NOs: 56-58 and 129.

12. The recombinant vector of claim 11, wherein the purification/detection tag is selected from the group consisting of a human influenza hemagglutinin (HA) tag, a His 6 tag and an HN tag.

13. A host expression system comprising the recombinant vector of claim 1.

14. The host expression system of claim 13, comprising a microbial cell, a yeast cell, a plant cell, or an animal cell.

15. The host expression system of claim 14, wherein the yeast cell is selected from the group consisting of Pichia pastoris, Pichia angusta, Pichia guillermordii, Pichia methanolica, Pichia inositovera, Hansenula polymorpha, Candida boidinii, and Yarrowia lipolytica.

16. A method of producing a cannabinoid compound, comprising reacting a cannabinoid precursor with an enzyme that is expressed by the host expression system of claim 13, wherein the cannabinoid precursor is compound of Formula I (see below): wherein R₁ is H or —COOH and R₂ is a linear or branched CH₃, C₂H₅, C₃H₇, C₄H₉, C₅H₁₀, C₆H₁₃, C₇H₁₅ or C₈H₁₇ group.

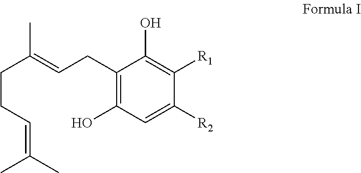

17. A polypeptide comprising the amino acid sequence of any one of SEQ ID NOs: 72-115 or an amino acid sequence comprising at least 90%, 95%, 98%, or 99.9% sequence identity to any one of SEQ ID NOs: 72-115.

18. The polypeptide of claim 17, wherein the polypeptide comprises the amino acid sequence of SEQ ID NO: 83, 86, 92, or 97 or an amino acid sequence comprising at least 90%, 95%, 98%, or 99.9% sequence identity to SEQ ID NO: 83, 86, 92, or 97.

19. A nucleic acid comprising a polynucleotide sequence encoding a polypeptide comprising the amino acid sequence of any one of SEQ ID NOs: 72-115 or an amino acid sequence comprising at least 90%, 95%, 98%, or 99.9% sequence identity to any one of SEQ ID NOs: 72-115.

20. The nucleic acid of claim 19, wherein the nucleic acid comprises the polynucleotide sequence of any one of SEQ ID NOs: 2-45 or a polynucleotide sequence comprising at least 90%, 95%, 98%, or 99.9% sequence identity to any one of SEQ ID NOs: 2-45.

21. The nucleic acid of claim 20, wherein the nucleic acid comprises the polynucleotide sequence of SEQ ID NO: 13, 16, 22, or 27 or a polynucleotide sequence comprising at least 90%, 95%, 98%, or 99.9% sequence identity to SEQ ID NO: 13, 16, 22, or 27.


SEQUENCE LISTING (130 sequences)

SEQ ID NO: 1 - DNA, 1551 bp, ARTIFICIAL SEQUENCE
  Synthetic ssequence
aacccaagagaaaacttcttgaagtgtttttcccagtacatcccaaacaacgctacaaacttgaagttggtttacactcagaacaacccattgtacatgtccgttttgaactccacaatccacaacttgagattcacttccgacactactccaaagccattggttatcgttactccatcccacgtttcccacatccagggtactattttgtgttccaagaaggttggattgcagatcagaacaagatccggtggtcacgactctgaaggtatgtcctacatttcccaggttcctttcgttatcgttgacttgagaaacatgagatccatcaagatcgacgttcactcccagactgcttgggttgaagctggtgctactttgggtgaagtttactactgggttaacgagaagaacgagaacttgtccttggctgctggttactgtccaactgtttgtgctggtggtcatttcggtggtggtggttatggtccattgatgagaaactacggtttggctgctgacaacatcatcgacgctcacttggttaacgttcacggtaaggttttggacagaaagtccatgggtgaggacttgttctgggctttgagaggtggtggtgctgaatccttcggtattatcgttgcttggaagatcagattggttgctgttccaaagtccactatgttctccgttaagaaaatcatggaaatccacgaattggttaagttggttaacaagtggcagaacattgcttacaagtacgacaaggatttgttgttgatgactcacttcatcactagaaacatcactgacaaccagggtaagaacaagactgctatccacacttacttctcttccgttttcttgggtggtgttgactccttggttgatttgatgaacaagtccttcccagagttgggtatcaagaaaactgactgtagacagttgtcctggatcgacactatcatcttctactccggtgttgttaactacgacacagacaacttcaacaaagagatcttgttggacagatccgctggacagaacggtgctttcaagatcaagttggactacgttaagaagccaatcccagagtccgttttcgttcagattttggagaagttgtacgaagaggacatcggtgctggtatgtacgctttgtacccatacggtggtatcatggacgaaatttccgagtccgctattccattcccacacagagctggtatcttgtacgagttgtggtacatctgttcttgggagaagcaagaggacaacgagaagcacttgaactggatcagaaacatctacaacttcatgactccatacgtttccaagaacccaagattggcttacttgaactacagagacttggacatcggaatcaacgacccaaagaaccctaacaactacactcaggctagaatctggggtgaaaagtacttcggtaagaacttcgacagattggttaaggttaagactttggttgacccaaacaatttcttcagaaacgagcagtccatccctccattgccaagacatagacattaa

SEQ ID NO: 2 - DNA, 1551 bp, ARTIFICIAL SEQUENCE
  Synthetic ssequence
aacccaagagaaaacttcttgaagtgtttttcccagtacatcccaaacaacgctacaaacttgaagttggtttacactcagaacaacccattgtacatgtccgttttgaactccacaatccacaacttgagattcacttccgacactactccaaagccattggttatcgttactccatcccacgtttcccacatccagggtactattttgtgttccaagaaggttggattgcagatcagaacaagatccggtggtcacgactctgaaggtatgtcctacatttcccaggttcctttcgttatcgttgacttgagaaacatgagatccatcaagatcgacgttcactcccagactgcttgggttgaagctggtgctactttgggtgaagtttactactgggttaacgagaagaacgagaacttgtccttggctgctggttactgtccaactgttggtgctggtggtcatttcggtggtggtggttatggtccattgatgagaaactacggtttggctgctgacaacatcatcgacgctcacttggttaacgttcacggtaaggttttggacagaaagtccatgggtgaggacttgttctgggctttgagaggtggtggtgctgaatccttcggtattatcgttgcttggaagatcagattggttgctgttccaaagtccactatgttctccgttaagaaaatcatggaaatccacgaattggttaagttggttaacaagtggcagaacattgcttacaagtacgacaaggatttgttgttgatgactcacttcatcactagaaacatcactgacaaccagggtaagaacaagactgctatccacacttacttctcttccgttttcttgggtggtgttgactccttggttgatttgatgaacaagtccttcccagagttgggtatcaagaaaactgactgtagacagttgtcctggatcgacactatcatcttctactccggtgttgttaactacgacacagacaacttcaacaaagagatcttgttggacagatccgctggacagaacggtgctttcaagatcaagttggactacgttaagaagccaatcccagagtccgttttcgttcagattttggagaagttgtacgaagaggacatcggtgctggtatgtacgctttgtacccatacggtggtatcatggacgaaatttccgagtccgctattccattcccacacagagctggtatcttgtacgagttgtggtacatctgttcttgggagaagcaagaggacaacgagaagcacttgaactggatcagaaacatctacaacttcatgactccatacgtttccaagaacccaagattggcttacttgaactacagagacttggacatcggaatcaacgacccaaagaaccctaacaactacactcaggctagaatctggggtgaaaagtacttcggtaagaacttcgacagattggttaaggttaagactttggttgacccaaacaatttcttcagaaacgagcagtccatccctccattgccaagacatagacattaa

SEQ ID NO: 3 - DNA, 1551 bp, ARTIFICIAL SEQUENCE
  Synthetic ssequence
aacccaagagaaaacttcttgaagtgtttttcccagtacatcccaaacaacgctacaaacttgaagttggtttacactcagaacaacccattgtacatgtccgttttgaactccacaatccacaacttgagattcacttccgacactactccaaagccattggttatcgttactccatcccacgtttcccacatccagggtactattttgtgttccaagaaggttggattgcagatcagaacaagatccggtggtcacgactctgaaggtatgtcctacatttcccaggttcctttcgttatcgttgacttgagaaacatgagatccatcaagatcgacgttcactcccagactgcttgggttgaagctggtgctactttgggtgaagtttactactgggttaacgagaagaacgagaacttgtccttggctgctggttactgtccaactgtttgtgctggtggtcatttcggtggtggtggttatggtccattgatgagaaactacggtttggctgctgacaacatcatcgacgctcacttggttaacgttcacggtaaggttttggacagaaagtccatgggtgaggacttgttctgggctttgagaggtggtggtgctgaatccttcggtattatcgttgcttggaagatcagattggttgctgttccaaagtccactatgttctccgttaagaaaatcatggaaatccacgaattggttaagttggttaacaagtggcagaacattgcttacaagtacgacaaggatttgttgttgatgactcacttcatcactagaaacatcactgacaaccagggtaagaacaagactgctatccacacttacttctcttccgttttcttgggtggtgttgactccttggttgatttgatgaacaagtccttcccagagttgggtatcaagaaaactgactgtagacagttgtcctggatcgacactatcatcttctactccggtgttgttaactacgacacagacaacttcaacaaagagatcttgttggacagatccgctggacagaacggtgctttcaagatcaagttggactacgttaagaagccaatcccagagtccgttttcgttcagattttggagaagttgtacgaagaggacatcggtgctggtatgtacgctttgtacccatacggtggtatcatggacgaaatttccgagtccgctattccattcccacacagagctggtatcttgtacgagttgtggtacatctgttcttgggagaagcaagaggacaacgagaagcacttgaactggatcagaaacatctacaacttcatgactccatacgtttcccagaacccaagattggcttacttgaactacagagacttggacatcggaatcaacgacccaaagaaccctaacaactacactcaggctagaatctggggtgaaaagtacttcggtaagaacttcgacagattggttaaggttaagactttggttgacccaaacaatttcttcagaaacgagcagtccatccctccattgccaagacatagacattaa

SEQ ID NO: 4 - DNA, 1551 bp, ARTIFICIAL SEQUENCE
  Synthetic ssequence
aacccaagagaaaacttcttgaagtgtttttcccagtacatcccaaacaacgctacaaacttgaagttggtttacactcagaacaacccattgtacatgtccgttttgaactccacaatccacaacttgagattcatttccgacactactccaaagccattggttatcgttactccatcccacgtttcccacatccagggtactattttgtgttccaagaaggttggattgcagatcagaacaagatccggtggtcacgactctgaaggtatgtcctacatttcccaggttcctttcgttatcgttgacttgagaaacatgagatccatcaagatcgacgttcactcccagactgcttgggttgaagctggtgctactttgggtgaagtttactactgggttaacgagaagaacgagaacttgtccttggctgctggttactgtccaactgtttgtgctggtggtcatttcggtggtggtggttatggtccattgatgagaaactacggtttggctgctgacaacatcatcgacgctcacttggttaacgttcacggtaaggttttggacagaaagtccatgggtgaggacttgttctgggctttgagaggtggtggtgctgaatccttcggtattatcgttgcttggaagatcagattggttgctgttccaaagtccactatgttctccgttaagaaaatcatggaaatccacgaattggttaagttggttaacaagtggcagaacattgcttacaagtacgacaaggatttgttgttgatgactcacttcatcactagaaacatcactgacaaccagggtaagaacaagactgctatccacacttacttctcttccgttttcttgggtggtgttgactccttggttgatttgatgaacaagtccttcccagagttgggtatcaagaaaactgactgtagacagttgtcctggatcgacactatcatcttctactccggtgttgttaactacgacacagacaacttcaacaaagagatcttgttggacagatccgctggacagaacggtgctttcaagatcaagttggactacgttaagaagccaatcccagagtccgttttcgttcagattttggagaagttgtacgaagaggacatcggtgctggtatgtacgctttgtacccatacggtggtatcatggacgaaatttccgagtccgctattccattcccacacagagctggtatcttgtacgagttgtggtacatctgttcttgggagaagcaagaggacaacgagaagcacttgaactggatcagaaacatctacaacttcatgactccatacgtttcccagaacccaagattggcttacttgaactacagagacttggacatcggaatcaacgacccaaagaaccctaacaactacactcaggctagaatctggggtgaaaagtacttcggtaagaacttcgacagattggttaaggttaagactttggttgacccaaacaatttcttcagaaacgagcagtccatccctccattgccaagacatagacattaa

SEQ ID NO: 5 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 6 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaaagcggtgctacattgggtgaagtttattactgggttaacgagaagaacgagaacctctccttggccgctggttactgtccaaccgttggtgctggtggtcatttctctggaggtggttatggtccattgatgagaaactacggacttgctgctgacaacatcatcgacgctcacttggttaacgttcacggtaaggttttggacagaaagtccatgggagaggacttgttctgggctttgcgtggtggtggtggtgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 7 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaaagcggtgctacattgggtgaagtttattactgggttaacgagaagaacgagaacctctccttggccgctggttactgtccaaccgttggtgctggtggtcatttctctggaggtggttatggtccattgatgagaaactacggacttgctgctgacaacatcatcgacgctcacttggttaacgttcacggtaaggttttggacagaaagtccatgggagaggacttgttctgggctttgcgtggtggtggtggtgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttaagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 8 - DNA, 1551 bp, ARTIFICIAL SEQUENCE
  Synthetic ssequence
aacgcaagagaaaacttcttgaagtgtttttccaagcacatccccaacaacgttgctaaccctaagttggtatacactcagcacgaccagttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 9 - DNA, 1539 bp, ARTIFICIAL SEQUENCE
  Synthetic ssequence
aacgcaagagaaaacttcttgaagtgtttttcccagcacatcggcaacaggggtaatgtatacactcagcacgaccagttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 10 - DNA, 1545 bp, ARTIFICIAL SEQUENCE
  Synthetic ssequence
aacgcaagagaaaacttcttgaagtgtttttcccagcacatccccaacgctaaccctaagttggtatacactcagcacgaccagttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 11 - DNA, 1542 bp, ARTIFICIAL SEQUENCE
  Synthetic ssequence
aacgcaagagaaaacttgttgaagtgtttgtcccagcacggtgacggcacaggaaagacagtatacactcagaataatcccttgtacatgtccgttttgaactccacaatccacaacttgagattcacatccgacactactccaaagcccttggttatcgttactccctccaacgttgccaagatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 12 - DNA, 1539 bp, ARTIFICIAL SEQUENCE
  Synthetic ssequence
aacgcaagagaaaacttgttgaagtgtttgtcccagcacgacggcacaggaaagacagtatacactcagaataatcccttgtacatgtccgttttgaactccacaatccacaacttgagattcacatccgacactactccaaagcccttggttatcgttactccctccaacgttgccaagatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 13 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 14 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 15 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactctagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 16 - DNA, 1551 bp, ARTIFICIAL SEQUENCE
  Synthetic ssequence
aacgcaagagaaaacttcttgaagtgtttttcccagcacatccccaacgatgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctcctttcctggtggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 17 - DNA, 1554 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgaggacctctcctttcctggtggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtcctcaactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 18 - DNA, 1551 bp, ARTIFICIAL SEQUENCE
  Synthetic ssequence
aacgcaagagaaaacttcttgaagtgtttttcccagcacatccccaacgatgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaaaatttcggtattatcgttgcttggaagatcagacttgttgctgttcccaagtccactatgttctccgttaagaaaaatatggaaatccacgctttggttaagttggttaacaagtggcagaacattgcttacaagtacgacaaggacttgttgttgatgactcacttcatcactagaaacatcactgacaaccagggtaagaacaaaactgctatccacgtttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 19 - DNA, 1545 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggaggcacaggaagtggaaccttcaagaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 20 - DNA, 1542 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggaggcacaggaagtggaaccaagaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 21 - DNA, 1539 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtggcgatggaacaggtaagaaagagatcttgttggacagatccgctaagggcaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 22 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatcttctccgttaagaaaatcatggaaatccacgaattggttaagttggttaacaagtggcagaacattgcttacaagtacgacaaggacttgttgttgatgactcacttcatcactaagaacatcactgacaaccagggtaagaacaaaactgctatccacacttacttctcttccgttttcttgggtggtgttgactccttggttgatttgatgaacaagtccttcccagagttgggcatcaagaaaactgactgtagacagttctcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactaaggttgaccctaacaatttcttcaggaacgagcagtccatccctccattgccaagacatagacattaa

SEQ ID NO: 23 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggttctgttttggacagacagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagcaaccaatcccagagtccgttttcgttaagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 24 - DNA, 1536 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaaccctacacccaatactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 25 - DNA, 1551 bp, ARTIFICIAL SEQUENCE
  Synthetic ssequence
aacgcaagagaagacttcttgaagtgtttttcccagcacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaagtacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaaacggagctatccacgaattggttaagttggttaacaagtggcagaacattgcttacaagtacgacgaagacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttaagattttggagaagttgtacgaagaggacatcggtgctggaatgtacgctttgtacccttacggtggcatcatggacgaaaagtccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 26 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtaccaatccgttttgaactccacaatccacaacttgagattcacatccgacactactccaaagcccttggttatcgttactccctcccacgtttcccacatccaggctactattttgtgttccaagaaggttggattgcagatcagaacaagatccggtggtcacgactctgaaggtttgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaaatgtcgaaatccacgctttggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtacagtttacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 27 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttggactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 28 - DNA, 1551 bp, ARTIFICIAL SEQUENCE
  Synthetic ssequence
aacgcaagagaaaacttcttgaagtgtttttcccagtacatccccaacgacgctacaaacttgaagttggtatacactcagaataatcccttgtacatgtccgttttggactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 29 - DNA, 1551 bp, ARTIFICIAL SEQUENCE
  Synthetic ssequence
aacgcaagagaaaacttcttgaagtgtttttcccagtacatccccaacgacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgcaaaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 30 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttggactccacaatccacaacttgagattcacatccgacactactccaaagcccttggttatcgttactccctcccacgtttcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaaagcggtgctacattgggtgaagtttattactgggttaacgagaagaacgagaacctctccttggccgctggttactgtccaaccgttggtgctggtggtcatttctctggaggtggttatggtccattgatgagaaactacggacttgctgctgacaacatcatcgacgctcacttggttaacgttcacggtaaggttttggacagaaagtccatgggagaggacttgttctgggctttgcgtggtggtggtggtgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttaagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 31 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatctgttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacgactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 32 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaaaacttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 33 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgattccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 34 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgataacttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 35 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttggactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 36 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatcttctccgttaagaaaatcatggaaatccacgaattggttaagttggttaacaagtggcagaacattgcttacaagtacgacaaggacttgttgttgatgactcacttcatcactaagaacatcactgacaaccagggtaagaacaaaactgctatccacacttacttctcttccgttttcttgggtggtgttgactccttggttgatttgatgaacaagtccttcccagagttgggcatcaagaaaactgactgtagacagttctcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactaaggttgaccctaacaatttcttcaggaacgagcagtccatccctccattgccaagacatagacattaa

SEQ ID NO: 37 - DNA, 1551 bp, ARTIFICIAL SEQUENCE
  Synthetic ssequence
aacgcaagagaaaacttcttgaagtgtttttcccagcacatccccaacgatgctacaaacttgaagttggtatacactcagaataatcccttgtacatgtccgttttgaactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctcctttcctggtggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 38 - DNA, 1551 bp, ARTIFICIAL SEQUENCE
  Synthetic ssequence
aacgcaagagaaaacttcttgaagtgtttttcccagcacatccccaacaacgctacaaacttgaagttggtatacactcagaataatcccttgtacatgtccgttttggactccacaatccacaacttgagattcacatccgacactactccaaagcccttggttatcgttactccctccaacgttgcccacatccaggctactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatcttctccgttaagaaaatcatggaaatccacgaattggttaagttggttaacaagtggcagaacattgcttacaagtacgacaaggacttgttgttgatgactcacttcatcactaagaacatcactgacaaccagggtaagaacaaaactgctatccacacttacttctcttccgttttcttgggtggtgttgactccttggttgatttgatgaacaagtccttcccagagttgggcatcaagaaaactgactgtagacagttctcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactaaggttgaccctaacaatttcttcaggaacgagcagtccatccctccattgccaagacatagacattaa

SEQ ID NO: 39 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgacgctagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 40 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgacactagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 41 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgaccctagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 42 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactctagacagttgtcctggatcgacactatcatcttctactccggtgttgtaaactacgacacagacaacttcaacaaagagatcttgttggacagatccgctggccagaacggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 43 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccagaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagaaaggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 44 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccagaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagagaggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 45 - DNA, 1551 bp, ARTIFICIAL SEQUENCE
  Synthetic ssequence
aacgcaagagaaaacttcttgaagtgtttttcccagtacatccccaacaacgctacaaacttgaagttggtatacactcagaataatcccttgtacatgtccgttttgaactccacaatccacaacttgagattcacatccgacactactccaaagcccttggttatcgttactccctcccacgtttcccacatccagggtactattttgtgttccaagaaggttggattgcagatcagaacaagatccggtggtcacgactctgaaggtatgtcctacatttcccaggttcccttcgttatcgttgaccttagaaacatgcgttccatcaagatcgacgttcactcccagactgcttgggttgaagcaggtgctacgttgggtgaagtttattactgggttaacgagaagaacgagaacctctccttggccgctggttactgtccaaccgttggtgctggtggtcatttcggtggaggtggttatggtccattgatgagaaactacggacttgctgctgacaacatcatcgacgctcacttggttaacgttcacggtaaggttttggacagaaagtccatgggagaggacttgttctgggctttgcgtggtggtggtgctgaatccttcggtattatcgttgcttggaagatcagacttgttgctgttcccaagtccactatgttctccgttaagaaaatcatggaaatccacgaattggttaagttggttaacaagtggcagaacattgcttacaagtacgacaaggacttgttgttgatgactcacttcatcactagaaacatcactgacaaccagggtaagaacaaaactgctatccacacttacttctcttccgttttcttgggtggtgttgactccttggttgatttgatgaacaagtccttcccagagttgggcatcaagaaaactgactgtagacagttgtcctggatcgacactatcatcttctactccggtgttgtaaactacgacacagacaacttcaacaaagagatcttgttggacagatccgctggccagagaggtgctttcaagatcaagttggactacgttaagaagccaatcccagagtccgttttcgttcagattttggagaagttgtacgaagaggacatcggtgctggaatgtacgctttgtacccttacggtggcatcatggacgaaatctccgagtccgctattcccttcccacaccgtgctggtatcttgtacgagttgtggtacatcgcttcttgggagaagcaagaggacaacgagaagcacttgaactggatcagaaacatctacaacttcatgactccatacgtttccaaaaaccctagattggcatacctaaactacagagacttggacatcggcatcaacgaccccaagaaccctaacaactacactcaggctagaatctggggtgaaaagtacttcggtaagaacttcgacagattggttaaggttaagactttggttgaccctaacaatttcttcaggaacgagcagtccatccctccattgccaagacatagacattaa

SEQ ID NO: 46 - DNA, 267 bp, ARTIFICIAL SEQUENCE
  Synthetic ssequence
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 47 - DNA, 225 bp, ARTIFICIAL SEQUENCE
  Synthetic ssequence
atgcaatacaaaaagactttggttgcctctgctttggccgctactacattggccgcctatgctccatctgagccttggtccactttgactccaacagccacttacagcggtggtgttaccgactacgcttccaccttcggtattgccgttcaaccaatctccactacatccagcgcatcatctgcagccaccacagctagcttggagaagagggaggctgatgct

SEQ ID NO: 48 - DNA, 243 bp, ARTIFICIAL SEQUENCE
  Synthetic ssequence
atgagattcccatccatcttcactgctgttttgttcgctgcttcttccgctttggctgctagagcctatgctccatctgagccttggtccactttgactccaacagccacttacagcggtggtgttaccgactacgcttccaccttcggtattgccgttcaaccaatctccactacatccagcgcatcatctgcagccaccacagcctcatctgctagcttggagaagagggaggctgatgct

SEQ ID NO: 49 - DNA, 498 bp, ARTIFICIAL SEQUENCE
  Synthetic ssequence
atgagattcccatccatcttcactgctgttttgttcgctgcttcttccgctttggctgctccagttaacactactactgaggacgagactgctcagattccagctgaagctgttattggttactccgacttggaaggtgacttcgacgttgctgttttgccattctccaactccactaacaacggtttgttgttcatcaacactacaatcgcttccattgctgctaaagaagagggagtttccttggagaagagagaggctgaagctcgctgcttcttccgctttggctgctccagttaacactactactgaggacgagactgctcagattccagctgaagctgttattggttactccgacttggaaggtgacttcgacgttgctgttttgccattctccaactccactaacaacggtttgttgttcatcaacactacaatcgcttccattgctgctaaagaagagggagtttccttggagaagagagaggctgaagc

SEQ ID NO: 50 - DNA, 93 bp, ARTIFICIAL SEQUENCE
  Synthetic ssequence
atgcttttgcaagctttccttttccttttggctggttttgcagccaaaatatctgcaggatccgctagcttggagaagagggaggctgaagct

SEQ ID NO: 51 - DNA, 96 bp, ARTIFICIAL SEQUENCE
  Synthetic ssequence
atgttttctcctattctaagtctggaaattattctcgctttggctactctccaatcagtctttgcggctagcttggagaagagggaggctgaagct

SEQ ID NO: 52 - DNA, 200 bp, ARTIFICIAL SEQUENCE
  Synthetic ssequence
tgtacaggaacttaataattgctactgcccttacttgcggtgcatacagtgcctacgtgccttccgaaccatggagcacactgacacctgatgctagccttgaaagtgccctcaaagattactcacaaacttttggaatagctattaagtccttagatgccgacggatccgctagcttggagaagagggaggctgaagct

SEQ ID NO: 53 - DNA, 96 bp, ARTIFICIAL SEQUENCE
  Synthetic ssequence
atgaggcaggtttggttctcttggattgtgggattgttcctatgttttttcaacgtgtcttctgctgctagcttggagaagagggaggctgatgct

SEQ ID NO: 54 - DNA, 276 bp, ARTIFICIAL SEQUENCE
  Synthetic ssequence
atgaggcaggtttggttctcttggattgtgggattgttcctatgttttttcaacgtgtcttctgctgctccagttaacactactactgaggacgagactgctcagattccagctgaagctgttattggttactccgacttggaaggtgacttcgacgttgctgttttgccattctccaactccactaacaacggtttgttgttcatcaacactacaatcgcttccattgctgctaaagaagagggagctagcttggagaagagggaggctgatgct

SEQ ID NO: 55 - DNA, 84 bp, ARTIFICIAL SEQUENCE
  Synthetic ssequence
atgttcaacctgaaaactattctcatctcaacacttgcatcgatcgctgttgccgctagcttggagaagagggaggctgatgct

SEQ ID NO: 56 - DNA, 27 bp, ARTIFICIAL SEQUENCE
  Synthetic ssequence
tacccatacgatgttccagattacgct

SEQ ID NO: 57 - DNA, 18 bp, ARTIFICIAL SEQUENCE
  Synthetic ssequence
catcaccatcaccatcac

SEQ ID NO: 58 - DNA, 24 bp, ARTIFICIAL SEQUENCE
  Synthetic ssequence
cataaccataatcacaatcacaac

SEQ ID NO: 59 - DNA, 939 bp, ARTIFICIAL SEQUENCE
  Synthetic ssequence
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 60 - DNA, 1542 bp, ARTIFICIAL SEQUENCE
  Synthetic ssequence
ttattttttgaccgaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaa

SEQ ID NO: 61 - DNA, 358 bp, ARTIFICIAL SEQUENCE
  Synthetic ssequence
tgagataacagagttgggtaactagagagaataatagacgtatgcatgattactacacaacggatgtcgcactctttccttagttaaaactatcatccaatcacaagatgcgggctggaaagacttgctcccgaaggataatcttctgcttctatctcccttcctcatatggtttcgcagggctcatgccccttcttccttcgaactgcccgatgaggaagtccttagcctatcaaagaattcgggaccatcatcgatttttagagccttacctgatcgcaatcaggatttcactactcatataaatacatcgctcaaagctccaactttgcttgttcatacaattcttgatattcac

SEQ ID NO: 62 - DNA, 1032 bp, ARTIFICIAL SEQUENCE
  Synthetic ssequence
attctggcccttgcacctgatcgcgaaggtggaaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 63 - DNA, 560 bp, ARTIFICIAL SEQUENCE
  Synthetic ssequence
atcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttcta

SEQ ID NO: 64 - DNA, 609 bp, ARTIFICIAL SEQUENCE
  Synthetic ssequence
tgtcatctgctgatgctgtgagggagaaagaagtaggggtgatacatggtttataggcaaagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaa

SEQ ID NO: 65 - DNA, 704 bp, ARTIFICIAL SEQUENCE
  Synthetic ssequence
aaattaatccataagataaggcaaatgtgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaat

SEQ ID NO: 66 - DNA, 501 bp, ARTIFICIAL SEQUENCE
  Synthetic ssequence
tccaaaccaaacggtctagcaaaaacgataactttaaagaacttttcaattggttttgtacactaccaccggtttactacctctgccttcggttcttctcctcacatttttcgcaactgggatagcgtagcctaaagtgtcacatgctcgctgctcacattccctacacaacagagattgtcagcagaggaaattgagctccaccattcaacacttgtggatttatgatagtctgtgctatcagctctcttttttttgttgctgtagaatttaccgtgctagcaaccttttaaactttgtttagctctccttccctcttccattcatctgtttcggtccgatccgtctctggtcatctcctccgcattttttttttaccgttagcgataggggtcagatcaattcaatcagttttggcaagggtatttaaaggtggcgaaatccccctccgtttgttgaacacatccaactattctcaacccaaccatctaactaatcgta

SEQ ID NO: 67 - DNA, 1212 bp, ARTIFICIAL SEQUENCE
  Synthetic ssequence
cgcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 68 - DNA, 258 bp, ARTIFICIAL SEQUENCE
  Synthetic ssequence
tgtgtctgtaaatttaaatccaaagaaaaataaagggcactacatagaacaacacaatcaacctacatagctctctttttttttttttttttttttttcttttttgttttttctcaattttcttcattttttgcattgttactactctcccattaagggaaattctaccaggaaaaccgaagaagacaaatgcaaaagggaaatatgaatacgcatgtatgcgcgcaaaaccgcacttacagagggcattaggacatt

SEQ ID NO: 69 - DNA, 9016 bp, ARTIFICIAL SEQUENCE
  Synthetic ssequence
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacggaattcgctggatcctacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 70 - DNA, 6521 bp, ARTIFICIAL SEQUENCE
  Synthetic ssequence
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatctgaattcatagctagcttggagaagaggccttaattaactgggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctaggtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccagctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 71 - protein, 516 aa, ARTIFICIAL SEQUENCE
  Synthetic ssequence
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 72 - protein, 516 aa, ARTIFICIAL SEQUENCE
  Synthetic ssequence
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 73 - protein, 516 aa, ARTIFICIAL SEQUENCE
  Synthetic ssequence
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 74 - protein, 516 aa, ARTIFICIAL SEQUENCE
  Synthetic ssequence
NPRENFLKCFSQYIPNNATNLKLVYTQNNPLYMSVLNSTIHNLRFISDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 75 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 76 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVESGATLGEVYYWVNEKNENLSLAAGYCPTVGAGGHFSGGGYGPLMRNYGLAADNIIDAHLVNVHGKVLDRKSMGEDLFWALRGGGGESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 77 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVESGATLGEVYYWVNEKNENLSLAAGYCPTVGAGGHFSGGGYGPLMRNYGLAADNIIDAHLVNVHGKVLDRKSMGEDLFWALRGGGGESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVKILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 78 - protein, 516 aa, ARTIFICIAL SEQUENCE
  Synthetic ssequence
NARENFLKCFSKHIPNNVANPKLVYTQHDQLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 79 - protein, 512 aa, ARTIFICIAL SEQUENCE
  Synthetic ssequence
NARENFLKCFSQHIGNRGNVYTQHDQLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 80 - protein, 514 aa, ARTIFICIAL SEQUENCE
  Synthetic ssequence
NARENFLKCFSQHIPNANPKLVYTQHDQLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 81 - protein, 513 aa, ARTIFICIAL SEQUENCE
  Synthetic ssequence
NARENLLKCLSQHGDGTGKTVYTQNNPLYMSVLNSTIHNLRFTSDTTPKPLVIVTPSNVAKIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 82 - protein, 512 aa, ARTIFICIAL SEQUENCE
  Synthetic ssequence
NARENLLKCLSQHDGTGKTVYTQNNPLYMSVLNSTIHNLRFTSDTTPKPLVIVTPSNVAKIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 83 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 84 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYISSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 85 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDSRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 86 - protein, 516 aa, ARTIFICIAL SEQUENCE
  Synthetic ssequence
NARENFLKCFSQHIPNDATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSFPGGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 87 - protein, 517 aa, ARTIFICIAL SEQUENCE
  Synthetic ssequence
NARENFLKCFSQHIPNNATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNEDLSFPGGYCPTVGAGGHFGGGGYGPLMRNYGLAADNIIDAHLVNVHGKVLDRKSMGEDLFWALRGGGAESFGIIVAWKIRLVAVPKSSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 88 - protein, 516 aa, ARTIFICIAL SEQUENCE
  Synthetic ssequence
NARENFLKCFSQHIPNDATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAENFGIIVAWKIRLVAVPKSTMFSVKKNMEIHALVKLVNKWQNIAYKYDKDLLLMTHFITRNITDNQGKNKTAIHVYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 89 - protein, 514 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGGTGSGTFKKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 90 - protein, 513 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGGTGSGTKKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 91 - protein, 512 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGGDGTGKKEILLDRSAKGNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 92 - protein, 516 aa, ARTIFICIAL SEQUENCE
  Synthetic ssequence
NARENFLKCFSQHIPNNATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTIFSVKKIMEIHELVKLVNKWQNIAYKYDKDLLLMTHFITKNITDNQGKNKTAIHTYFSSVFLGGVDSLVDLMNKSFPELGIKKTDCRQFSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTKVDPNNFFRNEQSIPPLPRHRH

SEQ ID NO: 93 - protein, 516 aa, ARTIFICIAL SEQUENCE
  Synthetic ssequence
NARENFLKCFSQHIPNNATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGSVLDRQSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKQPIPESVFVKILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 94 - protein, 511 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNPTPNTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 95 - protein, 516 aa, ARTIFICIAL SEQUENCE
  Synthetic ssequence
NAREDFLKCFSQHIPNNATNLKLVYTQNNPLYMSVLNSTIHNLRFTSDTTPKPLVIVTPSHVSHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRKYGLAADNIIDAHLVNVHGKVLDRKSMGEDLFWALRGGGAESFGIIVAWKIRLVAVPKSTMFSVKKNGAIHELVKLVNKWQNIAYKYDEDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVKILEKLYEEDIGAGMYALYPYGGIMDEKSESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 96 - protein, 516 aa, ARTIFICIAL SEQUENCE
  Synthetic ssequence
NARENFLKCFSQHIPNNATNLKLVYTQNNPLYQSVLNSTIHNLRFTSDTTPKPLVIVTPSHVSHIQATILCSKKVGLQIRTRSGGHDSEGLSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKNVEIHALVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGTVYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 97 - protein, 516 aa, ARTIFICIAL SEQUENCE
  Synthetic ssequence
NARENFLKCFSQYIPNNATNLKLVYTQNNPLYMSVLDSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 98 - protein, 516 aa, ARTIFICIAL SEQUENCE
  Synthetic ssequence
NARENFLKCFSQYIPNDATNLKLVYTQNNPLYMSVLDSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 99 - protein, 516 aa, ARTIFICIAL SEQUENCE
  Synthetic ssequence
NARENFLKCFSQYIPNDATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMQKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 100 - protein, 516 aa, ARTIFICIAL SEQUENCE
  Synthetic ssequence
NARENFLKCFSQHIPNNATNLKLVYTQNNPLYMSVLDSTIHNLRFTSDTTPKPLVIVTPSHVSHIQATILCSKKVGLQIRTRSGGHDSEGMSYISQVPFVIVDLRNMRSIKIDVHSQTAWVESGATLGEVYYWVNEKNENLSLAAGYCPTVGAGGHFSGGGYGPLMRNYGLAADNIIDAHLVNVHGKVLDRKSMGEDLFWALRGGGGESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVKILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 101 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNDYTQARIWGEKYFGKNFDRLVKVKTLVDPNNFFRNEQSIPPLPRHRH

SEQ ID NO: 102 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAENFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 103 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGADSFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 104 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGADNFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 105 - protein, 516 aa, ARTIFICIAL SEQUENCE
  Synthetic ssequence
NARENFLKCFSQYIPNNATNLKLVYTQNNPLYMSVLDSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 106 - protein, 516 aa, ARTIFICIAL SEQUENCE
  Synthetic ssequence
NARENFLKCFSQHIPNNATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTIFSVKKIMEIHELVKLVNKWQNIAYKYDKDLLLMTHFITKNITDNQGKNKTAIHTYFSSVFLGGVDSLVDLMNKSFPELGIKKTDCRQFSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTKVDPNNFFRNEQSIPPLPRHRH

SEQ ID NO: 107 - protein, 516 aa, ARTIFICIAL SEQUENCE
  Synthetic ssequence
NARENFLKCFSQHIPNDATNLKLVYTQNNPLYMSVLNSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSFPGGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 108 - protein, 516 aa, ARTIFICIAL SEQUENCE
  Synthetic ssequence
NARENFLKCFSQHIPNNATNLKLVYTQNNPLYMSVLDSTIHNLRFTSDTTPKPLVIVTPSNVAHIQATILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTIFSVKKIMEIHELVKLVNKWQNIAYKYDKDLLLMTHFITKNITDNQGKNKTAIHTYFSSVFLGGVDSLVDLMNKSFPELGIKKTDCRQFSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTKVDPNNFFRNEQSIPPLPRHRH

SEQ ID NO: 109 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDARQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 110 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDTRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 111 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDPRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 112 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDSRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 113 - protein, 516 aa, ARTIFICIAL SEQUENCE
  Synthetic ssequence
NARENFLKCFSQYIPNNATNLKLVYTQNNPLYMSVLNSTIQNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQKGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 114 - protein, 516 aa, ARTIFICIAL SEQUENCE
  Synthetic ssequence
NARENFLKCFSQYIPNNATNLKLVYTQNNPLYMSVLNSTIQNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQRGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 115 - protein, 516 aa, ARTIFICIAL SEQUENCE
  Synthetic ssequence
NARENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQRGAFKIKLDYVKKPIPESVFVQILEKLYEEDIGAGMYALYPYGGIMDEISESAIPFPHRAGILYELWYIASWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 116 - protein, 89 aa, ARTIFICIAL SEQUENCE
  Synthetic ssequence
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 117 - protein, 75 aa, ARTIFICIAL SEQUENCE
  Synthetic ssequence
MQYKKTLVASALAATTLAAYAPSEPWSTLTPTATYSGGVTDYASTFGIAVQPISTTSSASSAATTASLEKREADA

SEQ ID NO: 118 - protein, 81 aa, ARTIFICIAL SEQUENCE
  Synthetic ssequence
MRFPSIFTAVLFAASSALAARAYAPSEPWSTLTPTATYSGGVTDYASTFGIAVQPISTTSSASSAATTASSASLEKREADA

SEQ ID NO: 119 - protein, 133 aa, ARTIFICIAL SEQUENCE
  Synthetic ssequence
MRFPSIFTAVLFAASSALAARAYAPSEPWSTLTPTATYSGGVTDYASTFGIAVQPISTTSSASSAATTASSARAYAPSEPWSTLTPTATYSGGVTDYASTFGIAVQPISTTSSASSAATTASSASLEKREADA

SEQ ID NO: 120 - protein, 31 aa, ARTIFICIAL SEQUENCE
  Synthetic ssequence
MLLQAFLFLLAGFAAKISAGSASLEKREADA

SEQ ID NO: 121 - protein, 32 aa, ARTIFICIAL SEQUENCE
  Synthetic ssequence
MFSPILSLEIILALATLQSVFAASLEKREADA

SEQ ID NO: 122 - protein, 67 aa, ARTIFICIAL SEQUENCE
  Synthetic ssequence
MYRNLIIATALTCGAYSAYVPSEPWSTLTPDASLESALKDYSQTFGIAIKSLDADGSASLEKREADA

SEQ ID NO: 123 - protein, 32 aa, ARTIFICIAL SEQUENCE
  Synthetic ssequence
MRQVWFSWIVGLFLCFFNVSSAASLEKREADA

SEQ ID NO: 124 - protein, 92 aa, ARTIFICIAL SEQUENCE
  Synthetic ssequence
MRQVWFSWIVGLFLCFFNVSSAAPVNTTTEDETAQIPAEAVIGYSDLEGDFDVAVLPFSNSTNNGLLFINTTIASIAAKEEGASLEKREADA

SEQ ID NO: 125 - protein, 28 aa, ARTIFICIAL SEQUENCE
  Synthetic ssequence
MFNLKTILISTLASIAVAASLEKREADA

SEQ ID NO: 126 - protein, 9 aa, ARTIFICIAL SEQUENCE
  Synthetic ssequence
YPYDVPDYA

SEQ ID NO: 127 - protein, 6 aa, ARTIFICIAL SEQUENCE
  Synthetic ssequence
HHHHHH

SEQ ID NO: 128 - protein, 8 aa, ARTIFICIAL SEQUENCE
  Synthetic ssequence
HNHNHNHN

SEQ ID NO: 129 - DNA, 27 bp, ARTIFICIAL SEQUENCE
  Synthetic ssequence
tatccatatgatgttccagattatgct

SEQ ID NO: 130 - DNA, 267 bp, ARTIFICIAL SEQUENCE
  Synthetic ssequence
atgagattcccatccatcttcactgctgttttgttcgctgcttcttccgctttggctgctccagttaacactactactgaggacgagactgctcagattccagctgaagctgttattggttactccgacttggaaggtgacttcgacgttgctgttttgccattctccaactccactaacaacggtttgttgttcatcaacactacaatcgcttccattgctgctaaagaagagggagtttccttggagaagagagaggctgaagct